SEQUENCE LISTING (82 sequences)

SEQ ID NO: 1 - DNA, 20996 bp, synthetic construct
  misc_feature 1..20996: note = Plasmid
  source 1..20996: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattaggtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 2 - DNA, 20027 bp, synthetic construct
  misc_feature 1..20027: note = Plasmid
  source 1..20027: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattgagtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 3 - DNA, 19659 bp, synthetic construct
  misc_feature 1..19659: note = Plasmid
  source 1..19659: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattgagtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccatggaacgtggagctcccttctctcactatcagctaccaaaatccatctctgaattgaacttggaccagcacagcaacaacccaaccccaatgaccagctcagtcgtagtagccggcgccggtgacaagaacaatggtatcgtggtccagcagcaaccaccatgtgtggctcgtgagcaagaccaatacatgccaatcgcaaacgtcataagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggtttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtgatggccacggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 4 - DNA, 1740 bp, Brassica napus
  source 1..1740: mol_type = other DNA
atgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattag

SEQ ID NO: 5 - protein, 579 aa, Brassica napus
MNNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 6 - DNA, 1092 bp, Arabidopsis thaliana
  source 1..1092: mol_type = other DNA
atggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtga

SEQ ID NO: 7 - protein, 363 aa, Arabidopsis thaliana
MDNFLPFPSSNANSVQELSMDPNNNRSHFTTVPTYDHHQAQPHHFLPPFSYPVEQMAAVMNPQPVYLSECYPQIPVTQTGSEFGSLVGNPCLWQERGGFLDPRMTKMARINRKNAMMRSRNNSSPNSSPSELVDSKRQLMMLNLKNNVQISDKKDSYQQSTFDNKKLRVLCEKELKNSDVGSLGRIVLPKRDAEANLPKLSDKEGIVVQMRDVFSMQSWSFKYKFWSNNKSRMYVLENTGEFVKQNGAEIGDFLTIYEDESKNLYFAMNGNSGKQNEGRENESRERNHYEEAMLDYIPRDEEEASIAMLIGNLNDHYPIPNDLMDLTTDLQHHQATSSSMPPEDHAYVGSSDDQVSFNDFEWW

SEQ ID NO: 8 - DNA, 771 bp, Arabidopsis thaliana
  source 1..771: mol_type = other DNA
atgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattga

SEQ ID NO: 9 - protein, 256 aa, Arabidopsis thaliana
MSSSKHSSVFNYSALFLSLFLQQMDQNSLHHLDSPKIENEYEPDSLYDMLDKLPPLDSLLDMEDLKPNAGLHFQFHYNSFEDFFENIEVDNTIPSDIHLLTQEPYFSSDSSSSSPLAIQNDGLISNVKVEKVTVKKKRNLKKKRQDKLEMSEIKQFFDRPIMKAAKELNVGLTVLKKRCRELGIYRWPHRKLKSLNSLIKNLKNVGMEEEVKNLEEHRFLIEQEPDAELSDGTKKLRQACFKANYKRRKSLGDDYY

SEQ ID NO: 10 - DNA, 717 bp, Arabidopsis thaliana
  source 1..717: mol_type = other DNA
atggaacgtggagctcccttctctcactatcagctaccaaaatccatctctgaattgaacttggaccagcacagcaacaacccaaccccaatgaccagctcagtcgtagtagccggcgccggtgacaagaacaatggtatcgtggtccagcagcaaccaccatgtgtggctcgtgagcaagaccaatacatgccaatcgcaaacgtcataagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggtttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtga

SEQ ID NO: 11 - protein, 238 aa, Arabidopsis thaliana
MERGAPFSHYQLPKSISELNLDQHSNNPTPMTSSVVVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 12 - DNA, 1098 bp, Eutrema salsugineum
  source 1..1098: mol_type = other DNA
atggataacttcttgcccttttcctctccaaacgcaaactctgtccaagagctctctatggatctaaacaccaatcgctcacacttctcaacggtccctacttatgatcatcatcaggcgcatcatcactaccacatgttaccaccgtatccaaacattgcatgtccggtggacccaacggcggtgatgaatcctcagccgcttcactcatcggaggctttttctcggttcccgtttacgcgaaccggaagtgaattcggctctctggtttgtaaccccggtttgagacaagagagaggtggatttcttgatccacacatgacgaagatggccaggatcaacaggaagaaggcgatgataagatcaagaaacaactattgccctaattctagttcaaatgagttggttgattcaaggagacaagtggctcttaacatgaagaataattccgagactaccgctaaaaaagatctctttcgattttcctcattcgataacaagaagcttagggttttgttggtgaagcacttgaagaacagcgatgttgggtcacttgggaggatcgttctaccaaagagagaagcagaaggaaatcttccagagctatctgataaagaaggaataatggtagagatgagagatgttgactctgtgcagtcttggtctttcaaatacaagttctggtccaataacaagagcagaatgtatgtcctcgaaaacacaggagaatttgtgaagaaaaatggagtagagactggagattctttaacaatatacgaggacgaaagcaagaatctctacttctccattagaaagtactcaggcaaacaaaatgaaggaagagaagatgagtcgatggaagtcaacgacatgaacttcctcgaagatttaacgtttgattacataccaaatgacgaagaagaagattctattgcaatgctcatcggaaatctaaacgatcactattccattccaaacgatctaatggacctcacaaccgatcttgatcaagcaacctcctcgtcgccacatttggatcacctgtctgatggtcacgccacaagtgatcacgtgagctcaaacgacttcgtatggtga

SEQ ID NO: 13 - protein, 365 aa, Eutrema salsugineum
MDNFLPFSSPNANSVQELSMDLNTNRSHFSTVPTYDHHQAHHHYHMLPPYPNIACPVDPTAVMNPQPLHSSEAFSRFPFTRTGSEFGSLVCNPGLRQERGGFLDPHMTKMARINRKKAMIRSRNNYCPNSSSNELVDSRRQVALNMKNNSETTAKKDLFRFSSFDNKKLRVLLVKHLKNSDVGSLGRIVLPKREAEGNLPELSDKEGIMVEMRDVDSVQSWSFKYKFWSNNKSRMYVLENTGEFVKKNGVETGDSLTIYEDESKNLYFSIRKYSGKQNEGREDESMEVNDMNFLEDLTFDYIPNDEEEDSIAMLIGNLNDHYSIPNDLMDLTTDLDQATSSSPHLDHLSDGHATSDHVSSNDFVW

SEQ ID NO: 14 - DNA, 1389 bp, Gossypium hirsutum
  source 1..1389: mol_type = other DNA
atgaatggccaggattcatggtgtcttgtgctcaagagtattccaacatttaaaccaaaggtggcgccaactccctctcgagctctatcagtgaactcttacatgccttttctccccaccacctccacagccaccaataccatcctgaggcaaccaaacatgggttgttctcagaattcagagctttcaatctatgaaccaatgaattccaaaaacttcagttccaaacccataacaacagcaccaaactctcaattccatcatccaacacagtttatggcaccttaccaaagcctgcgactcccatacagacatcccatagagcagactgatcattctttagcgtatcctttctttctggggcagaatggaattgaatttggttcgattatcgctagtaatggggtgcaacacggatcatgtttcatgcaaacagacggggtttcgaaggcagaagagagaaggatgctggacccttacggatcaaaggctgcaaggattaagagaaagctggcccgccaaagaagccttagtttgcaaagaaatgcttcttcaggggctacagctctggtggataaaaggagactgaacagctctggtgctgacaatgatgtgaatatcaacaacgataccaaaagggatctatacatgttctgcacgccagataacaagagactgagagtgttgctgaggaaagagctaaagaacagtgatgtcgggtcccttgggagaattgtccttccaaagagagaggcggagctaaatcttccaaccctttgtgataaagaaggcatccaagtagtgctaaaagacgtatactctcaccaatcatggcccctcaaatacaagttctggtctaataataagagtagaatgtatgtgcttgaaaacacagcggattttgtgaagcaaaatggactgggaattggagatttccttaccctttacgaagatgaaggcaaaaatctccagtatttctccgtaataaaggtggaaagggttgctgctgcagagccttcaaacaatcaaccctatgattctcaaaacaactatatggacatgccatttacaggcccacttagagatgaggaagaaacatgtttagagttacttatagaacaacttaaacacacggaacatgaagaatctaacgaccttatgactttggccatggatgctacttattcgcatggcccgcctgctgccgatgaatcccaacattttgtatccgatgccaacgtagaactaactgcaaccgaactgcaaacctcgtcgacatcgttgcgtggtaaagcgagaagtgtagatgaaattcagatcaactttgatgactgctatagcggtcttgacatgctacctgatgtcaacctttacaattttttactttga

SEQ ID NO: 15 - protein, 462 aa, Gossypium hirsutum
MNGQDSWCLVLKSIPTFKPKVAPTPSRALSVNSYMPFLPTTSTATNTILRQPNMGCSQNSELSIYEPMNSKNFSSKPITTAPNSQFHHPTQFMAPYQSLRLPYRHPIEQTDHSLAYPFFLGQNGIEFGSIIASNGVQHGSCFMQTDGVSKAEERRMLDPYGSKAARIKRKLARQRSLSLQRNASSGATALVDKRRLNSSGADNDVNINNDTKRDLYMFCTPDNKRLRVLLRKELKNSDVGSLGRIVLPKREAELNLPTLCDKEGIQVVLKDVYSHQSWPLKYKFWSNNKSRMYVLENTADFVKQNGLGIGDFLTLYEDEGKNLQYFSVIKVERVAAAEPSNNQPYDSQNNYMDMPFTGPLRDEEETCLELLIEQLKHTEHEESNDLMTLAMDATYSHGPPAADESQHFVSDANVELTATELQTSSTSLRGKARSVDEIQINFDDCYSGLDMLPDVNLYNFLL

SEQ ID NO: 16 - DNA, 1281 bp, Manihot esculenta
  source 1..1281: mol_type = other DNA
atggagaatcctttttcacctttatccatagatactagcaacaccacctatgatagcgaatcaaaaatggggggttctcaaaattcatcaacatcaacacccccggcctcctctatctatggaccactgagctatccatcaacatcttcatgttcatcaactcagccttaccattgtccagtagaatacagcatgctaccaccttttcaaagcttgcagtttccacaagcatatcctcaaggccacactggccagtactcaccatcaacatatcctttgtatccgtgctggtttttgcagaatgggattgaaaaatcatgctttttgcagccaaatggggtgtcaatagagcaagaaaggagagttcttgatgcatacaagaccaaggttgcaaggactaagcgaaaacttgcacgccaaaggagccttggcaaaaatgcttcttctggtgctaatttgtctcaggttgatacaaggagattatccttgaatggtggtaacaacaatgggcaagctggcctgagcaacatcaacatcaacagagatctttataaaatctgcactccagataacaagagactgagggtgttgctaagtaaagagttgaagaacagtgatgttgggtctcttggaaggattgtccttccaaagagaggagcagaggaaaatcttccaattctttctgacaaagaaggcatgcaagtagtgattagagatctaaactctcctaaagagtggagcttgaagttcaaattttggtctaacaacaagagcagaatgtatgttcttgaaaacacaggagattttgtaaagcaaaatgggctggagattggagattcccttacactttacgaggatgagtgcaagaatctctatttctccatcaagaaggtgaaaacaccgcaaatcgaggtgtcatacaaccaaaactcgacaaaccaaaactacctatacataccacatatgtaccaagctagggatgaagaagaagcatctctgcaactacttatagaacaacttaatcataaagaacagcaagaagctaacagccttgtgactttgtctatggatattgcttcttcttataggcgcaaagaggaagagagactcagtccattcaacaatttaaccagcattagtttctattcccaaccagcacctgcagctatggaagcctcatcttcaccacacaatacaatgagaattttagatgatcactacattgatgactgctatagtggccttgatgttcttcctgatgtcaataggtataatttttag

SEQ ID NO: 17 - protein, 426 aa, Manihot esculenta
MENPFSPLSIDTSNTTYDSESKMGGSQNSSTSTPPASSIYGPLSYPSTSSCSSTQPYHCPVEYSMLPPFQSLQFPQAYPQGHTGQYSPSTYPLYPCWFLQNGIEKSCFLQPNGVSIEQERRVLDAYKTKVARTKRKLARQRSLGKNASSGANLSQVDTRRLSLNGGNNNGQAGLSNININRDLYKICTPDNKRLRVLLSKELKNSDVGSLGRIVLPKRGAEENLPILSDKEGMQVVIRDLNSPKEWSLKFKFWSNNKSRMYVLENTGDFVKQNGLEIGDSLTLYEDECKNLYFSIKKVKTPQIEVSYNQNSTNQNYLYIPHMYQARDEEEASLQLLIEQLNHKEQQEANSLVTLSMDIASSYRRKEEERLSPFNNLTSISFYSQPAPAAMEASSSPHNTMRILDDHYIDDCYSGLDVLPDVNRYNF

SEQ ID NO: 18 - DNA, 1218 bp, Cephalotus follicularis
  source 1..1218: mol_type = other DNA
atggaaaatctctacacaccattctctaataccagcaccaccgacaccaccgccatgaccaacctgaggcaaccaaacattgggtgtttcatcaatgaaacaaccagctacccttctccaagcacgcagttctattatactgccccacattataattatatgccaacacatgaaaccctgcaaaatccatacacataccctatacaacaacctgatacatctcttgcattacctatgtacccattcatgcaaaatggaatccaggatggcctacaaagtccaatccatgggtcttcctttttgcaacaaaatgtgtcattaatgcagcaagagaaaaagtctatagatgcatataaaacgaagcttgcaaggattaagcgaaaacaggcacgccaaaagagcattagagccaacaaaaattcttcagcttcttcatcaactagttcaactcaagctgatgttaatgagagaaacaacgagggcaacagagatctgtacaagttctttaccccagatgataagagactgagagtgctactaaagaaggaattgaagaatagtgatgttggatctcttggaaggattgtccttccaaagagggaggctgaggaaaatcttccaattctcactgataaagaaggcattggaattgtgttaagagatttatattctatccaagagtggagcttcaagttcaagtactggtctaacaataagagcagaatgtatgttcttgaaaatacaggaaaatttgtaaggcaaaatgggctagagatgggagattcccttactctttacgaggatgaaagcaaacatctctatttctccataaaaaagacggaaaaaccgacggctgagcccttatacgaacaacattatgcaaaccaaaacaacaattacctgtacacaccaacaagttggcaggctagagatgaagaagaagcctctctagcactacttatagaacaacttaaacacaaagaacatcaagaagttaacaacctcatggctttggattgtgcttcttcttcttcatatatgcagaccgaggaagcacaaatggatttgtcgaagcagggaacatttaccctattgggggcagcatcaagtatgcaacattcatatccatggcatggtaacttggacgctgttaatgattgtcaactcaatgtggatgattattatggtggccttggtatgcttccggatcgattataa

SEQ ID NO: 19 - protein, 405 aa, Cephalotus follicularis
MENLYTPFSNTSTTDTTAMTNLRQPNIGCFINETTSYPSPSTQFYYTAPHYNYMPTHETLQNPYTYPIQQPDTSLALPMYPFMQNGIQDGLQSPIHGSSFLQQNVSLMQQEKKSIDAYKTKLARIKRKQARQKSIRANKNSSASSSTSSTQADVNERNNEGNRDLYKFFTPDDKRLRVLLKKELKNSDVGSLGRIVLPKREAEENLPILTDKEGIGIVLRDLYSIQEWSFKFKYWSNNKSRMYVLENTGKFVRQNGLEMGDSLTLYEDESKHLYFSIKKTEKPTAEPLYEQHYANQNNNYLYTPTSWQARDEEEASLALLIEQLKHKEHQEVNNLMALDCASSSSYMQTEEAQMDLSKQGTFTLLGAASSMQHSYPWHGNLDAVNDCQLNVDDYYGGLGMLPDRL

SEQ ID NO: 20 - DNA, 1092 bp, Tarenaya hassleriana
  source 1..1092: mol_type = other DNA
atggagaacttctcctcctcctctccaaatacccctcttttctcagtccatgatcattcatcgtgtcaaaacagcaatctgcaacagttcatgatgagccctaacccgtatgagtattcatacgcattccaaggtggtcaagttgatcaacagacggagtttcattcttttcccaacgcgaattcggcatttcctcacatctccatgccgattcagcagaacggatgcgaacccgtggtaccaacaccgagttcgctggttaactcgggcgggttggggcaaggactcggagggtttcttgatccgcagaagacaaagatagcgaggattaagcgaaaactcgcgaggcaaaggacacttagcttgtcaagaaacaactcttcttcgtcttcttcttctaataactctaattctaatcacagttcaaacgagtcggattcaaggagacaggtgacgtttaagggtcatccccataacttctcccaaggtgatcaatttcggttctgcggattgaataacaagaagcttagggttttgttggtgaaggagttgaagaacagtgatgtaggggcgcttgggaggattgtgcttccaaagagagaagcagaagcaaatcttccaacgctgtcggataaagaaggaatcaaaattgtgatgagagatgcggactctaaccaagcttggtctttcaagtacaagttctggtccaataataagagcagaatgtatgtcctcgagaacacaggggagtttgtgaagcaacatggactagagatgggggactcgtttacaatacacgaggacgaaagcaagaatctctattactcaataagaaaggcagagaagccgaaagaagcaacagaaaacgcaaaggaagaggagatgatagagacaacgggagaatacacaccaagagacgaagacgaagcttccttggctttgctcattgaacagctcaaggacaaggagcaacagctcgacatgagcttgtccgccatccatgacggagatccctcgcgtgctgctcacgtgactgatggtcttggtgcgtcaacccagtttcacagctatgctactagtttcccattctga

SEQ ID NO: 21 - protein, 363 aa, Tarenaya hassleriana
MENFSSSSPNTPLFSVHDHSSCQNSNLQQFMMSPNPYEYSYAFQGGQVDQQTEFHSFPNANSAFPHISMPIQQNGCEPVVPTPSSLVNSGGLGQGLGGFLDPQKTKIARIKRKLARQRTLSLSRNNSSSSSSSNNSNSNHSSNESDSRRQVTFKGHPHNFSQGDQFRFCGLNNKKLRVLLVKELKNSDVGALGRIVLPKREAEANLPTLSDKEGIKIVMRDADSNQAWSFKYKFWSNNKSRMYVLENTGEFVKQHGLEMGDSFTIHEDESKNLYYSIRKAEKPKEATENAKEEEMIETTGEYTPRDEDEASLALLIEQLKDKEQQLDMSLSAIHDGDPSRAAHVTDGLGASTQFHSYATSFPF

SEQ ID NO: 22 - DNA, 1104 bp, Juglans regia
  source 1..1104: mol_type = other DNA
atggacaactttttcacacccattcccacaaccaagacaaccaccagagccccttcaacctcttcacccttcgaaacctctctcgtgtattattttccaccccacgaaggtcatctgcaatttccccacgcatttcctttagatcaggttggtcagcatttggcgtacccggcatattctttcgtaattggggcgggtccgcaaccacatttgctgtcgaaagaacaagagagaaggcttgtggatgcttggacaaccaaggttgcgaggagtaagcgcaaaatggcacgccaaaggagcctcagttcaagcaaaaatcctgcttcctgggccacttcgactcaggtatatacccggggactgcccgtgtacggtgctgttgatgctgagacgcagaacagcaacatcaaaagagatccctacacgttcttcacgccggataagaagaggcttagagttttacttaaaaaggagttgaagaatagcgatgttgggtccttgggcaggattgtgctccctaagagagaagcagaagaaaacctcccaaccctctctgataaagatggaatccaaattgtgatccgagatgtgtattctaaccaagagtggagcgtgaaatacaagtattggtcaaataataagagcagaatgtatgttcttgaaaatacagtggattttgtgaggcaaaatgagctggtgattggagattccattactctttacgaggatgaatgcaagaatctcacaggtaggactgagcacgtgcgaaacaagcacagcttctttgggtatgtcaccattaaaaggggtgaaagaccagtagtagatcatcaagatctagaagatgatgatcagcagcagcagcaacgtcctgcatacagaaaagacatgatgatgatgagtaatagaaaccacatgatgatgagctacaaccaatattacaccccatttacatacgggaacagagatgaggaagaagcatatcttgcattactcatagagcaactcagacacaaggaagaacttgaagctaacagccttgcaactttgtctatcggatcaggtcatgctgcttcttcttcacataggcagcccctgatctaa

SEQ ID NO: 23 - protein, 367 aa, Juglans regia
MDNFFTPIPTTKTTTRAPSTSSPFETSLVYYFPPHEGHLQFPHAFPLDQVGQHLAYPAYSFVIGAGPQPHLLSKEQERRLVDAWTTKVARSKRKMARQRSLSSSKNPASWATSTQVYTRGLPVYGAVDAETQNSNIKRDPYTFFTPDKKRLRVLLKKELKNSDVGSLGRIVLPKREAEENLPTLSDKDGIQIVIRDVYSNQEWSVKYKYWSNNKSRMYVLENTVDFVRQNELVIGDSITLYEDECKNLTGRTEHVRNKHSFFGYVTIKRGERPVVDHQDLEDDDQQQQQRPAYRKDMMMMSNRNHMMMSYNQYYTPFTYGNRDEEEAYLALLIEQLRHKEELEANSLATLSIGSGHAASSSHRQPLI

SEQ ID NO: 24 - DNA, 1194 bp, Populus trichocarpa
  source 1..1194: mol_type = other DNA
atgggttgtcctcagaattcccagttctcttctatgtatggaccattgagctaccattcaacagtaccttcaaattccgtaactcagtcaaactgcgttccgccacagtactatatgccagcgccttgtgaaagtcgacaaaatccatacacgcattctctagagaagcctagccatcatacaccatctgcagatcctttccgtcttgccatggctggccaaaatgcttttgaaatgcaaagatcatgcattttgcagcctaatgcgttccaattagagcaagaaaggagagctcttgatgcatacaagaccaaggtggcaaggtgtaagagaaaacttgcacgccaaagaagccttagtaaaacttctattggggctagttcaactcaaataatggatgcaaggagattggctttgcatggtgctagtacttgtggccaaagaagcaggagcaataccagcaaagatctttacgagttcttaacacctgataacaagagactcagagcggtgcttaggaaggacttgaagaatagtgatgttgggtctcttggcagaattgttcttccaaagagagaagtagaggaaaatcttcctgtcctaaatgataaagaaggaatcctactttttctcagagatgtatactctaaccaagagtgggccttaaagttcaagttttggtctaacaacaaaagtaggatgtatgttcttgaaaatacaggagaattcgtaaagcaaaatgggctggagactggagatttccttactctttacgaggatgagagcaagaatctctatttctctatcacaaaagtgggaaaaccagcatcggtgccatctcaaacaccacagcctattaaccacaactctaactgcctatacaccccgcatatgtaccaagctagggatgaagaaaaatcatctttagcactacttatagaacaacttgaccaaaaggagcaagaagaagctaacagcctcgtggctgtgcctccggactctgcttacacaaacaacgaactaaccaacaatcctttcaacaactcaagtacctattcccaaccagcatcttcagctatgcaaccttcttcaccaaatggtaaaatgaaggcggtagatgattctcatgtcgatgactgctatactggtctcggtgtgcttcctgatgtctaccgatttaatttttcgctatga

SEQ ID NO: 25 - protein, 397 aa, Populus trichocarpa
MGCPQNSQFSSMYGPLSYHSTVPSNSVTQSNCVPPQYYMPAPCESRQNPYTHSLEKPSHHTPSADPFRLAMAGQNAFEMQRSCILQPNAFQLEQERRALDAYKTKVARCKRKLARQRSLSKTSIGASSTQIMDARRLALHGASTCGQRSRSNTSKDLYEFLTPDNKRLRAVLRKDLKNSDVGSLGRIVLPKREVEENLPVLNDKEGILLFLRDVYSNQEWALKFKFWSNNKSRMYVLENTGEFVKQNGLETGDFLTLYEDESKNLYFSITKVGKPASVPSQTPQPINHNSNCLYTPHMYQARDEEKSSLALLIEQLDQKEQEEANSLVAVPPDSAYTNNELTNNPFNNSSTYSQPASSAMQPSSPNGKMKAVDDSHVDDCYTGLGVLPDVYRFNFSL

SEQ ID NO: 26 - DNA, 1239 bp, Aquilegia coerulea
  source 1..1239: mol_type = other DNA
atggattctaacaaacctattgccacttccaacaccaccactagtactaccagccctacttcctctgcaactcccaatcaagaagtgaataccacaatcaatcacgtagatcctcttccacctcatcttcttcttcctcctcctcataattattcttcaaattgtcccaatgttgctccagaggcacaaatctttccatcacattcttacccctaccaaggtctacattaccaaccaaatcaatctcaatcacatccattggtcgtacctccatactatctcgttccatccatctcccccgcctataaattagatgattcatcaagaattttaccaatgtatcttcaatataaatgggacccatcggtctcaaaggaggctagaaaccgaagaaggatggctaggcaaatgagctcaagctcatgcagaacctctgaaggttcttcatccagtgggtcttcaagcattcttcctactacaactgaaaatgaggataacgacttgatagctcatgatggcaaaaagctcaaatttatattgcggaaagagctaaaaagaactgattgtggctcccaaggaaggattatcctcccaaagaaagactcagaggtgaatcttccatccttgagtgcgagggagggaatccaactaacgttgagagaggtgggttccacgactatttggtgcttcagatacagatattggcctaacaataatagtcgaatgtacttattcgaaaatacaagggattatgtaaagcaaaatgagcttcagattggggataatattatcctctacaaagatgaagggaatgagctttacatttcgtataaaaaggccagtgtctctatctcagcacctacaaataatgagggaggcagtagtgaggtggaaaggtcactccaaggtaaaccttcagaggatagcttcagcgagtttggtcacagttcatcgaacatggttgacgaagaagatacctggtacgaacaagcaggtattctactagatgaaccaacgtgtgctaacgacataatgatagatgaatcacaacaacttgaagattcatattgcatggatgcacacaagggttatgacttcagagacttcgaacaactagcagattttaaggactgtgacttctcgttatataataaagatgacgatctgatgcagctgaaccttgatgatgaccaaaataacaaagacaagggtggaggtagttcatccaatgacaactga

SEQ ID NO: 27 - protein, 412 aa, Aquilegia coerulea
MDSNKPIATSNTTTSTTSPTSSATPNQEVNTTINHVDPLPPHLLLPPPHNYSSNCPNVAPEAQIFPSHSYPYQGLHYQPNQSQSHPLVVPPYYLVPSISPAYKLDDSSRILPMYLQYKWDPSVSKEARNRRRMARQMSSSSCRTSEGSSSSGSSSILPTTTENEDNDLIAHDGKKLKFILRKELKRTDCGSQGRIILPKKDSEVNLPSLSAREGIQLTLREVGSTTIWCFRYRYWPNNNSRMYLFENTRDYVKQNELQIGDNIILYKDEGNELYISYKKASVSISAPTNNEGGSSEVERSLQGKPSEDSFSEFGHSSSNMVDEEDTWYEQAGILLDEPTCANDIMIDESQQLEDSYCMDAHKGYDFRDFEQLADFKDCDFSLYNKDDDLMQLNLDDDQNNKDKGGGSSSNDN

SEQ ID NO: 28 - DNA, 2364 bp, Arabidopsis thaliana
  source 1..2364: mol_type = other DNA
atgagctcctccataaacaattggttgggtttctccctctctcctcaacaacagcagcagcagcaacagtcttctgaccattctcaatctcagcagcaccaccattctggttttgcctcctccttcaacaacaacaatgatctttcagtctcaggagaaggaggagactgctatatggggcttggtggtggcgattctcaagccaccaccctcagcaggccagacggtccttttgggatcttagaagcgctcaacagacctcatcaaaaccaagattggagcgtgaagagcttgagcgtgggcatcatcaactccgactcgagttgttacggtggtggtggcggtggaacgaccaccaatgttatagcagaacggcagcatcaagatggagtaggaagaccaaagcttgaggatttcctaggcggtgcctccctcggaggaggaggaggagggagcaccacccactatactggtggtggtgagaacaacaacaacaataacaacatgctattctcaatggactgctccttgcagcttccttccaatgggaatgggaacaactcatcgtcgtcgtcgatgttagggctgtcgatgataaagacttggttgaggaaccagccacattcatcttccaccgagaatgatggaggaggagcagcggtaagcaacactcatcagacactgtcactctccatgggcactggtggtggcagcacacatcatcagcagcagcaaggccccattcaaatccctttgcctctcccggcgcctatggcagtgcccggtgagagttcgtcctccgacaacaatgtgaagcagcagaagcctcctcctctcgaccaggcgggtgccattgagccagtgccaaggaagtctatcgatacatttggccaacgaacttccatttaccgtggtgtcacaaggcataggtggactgggagatatgaagctcatttgtgggacaacagttgcaggagagaagggcaaacccgcaagggtcgacaaggtggttatgacaaagaagaaaaagcggcaagggcttatgatttggcagctctaaagtactggggaaccactaccaccacaaatttcccgataagtaactatgagaaggaggtagaagagatgaaacatatgaccaggcaggagtatgtggcatccctcaggagaaagagcagcggtttttccaggggagcatcaatttaccgtggtgttacaaggcaccatcagcatggaagatggcaggcaaggatagggagggtagcagggaacaaagatctttacttggggaccttcagcactcaagaggaggcagcggaggcatacgacattgctgcgataaagttcaggggtttgaatgcagtgacaaacttcgacatgagcagatacgatgtgaacagcattatggagagcagcactttgcccattggaggagctgccaaaagattgaaggagtcatctgatcaaaccaccaaccaccccatgccattgccattgccattgccattgggggagagcaccatgcccatgcccatggccatgcctatggccatgccattgcccatgagcatgagaagagcaacggatgaagacaacattatctccatggctaatgctgctgcttcacacatgacgacaaacgagggcatcgctactgctgccggttatgggtggcccatattaggatttcatcatcagccattgaccacccaacactttccctactcccactcccaaaacccccagcagaggggaggttggtgcaaatcagaaaggcagcagcacgaggaaagcaacagcaatagcaatagcagtaacatcatgcaagaagcccaccaactccatcttgggggtggcagcaataatacccacaacttcctccaacagcaagcatctgtgttacacaacctcattagtttagattctccgtcgtccttcgatcagacttccgcctcgaattccatcatatacacaggtagcagcggctatcaattgggtgggcccattggcaccactataataccaaccggtgtcaatccccatgttgcagatcatcatcaaggaagccttggagaaggagaaaccaagcaattgggtttttatgagaatggttatttgtcagttgctgccgacccttactcagcaggggcaaggaatgtttactatctgcagcagaaccagtcaaaggcaaacaacaatggcaactatgatgatgatagccaccatcagactaataacaacaacatcagtagttggatgatcccaacaacttccctccaggctttggcacccaccagatcatcatcaaaccttgcagcagtttgccatgccactccgattttctcggtgtggaacgacacatga

SEQ ID NO: 29 - protein, 787 aa, Arabidopsis thaliana
MSSSINNWLGFSLSPQQQQQQQQSSDHSQSQQHHHSGFASSFNNNNDLSVSGEGGDCYMGLGGGDSQATTLSRPDGPFGILEALNRPHQNQDWSVKSLSVGIINSDSSCYGGGGGGTTTNVIAERQHQDGVGRPKLEDFLGGASLGGGGGGSTTHYTGGGENNNNNNNMLFSMDCSLQLPSNGNGNNSSSSSMLGLSMIKTWLRNQPHSSSTENDGGGAAVSNTHQTLSLSMGTGGGSTHHQQQQGPIQIPLPLPAPMAVPGESSSSDNNVKQQKPPPLDQAGAIEPVPRKSIDTFGQRTSIYRGVTRHRWTGRYEAHLWDNSCRREGQTRKGRQGGYDKEEKAARAYDLAALKYWGTTTTTNFPISNYEKEVEEMKHMTRQEYVASLRRKSSGFSRGASIYRGVTRHHQHGRWQARIGRVAGNKDLYLGTFSTQEEAAEAYDIAAIKFRGLNAVTNFDMSRYDVNSIMESSTLPIGGAAKRLKESSDQTTNHPMPLPLPLPLGESTMPMPMAMPMAMPLPMSMRRATDEDNIISMANAAASHMTTNEGIATAAGYGWPILGFHHQPLTTQHFPYSHSQNPQQRGGWCKSERQQHEESNSNSNSSNIMQEAHQLHLGGGSNNTHNFLQQQASVLHNLISLDSPSSFDQTSASNSIIYTGSSGYQLGGPIGTTIIPTGVNPHVADHHQGSLGEGETKQLGFYENGYLSVAADPYSAGARNVYYLQQNQSKANNNGNYDDDSHHQTNNNNISSWMIPTTSLQALAPTRSSSNLAAVCHATPIFSVWNDT

SEQ ID NO: 30 - DNA, 1788 bp, Helianthus annuus
  source 1..1788: mol_type = other DNA
atgaacaactggttaggtttctcactctcacctcaagatcagcaacaaatggtggattcacggtcatctatggttgataacctcaactcggatagcatctccggcaccggagagtgttttgatttgggctctaacgctgttcactcctccataccttctctcaaccttcctcctccgtttggtttactcgaaccccaccatgattggaacatgaagggcgggagttcagagttgtccatgttgatgtctggtggtggttcatgcaatgaaaaccctgaaatccaaccaccaaagtttgaaaactttctaaatattggtaacaccggaagctacatgtaccaacttccgccggaggtcaccaccaccagaggaaactctaacactgccactagtagcattggtctttccatgataaaaaactggctcagaaacaacccaactccatctaatcaaaacgccgccaccggacctcaagaaaccgccccaatgagtgtgttttccagtggtgctagaaccaatgagctttcactttcgatgggcaccggtggagatgataataaacgccagcaaatggaggtggcaaccaccgtagatagtgaaaacgctgctgccgtgcccaggaaatccattgatacatttggacaaagaacttctatttaccgtggtgttacaagacacagatggacgggaagatacgaagctcatttatgggataatagttgcagacgagaaggacaagttcgtaaaggaagacaagtttatttgggaggctatgataaggaggacaaagcagcaagagcatacgatctagcggcgctaaaatattggggcaccacgaccactacaaattttccgattagtaactacgagacggagatcgaggagatgaagaacatgacgagacaagaatatgttgcctctcttagaaggaaaagtagcggtttttcacgaggtgcttccatctatagaggtgtgacaaggcaccatcaacatggaagatggcaagcaagaattgggagagttgcagggaacaaagatctttatttgggaacttttggaacacaagaagaagcggccgaggcgtacgacatagccgcgatcaaatttcgcggattgaatgcagtcacaaactttgaaatcaacagatatgatgtgaagtgcatacttgagagcaccaccttaccaataggtggtgcggccaaacgtctcaaagacgcagaaaaagcagcagccaccgagttactgatcagggctgctaaagatgaccacttgaccacccatgaaggctatgccaccatgacatggccgagctccatcgactaccagcaccaccaagcaatggctcaagctcaaacccaaccatttagcatgcaatacccacaacactataatacctcgaaccaacaacggttatggtgcaaacaagaacaacagccagatggtcaaagcttccaagatcttcatcagttctttcaaccatcaccaaattcttctgtccttcacaatctcatgagcttggattcgtcttcaatggaacatagttatagcagcggaggaggaggaggaggaggtgatgatctggtgatgggatatggtggcaccggcagtggttatatgatggctaatactacaaacactgattcgtctcaacatggtgatggaagcttgtactaccatcaccaaggttccggttatggtggttgggtgccaaccgctataccaacgctgccaccacaaagtgccaacttggtggtggctcccaactttactgcttggaatgactcataa

SEQ ID NO: 31 - protein, 595 aa, Helianthus annuus
MNNWLGFSLSPQDQQQMVDSRSSMVDNLNSDSISGTGECFDLGSNAVHSSIPSLNLPPPFGLLEPHHDWNMKGGSSELSMLMSGGGSCNENPEIQPPKFENFLNIGNTGSYMYQLPPEVTTTRGNSNTATSSIGLSMIKNWLRNNPTPSNQNAATGPQETAPMSVFSSGARTNELSLSMGTGGDDNKRQQMEVATTVDSENAAAVPRKSIDTFGQRTSIYRGVTRHRWTGRYEAHLWDNSCRREGQVRKGRQVYLGGYDKEDKAARAYDLAALKYWGTTTTTNFPISNYETEIEEMKNMTRQEYVASLRRKSSGFSRGASIYRGVTRHHQHGRWQARIGRVAGNKDLYLGTFGTQEEAAEAYDIAAIKFRGLNAVTNFEINRYDVKCILESTTLPIGGAAKRLKDAEKAAATELLIRAAKDDHLTTHEGYATMTWPSSIDYQHHQAMAQAQTQPFSMQYPQHYNTSNQQRLWCKQEQQPDGQSFQDLHQFFQPSPNSSVLHNLMSLDSSSMEHSYSSGGGGGGGDDLVMGYGGTGSGYMMANTTNTDSSQHGDGSLYYHHQGSGYGGWVPTAIPTLPPQSANLVVAPNFTAWNDS

SEQ ID NO: 32 - DNA, 1572 bp, Amaranthus hypochondriacus
  source 1..1572: mol_type = other DNA
atgatcaaaacatggttaagatcaaacaatccttctcaaacaaatatcatcgacggcagcggtggcagtggcggtggcggtggcggtagtggtggtggcgatagtgttgctaccatatcaaatgcacaaactttgtcgctttcaatgggtacaagtggtgggacccatcactctaactccgctttgtcgctaaacgcggcaagcggaattagtggtggtagtgaaatggagagtaaacaacaacaacaaaatgatacaacaacggccacccttgatgctcaaagtggtgcactagaaagtgttccaaggaaatctattgatacttttggacaaagaacgtccatttaccgtggtgtaacaagacataggtggactgggagatatgaagctcatctgtgggataatagttgtaggagagaagggcaaactcgtaagggcaggcaaggtggttatgacaaagaagaaaaagcggctagagcttatgatttggctgcaattaaatactggggttctaccactactaccaactttcctattagtgattacacgaaagaaattgaggatatgaagcctatgacacgacaagaatatgttgcatctttacgaaggaaaagtagtggattttctcgtggtgcatcaatttaccgaggagtaacaaggcatcaccagcatggtcgttggcaagcaaggataggcagagttgcaggcaacaaagacctttaccttggcactttcagcacacaggaagaagcagcagaagcatatgacatagcagcaataaaattcagaggattaaatgcagtaactaactttgagataaacagatatgatgtgaaagccatacttgatagtgccactcttcccattggaggtgctgctaaaaggttaaaagatgttgaagatcatttaacaccttcttctgataaacaaatcattaccccaattaatactcatgatcattcccatctttcttcttatgctaatgggattcccaactttcattcctggcctggaattgcattcccacaaccccagcaaccaatcacaatgcacaattacccttatgctaattcccatcaacaaagggtatggtgtaagcaagaagtacaggattgtaatgatcatgattatcagaatcatcttaatcaaattcagatcaatagtgggtgtgggacccataacttttttcagatgcataatttgatgggtttggagaattcttcttctagtatggaacagagttctgggtctaactctcttctttacgggaatgggaatggtaatgggaatgatggtattggtgggtatggattccctcctgggaatgggatgtctacagttgtagctgaagatgggaatggaaatgggaatgggaatgagaattattattattactcacaagggaatggtagtgtaagaggaaatgttgggacttatgatcaaggatcagcttgtaacaactgggtcccaactgctatacctgcactcggcccgagaacgacccatctggctgctgttggtggtcatgaaggaggaggaatccctacttttaccgtctggaatgacacataa

SEQ ID NO: 33 - protein, 523 aa, Amaranthus hypochondriacus
MIKTWLRSNNPSQTNIIDGSGGSGGGGGGSGGGDSVATISNAQTLSLSMGTSGGTHHSNSALSLNAASGISGGSEMESKQQQQNDTTTATLDAQSGALESVPRKSIDTFGQRTSIYRGVTRHRWTGRYEAHLWDNSCRREGQTRKGRQGGYDKEEKAARAYDLAAIKYWGSTTTTNFPISDYTKEIEDMKPMTRQEYVASLRRKSSGFSRGASIYRGVTRHHQHGRWQARIGRVAGNKDLYLGTFSTQEEAAEAYDIAAIKFRGLNAVTNFEINRYDVKAILDSATLPIGGAAKRLKDVEDHLTPSSDKQIITPINTHDHSHLSSYANGIPNFHSWPGIAFPQPQQPITMHNYPYANSHQQRVWCKQEVQDCNDHDYQNHLNQIQINSGCGTHNFFQMHNLMGLENSSSSMEQSSGSNSLLYGNGNGNGNDGIGGYGFPPGNGMSTVVAEDGNGNGNGNENYYYYSQGNGSVRGNVGTYDQGSACNNWVPTAIPALGPRTTHLAAVGGHEGGGIPTFTVWNDT

SEQ ID NO: 34 - DNA, 1953 bp, Cucumis sativus
  source 1..1953: mol_type = other DNA
atgggttcaatcaattggcttggcttctctctttcccctcaacaccaaccctccgatcacgctcacgctgccgccgctaccgcccccttcacctccgatcaaatctctccctccgacgtctccgccgtctgctttgacaaccttccttcttcctatgctgtttatgaacactctcaagattggaataatatgaagggtttagggtcgacacaaagttcagatttttctgctttaatcgaaagccaacaccaaccaaagcttgaaaatttcctaggccaccactctttcactgaccacgaccacgaccacgccaccgccgccgtctacaccaatgcctccgccaactacatcttccaaaattcttctttggaccttccatcggaggctgccggctgtggcggtggaagacccagcgccggtaacggaaacgccaacaatactacctctattggtttgtcgatgattaagacgtggttgaggaatcaaccagcgccgcctcaagtggtggccaaaggcggcggagatcacgacggtagtgcagttggaataagtaatcatctcacgaccgcacatactttatcactgtcgatgaacaccggtcccccaccgccatcccagtcttcgtccggctcggcggctctaccacttctcacggccagcggcggtgaaagctcctcctccgataacaaacaagggaaatcatcaggtgcctcgatcgacgctgaaaatggtgccgttgaagctgccccgagaaaatctgtcgatacatttggacaaagaacttccatctaccgcggtgtaaccaggcatagatggacaggaagatatgaagctcatctttgggacaatagttgtagacgagaaggacagactagaaaaggaagacaagtctatttgggaggttatgacaaagaagaaaaggctgcaagagcttacgatttagcagcacttaaatattggggtaccacaaccacaacaaattttccaattagtgattacgaaaaagagttggaagacatgaagcacatgacccggcaagagtttgttgcgtctctaagaaggaagagtagtgggttttctcgaggagcttccatttaccgtggagttacaagacaccaccagcatgggagatggcaagcaagaatagggagagttgcggggaataaagatctctacctgggaactttcggtacgcaagaagaagcagcggaagcatatgacatagcagctataaagtttagaggattaaatgcggtgaccaactttgatatgagccgttacgatgtgaaagccattcttgaaagtaacacattacccatcggcggggctgccaagcgcttaaaggacattcaccattctgacatacctctcgacccccaaagagcagaagacaacataacttcacacctctctgatggaataataaatattacatccaccacgactccttatagtggcggtggatggcccactatcgccttccaccatcaccatccttacgctttccactacccctatgcgactccgcaacaacgaatgtggtgcaaacaagaacaggatgctactgctatcgccgccgacaactttttcccacatggtggggctatggactcaatggaccatagctcggggtcttacagcagtgccaatggagatgggtacaatggaaacttcttgattccgatgggggcggaaggaagcagcagcaacaatgggggattaggagacattggagaagtgaatttatttggtggttgttctaatgtggatgatccattccatgcaacaagaacgtccaatttgtataattatcacaactcctctcatcagctgcaaccgccacctccgccgtcggggctgcaaggctctaactgcaacaactggcttcctccggcaaggtcggtttgtcaggggggagctcccccacctttcaccatttggaatgacacttag

SEQ ID NO: 35 - protein, 650 aa, Cucumis sativus
MGSINWLGFSLSPQHQPSDHAHAAAATAPFTSDQISPSDVSAVCFDNLPSSYAVYEHSQDWNNMKGLGSTQSSDFSALIESQHQPKLENFLGHHSFTDHDHDHATAAVYTNASANYIFQNSSLDLPSEAAGCGGGRPSAGNGNANNTTSIGLSMIKTWLRNQPAPPQVVAKGGGDHDGSAVGISNHLTTAHTLSLSMNTGPPPPSQSSSGSAALPLLTASGGESSSSDNKQGKSSGASIDAENGAVEAAPRKSVDTFGQRTSIYRGVTRHRWTGRYEAHLWDNSCRREGQTRKGRQVYLGGYDKEEKAARAYDLAALKYWGTTTTTNFPISDYEKELEDMKHMTRQEFVASLRRKSSGFSRGASIYRGVTRHHQHGRWQARIGRVAGNKDLYLGTFGTQEEAAEAYDIAAIKFRGLNAVTNFDMSRYDVKAILESNTLPIGGAAKRLKDIHHSDIPLDPQRAEDNITSHLSDGIINITSTTTPYSGGGWPTIAFHHHHPYAFHYPYATPQQRMWCKQEQDATAIAADNFFPHGGAMDSMDHSSGSYSSANGDGYNGNFLIPMGAEGSSSNNGGLGDIGEVNLFGGCSNVDDPFHATRTSNLYNYHNSSHQLQPPPPPSGLQGSNCNNWLPPARSVCQGGAPPPFTIWNDT

SEQ ID NO: 36 - DNA, 1782 bp, Tarenaya hassleriana
  source 1..1782: mol_type = other DNA
atgactcacatgaacaactggttgggtttctcatctctctctcctcgtgatgatgatcaagatcaaacccaccgtggccacggcttctcctccgccgtggatgtccccggagattgcttcgatctcgccgcagacgagtcctctgccgccgtcgccgtgcccgcgattccttccccttttgccctcctcgaccccttcaccagagacaacaatagtcactcccgagagtggaacatcaatggcggtgcatgcaatagtcaaaatggtccgaagcttgagaacttctttggtctcaaccaaaccgccgtttacgacagcattaacatcaacgaggataacgccataaactatggaaactgcggcggaggcggtggctcgtttgggctttcaatggtgaagacttggctgcgtgaccaaccggcgaattatgctgagaacaacgttgacgggcatggcggtgcacgaggcttgtctctttctatgagctcgtcttccgacaacaaaaataacaacaccgataatgatatgaaacacgatagttgtgatagtgctgtccaagagaagacgttgtcagagaccactgttggtttagaagccgtgccgaggaaagctgtcgacagtttcggacagaggacgtctatataccgcggtgtaacaaggcacagatggacgggtagatacgaggcacatttatgggacaacagttccaaaagagaaggccaaactcggaagggaagacaagtttatctaggaggttatgacaaagaagaaaaagctgctagggcttacgatttagccgcacttaaatattggggcaccacaacgactaccaacttcccgataggtgattatgagaaagagatcgaagagatgaagcacatgacaagacaggagtatgttgcatccttgaggaggaagagcagtgggttctctcgtggtgcgtccatgtatcgcggagtaacaaggcatcatcagcacgggaggtggcaagcaaggattggtagagtcgcgggcaacaaagatctctacttgggaactttcagcacgcaagaagaggccgcggaggcgtacgacatagcggccatcagattccggggattgaacgcagtgacgaacttcgacatgaacagatacaacgttaaggccattctcgagagcccgactcttcccattgggagcgcttctaaacgactcagagaaagtaccactgaacagatgacaattgcgagtaccgtttcagacgaagaaagttccctcaacactgcaagtggagtcaacaacctttggccttccatctcgtttcatctccctaatcaaggacagcttctgagcagctcgttgccaagatacgatgtcggaatcggacagggccgagattggtgccaccaagaacaggattccgagcttaagccgagccacgggtttcagcatttcttcgatagctcgcccagtctcgtgatgggttcaagcgattcagtgactatctgtggacgtgaggacgggtttggtggctaccaaggagctgggtacggtggcaggaacagcggattcgttgtccctatggccacatcggttctttccaacgagaacgtgatgatcaatggcagtgtatacagcgtgcctgacgttacttgcgatgcaagaaacgcgtattgccttgcacagcaagcggcatcgtcggagagggatttgccgatgggggttcctaatgttgcagccgttggttctaacatgtacagtggaggggcttcatcgacgttcactgtttggagagacacatag

SEQ ID NO: 37 - protein, 593 aa, Tarenaya hassleriana
MTHMNNWLGFSSLSPRDDDQDQTHRGHGFSSAVDVPGDCFDLAADESSAAVAVPAIPSPFALLDPFTRDNNSHSREWNINGGACNSQNGPKLENFFGLNQTAVYDSININEDNAINYGNCGGGGGSFGLSMVKTWLRDQPANYAENNVDGHGGARGLSLSMSSSSDNKNNNTDNDMKHDSCDSAVQEKTLSETTVGLEAVPRKAVDSFGQRTSIYRGVTRHRWTGRYEAHLWDNSSKREGQTRKGRQVYLGGYDKEEKAARAYDLAALKYWGTTTTTNFPIGDYEKEIEEMKHMTRQEYVASLRRKSSGFSRGASMYRGVTRHHQHGRWQARIGRVAGNKDLYLGTFSTQEEAAEAYDIAAIRFRGLNAVTNFDMNRYNVKAILESPTLPIGSASKRLRESTTEQMTIASTVSDEESSLNTASGVNNLWPSISFHLPNQGQLLSSSLPRYDVGIGQGRDWCHQEQDSELKPSHGFQHFFDSSPSLVMGSSDSVTICGREDGFGGYQGAGYGGRNSGFVVPMATSVLSNENVMINGSVYSVPDVTCDARNAYCLAQQAASSERDLPMGVPNVAAVGSNMYSGGASSTFTVWRDT

SEQ ID NO: 38 - DNA, 2235 bp, Juglans regia
  source 1..2235: mol_type = other DNA
atgcatgaactagttcctcatctccctttgctcagcggccaatcaatattattttcacccacaagtccttcaaaacgatatatggcttccatgaactggttgggtttctctctttcccctcaagaactcccatcgcagcctgctgatcaagatcactctcaaaacaccgagcctcgccttgccttcaactctgacgacatctccggtacagatgtctcaggcgactgcttcgatctcacttctgactcgactgctccatccctcaacctccctccacctttcggcatacttgaagcattcaatagaaacaatcaagctcaagattggaatatgaagggtttagggatgaactcgagtactaactacaagaccaccacagacctatctatgctaatgggtaacccgtgcagtagccaaaaccttgaaaaccaaaaccagccaaagctcgagaacttcctcggccgacactcgtttgggcatcacgaaaacgccgcatacaataacaatggtgactacatgttcaataactgttccttacagttaccctcagaagcagcaaacggcgtggcaagcagtggcgctggcggttgtagtggtagcatccacaacaataactctattggtttgtccatgatcaagacctggctgaggaaccaaccggcaccaacgccggctcagcaggataacaagaacgatggtgcgggtgcaagtagtggtggaaataacggaagcctagcaagtgcacaaaccttgtcgctctcaatgagcaccggatcacagtctagctcgacactgccacttcttacggcgagtactactggtggtgcaagtggtggagagagttcctcatcggataataagctgcagaagacaacacctgggcttgatagccaaactggtgctatcgaggcagtgccaagaaaatccattgatacatttggacagaggacttctatataccgtggtgtaacaaggcacagatggacgggcagatatgaggctcatctttgggataatagttgtagaagagagggacaaactcgtaaaggaagacaagtttatttgggaggttacgacaaagaagaaaaggcagcaagagcttatgatttagcagcactgaaatactggggtacaactaccactacaaattttcctattagcaactacgagaaagagttggaagaaatgaagcacatgaccaggcaagagtatgttgcgtctttgcgaaggaaaagtagtgggttttcacggggtgcatctatttatagaggagtgacaagacaccaccagcatggacgatggcaagcaagaataggaagagttgcagggaacaaagacctttacttgggaacctttagcacccaagaggaagcagcagaggcctatgacattgctgccatcaagttccgaggactgaatgcagtaacaaactttgacatgagcagatatgacgttaaaagcatactggagagcagcactttgcccattggtggggctgcgaagcgtttaaaggatgttgaacaggctgagatgactgtggacgggcaaagaacagatgatgaaaacatcacttctcggttaaccgatggaatcaatggctatggtagtgctgtacaccatggctggcctactcttgcattccaacaagctcaaccttatagcatgcactacccatatgggcaaagggtttggtgtaagcaagaacaagattctgaagctcccaacaactttcaagattttcatcagttacaactgggaaatacccacaatttctttcagcctaatgttttgcacaacctaatgggtatggactcagcttctatggagcatagttctggctctaactctgtcatttatggcagtcaaggaggcgcagatggtaatggtggagttgggtatggaagcaatggtggctatgcaatccctatgagtacgataattaccaacgacagcaaccaaagtcaaggcaatcacggaacttttggtgatagcgaggtgaaggcacttggttatgaaaacgtgtttgcctcagcagatccttaccaagccaggaacttgtattatctttcacagcattcaacggcaagtgtggttaagaccactaccgcttatgatcagggctcgatgtgcaacaattgggtgccaactgctgtgccaactctcgcgccgaggtcaagcaatatggctgtttgccatgcagcagctccaactttcacggtgtggaatgatacctaa

SEQ ID NO: 39 - protein, 744 aa, Juglans regia
MHELVPHLPLLSGQSILFSPTSPSKRYMASMNWLGFSLSPQELPSQPADQDHSQNTEPRLAFNSDDISGTDVSGDCFDLTSDSTAPSLNLPPPFGILEAFNRNNQAQDWNMKGLGMNSSTNYKTTTDLSMLMGNPCSSQNLENQNQPKLENFLGRHSFGHHENAAYNNNGDYMFNNCSLQLPSEAANGVASSGAGGCSGSIHNNNSIGLSMIKTWLRNQPAPTPAQQDNKNDGAGASSGGNNGSLASAQTLSLSMSTGSQSSSTLPLLTASTTGGASGGESSSSDNKLQKTTPGLDSQTGAIEAVPRKSIDTFGQRTSIYRGVTRHRWTGRYEAHLWDNSCRREGQTRKGRQVYLGGYDKEEKAARAYDLAALKYWGTTTTTNFPISNYEKELEEMKHMTRQEYVASLRRKSSGFSRGASIYRGVTRHHQHGRWQARIGRVAGNKDLYLGTFSTQEEAAEAYDIAAIKFRGLNAVTNFDMSRYDVKSILESSTLPIGGAAKRLKDVEQAEMTVDGQRTDDENITSRLTDGINGYGSAVHHGWPTLAFQQAQPYSMHYPYGQRVWCKQEQDSEAPNNFQDFHQLQLGNTHNFFQPNVLHNLMGMDSASMEHSSGSNSVIYGSQGGADGNGGVGYGSNGGYAIPMSTIITNDSNQSQGNHGTFGDSEVKALGYENVFASADPYQARNLYYLSQHSTASVVKTTTAYDQGSMCNNWVPTAVPTLAPRSSNMAVCHAAAPTFTVWNDT

SEQ ID NO: 40 - DNA, 2166 bp, Populus trichocarpa
  source 1..2166: mol_type = other DNA
atggcctccacgaataactggttaggtttctctctatcccctcaagaacttccatcatcacagtctgatcaccacgatcaccctcaaaacacagactctcgccttcgtttccactcagatgaaatctctggcaccgatgtctccggggagagctttgatcttacttctgactccaccgctccttctctcaacctccctgcctcttttggtatacttgaagcctttagaaacaatcaatctcaagattggaataatatgaagcgttcaggcatcaatgaagacaccagctacaataccacctcagacgtcccgatattcatgggttcttcatgcaatagccagaacattgatcaaaatcaagaacctaagcttgagaacttccttggcggccattcttttggcaatcatgaacacaaacttaatgtctgcagcaccatgtatggttccactggacactatatgttccacaactgttccttgcaactcccatctgaggatgcatcaaatgaaagaacaagcagtaatggaggggctgatactagcatcaacaataataacaccaatagttcgattggtttatctatgattaagacttggctaaagaaccagccggcaccgacacaacaagacaccaataataagagcaatggcggtgcacaaagcttgtccctttcgatgagtactgggtcacaatcaggttctgatttgccacttctagcagtaaatggaggaggaaatagaactagaggagagcagagttcttctgataataacaaacaacaaaagaccacaccgagtcttgatagccaaaccggtgccatcgaagtggtgccaaggaaatctattgatacttttggccaaagaacttctatataccgtggtgtaacaagacatagatggactggtagatatgaagctcatctatgggacaatagttgtagaagagaaggacaaactcgcaagggaaggcaagtttatttgggtggttatgacaaagaagataaggcagctagagcttatgatttagcagcattgaaatactggggcactaccaccacaacaaattttccaatgagcaactacgaaaaagagatagaagaaatgaagcacatgactaggcaggagcatgttgcgtctcttcgaaggaaaagtagtgggttttctcggggggcttccatttatcgaggtgtaactagacatcatcaacatggaagatggcaagcaaggattggcagagttgcagggaacaaagacctttacttgggaactttcagcacccaagaagaagcagcagaagcctatgacattgcagccataaaattccgtggactgaatgcggtgaccaacttcgacatgaacagatatgatgttaacagcataatggagagcagcacattgccaattgggggtgcggctaagcggttgaaagaggcggagcatgctgaaattacaacgcgtgtgcaaagaacagatgatcatgacagcacgagttcacaactcaccgatgggattagcaactatggtactgctgcacaccatggctggcctactattgcatttcaacaagctcaggcttttaccatgcactatccctatggccagaggctttggtgcaagcaagaacaggactctgacaatcatagctttcaagagcttcatcaattacaattgggaaacacgcaaaatttccttcagccttcggttctacataacgtcatgagcatggaatcgtcttcaatggaacacagctctggctctgattctgttatgtatagcagtggaggccacgatggtaccggtactggaaccaacggaagctatcagggaattggctatggaagcaatactgggtatgccatcccaatggctacagttatagctaatgatgtaaacacccaagatcaaggaaatggttatggagatggagaggtgaaggctcttggctatgaaaatatgttttcctcgtcggacccttatcatgctagaaatttgtactatctttcgcagcaatcatctgccggtgtgatcaaggctagtgcatatgatcagggttcaacatgtaacaattggttgccaacagcagttcctactattgcagcgaggtctaacaatatggctgtttgccatggagctccaactttcacagtatggaacgagagtacataa

SEQ ID NO: 41 - protein, 721 aa, Populus trichocarpa
MASTNNWLGFSLSPQELPSSQSDHHDHPQNTDSRLRFHSDEISGTDVSGESFDLTSDSTAPSLNLPASFGILEAFRNNQSQDWNNMKRSGINEDTSYNTTSDVPIFMGSSCNSQNIDQNQEPKLENFLGGHSFGNHEHKLNVCSTMYGSTGHYMFHNCSLQLPSEDASNERTSSNGGADTSINNNNTNSSIGLSMIKTWLKNQPAPTQQDTNNKSNGGAQSLSLSMSTGSQSGSDLPLLAVNGGGNRTRGEQSSSDNNKQQKTTPSLDSQTGAIEVVPRKSIDTFGQRTSIYRGVTRHRWTGRYEAHLWDNSCRREGQTRKGRQVYLGGYDKEDKAARAYDLAALKYWGTTTTTNFPMSNYEKEIEEMKHMTRQEHVASLRRKSSGFSRGASIYRGVTRHHQHGRWQARIGRVAGNKDLYLGTFSTQEEAAEAYDIAAIKFRGLNAVTNFDMNRYDVNSIMESSTLPIGGAAKRLKEAEHAEITTRVQRTDDHDSTSSQLTDGISNYGTAAHHGWPTIAFQQAQAFTMHYPYGQRLWCKQEQDSDNHSFQELHQLQLGNTQNFLQPSVLHNVMSMESSSMEHSSGSDSVMYSSGGHDGTGTGTNGSYQGIGYGSNTGYAIPMATVIANDVNTQDQGNGYGDGEVKALGYENMFSSSDPYHARNLYYLSQQSSAGVIKASAYDQGSTCNNWLPTAVPTIAARSNNMAVCHGAPTFTVWNEST

SEQ ID NO: 42 - DNA, 1617 bp, Cajanus cajan
  source 1..1617: mol_type = other DNA
atggggtctatgaatttgttggggttttctctctctcctcaagaacacccttcgagtcaagatcactctcaaacggcaccttctcgttttggcttcaactctgatggaatctcaagcactgatgtagcaggagaatgctttgatctcacttctgactcaactcctcatttactcaatcttccttcttatggcatatacgaagcatttcacagaaacaataatattcacaccactcaagattggaaggagaactacaacagccaaaacttgctattgggaacttcatgcaacaaccaaaacatgaaccacaaccaacaacagcaacaacaacagccaaagcttgaaaacttcctaggtggacactcatttggtgatcatgagcaccaaacatacggtggtaactcagccacaacagattacattaactcgatagggttgtcaatgataaagacatggttgaggaaccaacccccacactcagaaaacaacaatgaaagtggtggcaatagcaaaagtaagagttcttcagataacaaacaaccactaaccacagttgcacttgataccacacaaactggagtcattgaaactgcacccagaaaatccatcgacacgtttggacaaagaacttctatataccgtggtgtaacaaggcataggtggacagggaggtatgaggctcacctgtgggataatagttgtagaagagagggacaaactcgcaaaggaaggcaagtttacttgggaggttatgacaaagaagaaaaggcagctagagcctacgatttggcagcactaaaatactggggaacaactacaacaacaaattttcctattagccactatgagaaagagttggaagagatgaagcacatgactagacaagagtacgttgcgtcgttgagaaggaagagtagtgggttttctcgcggtgcatccatttatcgaggagtaacgagacaccatcaacatggtagatggcaagcaaggatcggaagagttgcaggcaacaaggatctctacttgggaactttcagcactcaagaggaggcagcagaggcatatgatgttgcagccataaaattccgaggactgagtgcagttacaaactttgatatgagcagatacgacgttaagagcattcttgagagcaccactttgccaattggtggtgctgccaagcggttgcaccctatgatgagcatggattctgcttccattgacaatagctcttcatctaactcggttgtttatgatggttatggaggtggtggtggtgggggatatgtgattcctatgggaactactactactgttgttgcaagtgatggtgatcaaagtcaaagaagcaataatggttttggtgagaatgagatgaaggcacttggttatgaaagtgtttatggttctaatgatgcttatcatgcacatgcaaggaacttgtattatctttcccaacagcaaccatcttctgttgatgctgtgaaggctagtgcatatgatcaaggttcttcatgcaacacttgggttccaactgctattccaactcttgcaccaaggtctactagcatggctctttgccatggtgctccacccttctctttattgcatgaatag

SEQ ID NO: 43 - protein, 538 aa, Cajanus cajan
MGSMNLLGFSLSPQEHPSSQDHSQTAPSRFGFNSDGISSTDVAGECFDLTSDSTPHLLNLPSYGIYEAFHRNNNIHTTQDWKENYNSQNLLLGTSCNNQNMNHNQQQQQQQPKLENFLGGHSFGDHEHQTYGGNSATTDYINSIGLSMIKTWLRNQPPHSENNNESGGNSKSKSSSDNKQPLTTVALDTTQTGVIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLHPMMSMDSASIDNSSSSNSVVYDGYGGGGGGGYVIPMGTTTTVVASDGDQSQRSNNGFGENEMKALGYESVYGSNDAYHAHARNLYYLSQQQPSSVDAVKASAYDQGSSCNTWVPTAIPTLAPRSTSMALCHGAPPFSLLHE

SEQ ID NO: 44 - DNA, 2028 bp, Gossypium hirsutum
  source 1..2028: mol_type = other DNA
atggcttccatgaacaactggttagccttttctttatcaccacaagaacctgttgatcatcatcatcaatctcaaaccacagtttcccgtctcggtttcaactctgatggtatctccggtaccgatgtctcaggcgaatgcttcgatctctctggagactcatcggttccttctcttaatctacctccaccttttggcatacttgaagctttcaatagaaacagcaatcaaccccaagattggaatatgaaaggctcagagctttcaatggtgatgggtggtagttcatgcaatacccaagaaccaaagcttgaaaacttccttggaaaccatggctgtcacaccatgtacgatacaaccaccggtcaataccctctttacaccaccaccaccgcaaggacaaccaatggaggaggtgatgaggataacagtacaaacaacaacactaaccttactcacggagccagtaatagtaataccaatagcggcaactccataggtttatccatgatcaaaacttggttaagaaaccaacctgcaccaccaccgacacaagtcgagactaaaaacaacggtggtggaggcagcggcggaggtggtgcaccgcagtctttgtctctttcgatgagcactggtggcggcggcggtggaagcggtggagagagttcttcctctgacaacaagaaaacaccaccacaaccaccgtcaacaggtgtagaaatcacccaaaccggcaccaatgaagcggtggcaaggaaatctatcgacacttttggccaaagaacttccatataccgtggtgtaaccagacatagatggacaggtagatatgaagctcatttatgggataatagttgcagaagagaaggacaaagtcgtaaaggaagacaagtttatttgggagggtatgacaaagaagaaaaagcagctagggcttatgatttagcagctttgaagtattggggtactactacaacaactaattttccgattagcaactatgaaaaagagttggaagagatgaagcacatgacaaggcaggagtatgttgcttcactgcgaagaaagagtagtggattttctcgaggagcttccatgtatagaggtgttacaaggcaccatcaacatggaagatggcaagcaaggattggaagagtcgcaggaaataaagatctttacttgggaactttcagtacacaagaagaggcagcagaagcctatgacattgcagccataaaattccgaggactgaatgcggtaaccaacttcgatatgagtcgatatgacgtcaagagcatactagaaagcagtcagttaccgatcggtggcgccgccaaacgattgaaagacgttgagcaagcggaaatggctatggatatggtacataggtcggacggtgacaacattaatacgcatttacccgacggaatcaacacttacggtacatcaggatggcctaccattgcatatcaacaagctcaacctttcagcatgcattacccttacggacaacgaatttggtgcaaacaagagcaagattccgaccccgatcacgccttccacgatctccatcaactccaactaggaagcacccacaatttcttccaaccatccgtgttacacaacctaatggccatggaatcttcctcaatggaacatagctccggctctaataccaactcggtcatctacaacaacaccgtaagcgacggtgccgggtacggaggatacgctatccctttaggaacagtcattcctactgataccaaccaaaaccaaagcaacgatttcggagacaacagcaacaacgaagtgaaagcactcgggtacgaaaacgtgtacacatcatcggacccatatcattcgaggagcatgtactacctttcacaacaatcatcatcatcaacaagtcaagcctcaccatgcaacaattgggtgcccacagcagttcccaccattgcacaaaggtcaagcaatatggctgtatgccatggtaccccaacattcacagtatggaatgatacataa

SEQ ID NO: 45 - protein, 675 aa, Gossypium hirsutum
MASMNNWLAFSLSPQEPVDHHHQSQTTVSRLGFNSDGISGTDVSGECFDLSGDSSVPSLNLPPPFGILEAFNRNSNQPQDWNMKGSELSMVMGGSSCNTQEPKLENFLGNHGCHTMYDTTTGQYPLYTTTTARTTNGGGDEDNSTNNNTNLTHGASNSNTNSGNSIGLSMIKTWLRNQPAPPPTQVETKNNGGGGSGGGGAPQSLSLSMSTGGGGGGSGGESSSSDNKKTPPQPPSTGVEITQTGTNEAVARKSIDTFGQRTSIYRGVTRHRWTGRYEAHLWDNSCRREGQSRKGRQVYLGGYDKEEKAARAYDLAALKYWGTTTTTNFPISNYEKELEEMKHMTRQEYVASLRRKSSGFSRGASMYRGVTRHHQHGRWQARIGRVAGNKDLYLGTFSTQEEAAEAYDIAAIKFRGLNAVTNFDMSRYDVKSILESSQLPIGGAAKRLKDVEQAEMAMDMVHRSDGDNINTHLPDGINTYGTSGWPTIAYQQAQPFSMHYPYGQRIWCKQEQDSDPDHAFHDLHQLQLGSTHNFFQPSVLHNLMAMESSSMEHSSGSNTNSVIYNNTVSDGAGYGGYAIPLGTVIPTDTNQNQSNDFGDNSNNEVKALGYENVYTSSDPYHSRSMYYLSQQSSSSTSQASPCNNWVPTAVPTIAQRSSNMAVCHGTPTFTVWNDT

SEQ ID NO: 46 - DNA, 2106 bp, Manihot esculenta
  source 1..2106: mol_type = other DNA
atgaacaactggttgggcttctctctttccccgcaacaactcccatcacaatctgatcatcatcagaatcactctcaaaacacttcttctcgcttaggtttccactcagatgaaatctcagaaaccgatgtgtctggcgagtgcttcgatctcacttctaactcaactcttccttctctcaacctccccgccacttttggtattcttgaagctttcagaaacgatcaatctcaagattggaatatgaagggtttaggcatgaaccaggacactacctacaaaaccaactcagatcacctctccgtgctcatgggtacttcatgcagtagccaaaaccttgatcagaatcaagaacccaagttggaaaatttccttggtggccactccttcgctaatcatgagcacaagcttcctgcaggttgtaatactatgcacatgttccaaaactgctcttctcaacaggctacaccacatgatggaacaagcatttttggagaagataacaatagtatcaatactggtagtaatagtagctctattgggttgtctatgatcaagacttggctaaggaaccagcctgcaccgacacaacaacatgagcacaagaacaatggcggtggtgcacaaactttgtccctttctatgagtactgggtcacaatcaggctcgcctttgccacttctcggaacaaatactggcgaaaccaacaacaatggtggagataatcacagttcttctgataacaacaagcaaaagatagctactggtatcgacagccaaaccggcgcccttgagtcggttcccaggaaatctatagatacctttggccaaaggacatctatttatcgtggtgtaaccagacatagatggacaggtagatatgaggctcatctgtgggataatagttgcagaagagaaggacaaacccgaaagggaagacaagtttatttgggtggatatgataaagaagagaaggcagcaagagcatatgatttagcggcactaaaatactggggcaccactactacgacaaatttcccaattagcaactatgaaaaagagctagaagaaatgaagcacatgaccaggcaggagtatgttgcctctcttcgaaggaagagtagtggcttttctcggggtgcatccatctaccgaggtgttactagacaccatcagcatggaagatggcaagcaagaattggaagagttgccgggaacaaagacctttacttgggaactttcagtacccaagaggaagcagcagaagcatatgacattgcagccataaaattccgcggattgaatgctgtaaccaacttcgacatgagcagatatgatgtaaaaagcatcctggagagcaccactttacccattggaggagcagctaagcggttgaaagatgcagagcaggcagaaaggaaaatagattcacacagagcagaacatgaaaatatgagttctcagttttcagatggaatcagcagctatggcacagcacaccacccttggtctactgttgcatttcaacaatctcatcatcaaccttttagcttgcactatccttatgggcaaaggctttggtgcaagcaagaacaagatcaacttcaagtgggaaacacccacaatttctttcagccttctattttgcataacctcatgagcatggattcttcttcgatggaacatagctcagggtctaattcagttatttacagcagtggaggagcagatggtaacaatggagtcaatggaagctatcatggaatgggttcccgtgcaatcccaatggccactacagttgtggctaatgatgatggggaacaaaatcaagggaatggttatgaagatggagaagtgaaggttgttgggtatgaaaatatgttagggtcatcagaccctttccatggtagaagcttgtattatctttcacagcaatcagcagcagaaggaggaggtgtagtgaagggaataataagtgcatatgatgatgaggggtcagcgtgcaacaattgggttccaacagcagttcctacagttgggacaaggtcaagcaatatggctgtctgccatggagcttcaacttttgcagtgtggaaccacacatga

SEQ ID NO: 47 - protein, 701 aa, Manihot esculenta
MNNWLGFSLSPQQLPSQSDHHQNHSQNTSSRLGFHSDEISETDVSGECFDLTSNSTLPSLNLPATFGILEAFRNDQSQDWNMKGLGMNQDTTYKTNSDHLSVLMGTSCSSQNLDQNQEPKLENFLGGHSFANHEHKLPAGCNTMHMFQNCSSQQATPHDGTSIFGEDNNSINTGSNSSSIGLSMIKTWLRNQPAPTQQHEHKNNGGGAQTLSLSMSTGSQSGSPLPLLGTNTGETNNNGGDNHSSSDNNKQKIATGIDSQTGALESVPRKSIDTFGQRTSIYRGVTRHRWTGRYEAHLWDNSCRREGQTRKGRQVYLGGYDKEEKAARAYDLAALKYWGTTTTTNFPISNYEKELEEMKHMTRQEYVASLRRKSSGFSRGASIYRGVTRHHQHGRWQARIGRVAGNKDLYLGTFSTQEEAAEAYDIAAIKFRGLNAVTNFDMSRYDVKSILESTTLPIGGAAKRLKDAEQAERKIDSHRAEHENMSSQFSDGISSYGTAHHPWSTVAFQQSHHQPFSLHYPYGQRLWCKQEQDQLQVGNTHNFFQPSILHNLMSMDSSSMEHSSGSNSVIYSSGGADGNNGVNGSYHGMGSRAIPMATTVVANDDGEQNQGNGYEDGEVKVVGYENMLGSSDPFHGRSLYYLSQQSAAEGGGVVKGIISAYDDEGSACNNWVPTAVPTVGTRSSNMAVCHGASTFAVWNHT

SEQ ID NO: 48 - DNA, 2181 bp, Citrus sinensis
  source 1..2181: mol_type = other DNA
atgaataactggttaggtttctctctttctcctcaagaacaacttccttcctcacaaactgatcatcaccatcatcatcaagatcactctcaagactcagtctctcatcgccttggtttcaactccgatgaactctccggcgccgtcgtctccggtgactgctttgatctcacctctgctgactccactactgcctcctttagcatccttgaagccttcaacagaaacaacaatcactcccaagaatggaatatgaagggtttaggcatgaacacagacgctatgctaatggcgacttcatgcaatgatcaaaatcaagaaccaaagctcgaaaacttcctcgacggccactcgttctccaatcatgaacagaaactccatggctgcacctccgtttacgacacccccacggccagcaccgtcaccggagactacatgtttcaaaactgttctctacaacttcccttagatgagaacaacaataacaatgcaagaaccagcaacaacggaggcgaaaacagcaacaacaacaatagttccattgggttatcgatgatcaaaacttggctacgaaaccagcctgcaccagcaacgggaccggcaccagcaccggcccaagccgaggccgtgagcatgaatggcagtggtggcagtagtgcacagagtttatcactttctatgagcactggttcacagcctggctcaaccttgccgcttctcaatgcaaatggaggcttaaatggggtagaaagttcttgttctgataacaataagcagcaaaagacgacggcggggacagcaacggcaggaattgaccttgaaagccaaaccggggccattgaagcggtgccaaggaagtctattgacacttttggacagagaacttctatttaccgtggtgtcacaaggcataggtggactggtagatacgaggctcatttatgggacaatagttgcagaagagaaggacaaactcgaaagggaagacaagtttatctgggagggtatgacaaagaagaaaaggctgcaagagcttatgatttagcagcgttgaaatattggggtaccactaccactactaatttccctattagcaattacgagaaagaggtagaagaaatgaagcacatgaccaggcaagagtatgttgcttcactaagaaggaaaagtagtggattttctcggggtgcgtccatttaccgcggcgtaacaaggcatcatcagcatggtagatggcaagcaagaattggaagagttgcagggaacaaagacctttacttgggaactttcagcacacaggaagaagcagcagaggcctatgacatagcagcaattaaattccgaggactaaatgcggtaacaaattttgacatgaccagatatgatgtgaacagcattcttgagagcagcacattacccatcggaggagctgcaaagcgcctgaaagatgctgaacaggcagctgaaatgacaatcgatagaccgacggtggtggccgatgatgaaaacatgagctcacaactaacagctgatcatggctggcctgcaattgcagcattccaacaagcacaagctcatcatcatcatcagcatcagccatatagcagcttgcagccttttggatacggtcaaagaatttggtgcaagcaagaacaagatgagtttaatcatcagcttcaattgggaagcaacaacagcaatgcccacaatttctttcagccttcagtgatgcataatttgatgaacatggagtcatcttcattggaacacagctgtacttctaacaactctgtcatatacaacaacggagatcataccaatggcagctatcaagttggatatggaagcaacaacaacaacaacaatggtggatttgtgattccaatggggacagtaattgctagtaacaatgatcatgggtttggagaaagtgatcatcaagctgctgccaaagcacttgcttttgaaaacatgttttcatcatcaacatcttcttcagatccttatcaagctagaaacttgtattatcttcctcagcaacaatcatcagcaagtttggtgaaagctgctggtgcttatgataattgggtgcccactgctgtgccaactcttgctcaaaggtcaggcaatatagcactttgccatggagctccaactttcactgtgtggaatgatacataa

SEQ ID NO: 49 - protein, 726 aa, Citrus sinensis
MNNWLGFSLSPQEQLPSSQTDHHHHHQDHSQDSVSHRLGFNSDELSGAVVSGDCFDLTSADSTTASFSILEAFNRNNNHSQEWNMKGLGMNTDAMLMATSCNDQNQEPKLENFLDGHSFSNHEQKLHGCTSVYDTPTASTVTGDYMFQNCSLQLPLDENNNNNARTSNNGGENSNNNNSSIGLSMIKTWLRNQPAPATGPAPAPAQAEAVSMNGSGGSSAQSLSLSMSTGSQPGSTLPLLNANGGLNGVESSCSDNNKQQKTTAGTATAGIDLESQTGAIEAVPRKSIDTFGQRTSIYRGVTRHRWTGRYEAHLWDNSCRREGQTRKGRQVYLGGYDKEEKAARAYDLAALKYWGTTTTTNFPISNYEKEVEEMKHMTRQEYVASLRRKSSGFSRGASIYRGVTRHHQHGRWQARIGRVAGNKDLYLGTFSTQEEAAEAYDIAAIKFRGLNAVTNFDMTRYDVNSILESSTLPIGGAAKRLKDAEQAAEMTIDRPTVVADDENMSSQLTADHGWPAIAAFQQAQAHHHHQHQPYSSLQPFGYGQRIWCKQEQDEFNHQLQLGSNNSNAHNFFQPSVMHNLMNMESSSLEHSCTSNNSVIYNNGDHTNGSYQVGYGSNNNNNNGGFVIPMGTVIASNNDHGFGESDHQAAAKALAFENMFSSSTSSSDPYQARNLYYLPQQQSSASLVKAAGAYDNWVPTAVPTLAQRSGNIALCHGAPTFTVWNDT

SEQ ID NO: 50 - DNA, 2193 bp, Vitis vinifera
  source 1..2193: mol_type = other DNA
atggcttccatgaacaactggttgggtttctctttgtcccctcgagaacttccaccacagcctgaaaatcactcacagaacagtgtctctagacttggtttcaactctgatgaaatctctgggactgatgtgtcaggtgagtgttttgatctcacttcagattccactgctccctctctcaacctccctcccccttttgggatacttgaagcattcaacaggaataatcagccccaagattggaacatgaagggtttgggcatgaattcagatactaactacaaaaccaccacttctgagctctccatgctcatgggtagttcatgcagtagtcatcataacctcgaaaaccaagaacccaaacttgaaaatttcctgggctgccgctcttttgctgatcatgagcagaaacttcaagggtgtaactccattgcagcagcagcttatgatagctctgcagactacatgttccccaactgctcactgcagcttccatctgagccggtagacacccccactccccgcggtggcggcggtggaagcactaccgtcaacaatagttccattggtttatccatgatcaagacatggctgcggaaccaacctgcacccacccatcaggataacaacaagagtactgatactgggcctgtcggtggagccgccgctgggaacctacccaatgcacagaccttatcgttgtccatgagcaccggctcgcagtccagctctcctttgcctctcctaacagcgagtgcaggtggtggtggtgggagtggaggagagagttcttcatcagataacaagaaggccacccccctcgatagccagaccggtgccattgaaacggtgccaaggaagtccattgatacatttggacagaggacatccatataccgtggtgtaacaaggcatagatggacgggtagatatgaggctcatctatgggacaacagttgcagaagagaaggacaaactcgaaagggaaggcaaggtggttatgacaaagaagaaaaggcagctagggcttacgatttagcagcactgaagtattggggtaccaccaccacaacaaatttccctattagcaactatgaaaaagagatagaggagatgaagcacatgacaaggcaggagtacgtagcatctctgcgaaggaagagtagcgggttttctcgtggagcatccatatatagaggagtgaccagacaccatcagcatgggagatggcaggcaaggattggaagagtcgcaggcaacaaagatctttacttgggaactttcagcacccaagaggaagcagcagaggcctatgacattgctgccattaagtttcgaggattgaatgcggtgaccaactttgatatgagtagatatgatgttaatagcattctagagagcagtaccttgccgattggtggagctgcaaagcggttgaaagatgctgagcaggctgaaatgactatagatggacagaggacagacgatgagatgagctcacagctgactgatggaatcaacaactatggagcacaccaccatggctggcctactgttgcattccaacaagctcagccatttagcatgcactacccttatggccatcagcagagggctgtttggtgtaagcaagagcaagaccctgatggcacacacaactttcaagatcttcaccaactacaattgggaaacactcacaacttcttccagcctaatgttctgcacaacctcatgagcatggactcttcttcaatggaccatagctcaggctccaattcagtcatctatagcggtggtggagccgctgatggcagcgctgcaactggcggcagtggcagtgggagcttccaaggggtaggttatgggaacaacattggctttgtgatgcccataagcaccgtcatcgctcatgaaggcggccatggccagggaaatggtggctttggagatagcgaagtgaaggcgattggttacgacaacatgtttggatcgacagatccttaccatgctaggagcttgtactatctttcacagcaatcatctgcaggcatggtgaagggcagtagtgcatatgatcaggggtcagggtgtaacaactgggttccaactgcagttccaaccctagctccaaggactaacagcttggcagtatgccatggaacacctacattcacagtatggaatgatacataa

SEQ ID NO: 51 - protein, 730 aa, Vitis vinifera
MASMNNWLGFSLSPRELPPQPENHSQNSVSRLGFNSDEISGTDVSGECFDLTSDSTAPSLNLPPPFGILEAFNRNNQPQDWNMKGLGMNSDTNYKTTTSELSMLMGSSCSSHHNLENQEPKLENFLGCRSFADHEQKLQGCNSIAAAAYDSSADYMFPNCSLQLPSEPVDTPTPRGGGGGSTTVNNSSIGLSMIKTWLRNQPAPTHQDNNKSTDTGPVGGAAAGNLPNAQTLSLSMSTGSQSSSPLPLLTASAGGGGGSGGESSSSDNKKATPLDSQTGAIETVPRKSIDTFGQRTSIYRGVTRHRWTGRYEAHLWDNSCRREGQTRKGRQGGYDKEEKAARAYDLAALKYWGTTTTTNFPISNYEKEIEEMKHMTRQEYVASLRRKSSGFSRGASIYRGVTRHHQHGRWQARIGRVAGNKDLYLGTFSTQEEAAEAYDIAAIKFRGLNAVTNFDMSRYDVNSILESSTLPIGGAAKRLKDAEQAEMTIDGQRTDDEMSSQLTDGINNYGAHHHGWPTVAFQQAQPFSMHYPYGHQQRAVWCKQEQDPDGTHNFQDLHQLQLGNTHNFFQPNVLHNLMSMDSSSMDHSSGSNSVIYSGGGAADGSAATGGSGSGSFQGVGYGNNIGFVMPISTVIAHEGGHGQGNGGFGDSEVKAIGYDNMFGSTDPYHARSLYYLSQQSSAGMVKGSSAYDQGSGCNNWVPTAVPTLAPRTNSLAVCHGTPTFTVWNDT

SEQ ID NO: 52 - DNA, 2469 bp, Malus domestica
  source 1..2469: mol_type = other DNA
atgggttccatgaactggttgggtttctctctttcccctcaagaagtagttccggctgatccaacgatatctgagggtactgatcatcgtcatcagcagcacggccaagatcatcaggacttatctgaccagacactagtctctcgcctccgctctaagtactcggatgaaatctccggtaccgatgcctccggagactgctttgatctcactaattctcatgacgactcatctgctactcctactgctcctaatcatcattctctcaaccacctccctacatcttttgggatgcttcatcaagctgcgttcgactcccaagcaggtgcagattggaatatgaactcaccggatagcaccaactacaagttcacatcagaccacctctctatgcttaactcatgcaataccctaaaccttgaaaaccatcaaaatcgtcatcagcagcagccaaagctcgaaaacttcctgggccgacactctttcattgcggatcatccagatcatatccagagtagcactagttctgcgcatgcatacaatagtaaaaacaccgccagtattaatactaccgacaacatcttcaccagaaccgtctcttctttgcagcttccgttagagatttccgatcccacaaatgtcatcaacggagacggtgcagtcggtaacattaatatcaacagcagcacctcgaatattgggctttcaatgatcaagacatggctgaggaatcaaccggcatcccagctgcaagagatgaacaagaacgatgtcaatggcatcggtgtctccaacacaagtactagtactagcagtgcacagactctttcgctttcaatgagtactgcaggtgctactacggccggagatcaaacctgttcgtcagatcgagatcgagatagtaataagcaggcaattattaggaccgctgcccccgcagcaactattcagtctgggatcgataaccagactagtattgctactgctattgaggccgtgcccagaaagtccattgatacatttggacagagaacttctatataccgtggtgtaaccaggcacagatggacaggtagatatgaagctcatttgtgggataatagttgccgaagagagggacaaactcgaaagggacggcaagtttatctgggtggttatgacaaggaagaaaaggccgcaagagcttatgacctagcagcattgaaatattggggtactacaaccactaccaacttcccgattagcaactatgagaaagaggtagacgaaatgaagcacatgacaagacaggaatatgttgcatctttgcgaaggaagagtagtgggttttctcgtggtgcatccatttatcgaggagtgacaaggcaccaccagcatgggagatggcaagcaaggattggaagagtagcagggaacaaagatctctacctgggaacattcagtacccaggaggaagcagcagaggcatatgacatcgcagccataaaattccggggacttaatgcagtaactaactttgacatgactagatacgacgtgaagtccattctggagagcagcacattgcccattggtggtgctgcgaagcgactgaaagacgttgagcaggcacatcaccagcatctgcacgagatgactctactggccgacggacacaacagatcaacgatggatgatcaacgcgaaaagctgatgatgatgaatactagtagtaatagtctcgatcaatatcatcagcaactgataaatggagatcatcatggaatgagaataaataacgtctatggtactggagcaggctggccaactcttgcatttaaccaagccgctaccgctgttgcccatcaggccgcagtgccgccctttgggatgcattattactcatcagtgccatataattctaatggcgggcagagggtttggtgcaagcaagagcaagacactaattctcaaagcttttatcagccacaagatcatcatcgtcaagatcttcatcatcatcaactacttcagttgggaagtactcacaatttctttcagccagtcaacctcatgggaaatagtactacggattctatggaacatagctcaggttctaattctgtcatatataatggtactactgccggagatcacggtggctatataatccctatggggacggctatagctaatgatcatgagaataatagtaatgcttttggaaatggcagttccgatcatgatcaggtgaagccggcgctaggttatgaaaatgcgtttggctcaagtagtactactaccactgtggatgcttataatcatgcaaagagcttgtattatcttccggttcaggcgccacagtcatcatcagtttcttcaataggtgtgtcgacgaagggaagcagcgagtatgacaattgggtgccaacagcagttccaacatctactcacaatatggctcctttcacagtctggaatgacacataa

SEQ ID NO: 53 - protein, 822 aa, Malus domestica
MGSMNWLGFSLSPQEVVPADPTISEGTDHRHQQHGQDHQDLSDQTLVSRLRSKYSDEISGTDASGDCFDLTNSHDDSSATPTAPNHHSLNHLPTSFGMLHQAAFDSQAGADWNMNSPDSTNYKFTSDHLSMLNSCNTLNLENHQNRHQQQPKLENFLGRHSFIADHPDHIQSSTSSAHAYNSKNTASINTTDNIFTRTVSSLQLPLEISDPTNVINGDGAVGNININSSTSNIGLSMIKTWLRNQPASQLQEMNKNDVNGIGVSNTSTSTSSAQTLSLSMSTAGATTAGDQTCSSDRDRDSNKQAIIRTAAPAATIQSGIDNQTSIATAIEAVPRKSIDTFGQRTSIYRGVTRHRWTGRYEAHLWDNSCRREGQTRKGRQVYLGGYDKEEKAARAYDLAALKYWGTTTTTNFPISNYEKEVDEMKHMTRQEYVASLRRKSSGFSRGASIYRGVTRHHQHGRWQARIGRVAGNKDLYLGTFSTQEEAAEAYDIAAIKFRGLNAVTNFDMTRYDVKSILESSTLPIGGAAKRLKDVEQAHHQHLHEMTLLADGHNRSTMDDQREKLMMMNTSSNSLDQYHQQLINGDHHGMRINNVYGTGAGWPTLAFNQAATAVAHQAAVPPFGMHYYSSVPYNSNGGQRVWCKQEQDTNSQSFYQPQDHHRQDLHHHQLLQLGSTHNFFQPVNLMGNSTTDSMEHSSGSNSVIYNGTTAGDHGGYIIPMGTAIANDHENNSNAFGNGSSDHDQVKPALGYENAFGSSSTTTTVDAYNHAKSLYYLPVQAPQSSSVSSIGVSTKGSSEYDNWVPTAVPTSTHNMAPFTVWNDT

SEQ ID NO: 54 - DNA, 2034 bp, Solanum lycopersicum
  source 1..2034: mol_type = other DNA
atggcttcttcaatgaacaattggcttggattttcactttcaccacatgaagaagtactcacatcacaacaaagttcacatcaagaatgttttgatctattgacttctcatcatcatcaacaacatcatgatcaatcaacagtagtacttccttctttaaatgttgtacctcatgataataccccttttggtatttttgaagctttcaatagaaatcatcatcaatcacaagattggaactctcatgatacaaactacaagactacatcaaatatgtcaatgttaatgggaacttcatgcaatagtcaacatcttgaaaataatcaagaaccaccaaagctagagaactttcttggtattggtgaacaaaagcttcaacaagtctgcaataacaataacaataacaataacaacaacaactattacaatatctattgtagtacccctaataatattagcgaaaataacgctaataatattatagggctctcaatgatcaagaattggttgaggaacaaccctaactccactaccaccaccactactaccccgtcatcattacaagatgataagaaggagggtgatgttgtgggggtagccggtggtggtggtaatggtaataatgcacgatcgttgtcactttctatgagcatgggtggtgatggaggtggagataatagttgttcgtccgaaaataacaaacaagagattgtgagtggaaataatgatggacaaataagtggtgcaattgtagtaccaaagaaatctattgacacttttggtcaaagaacttcaatctatcgcggtgttacaaggcataggtggactgggagatatgaagcacatttgtgggataatagttgtagaagagagggacaaactagaaaaggaaggcaagtttatttgggaggatatgataaggaggaaaaagcagctcgtgcttatgatttagctgcacttaagtattggggtcccacaactaccactaatttcccaattagcaactatgagaaagagcttgaagaaatgaagcatatgactaggcaggaatatgttgcatctttaagaaggaagagcagtggtttctctcgtggtgcatcgatttatcgaggtgttacaaggcatcatcagcatggaagatggcaagcaagaattggtagagttgctggaaacaaggatctttatcttggcacctttagtacacaagaagaagctgctgaggcttatgacattgcagcaatcaaattccgtggactaaatgccgtaacaaattttgagattaatcgatacgacgttaaaagtatactagaaagcagcacgttgcccatcggaggggctgctaaacgtctcaaggacgttgagcaagctgaaatcgcgttggattatcagagagcaagtcaagaaaatattggtactactcatcttatgaatggttcaatgagtgcctatggcgcgggccattgttggcccaacatcactcttcaacaagctcaacctttaagcgcgatgcattatagtaacccttataatagtcaacaaaggttgtggtgcaaacaggaagtacaagatcatgatttgactagtcaaaattttcaagactttcaattgggaaacgcgcataattatctgatgggattggataattcttcttcatcgtcaatggaacaaaataacgttacgtatcatcaaggtgttggatatgcgagtaacaataataatcatcatggtggtggtggatttgtgttgcctcatttgaacggatcatcatcatatggtggagaaaatgataatgaagtgaagcaattacaacttggtactcatgagaacatatttggtaattactaccattcgcaaccagcaacatctaacgactcaatcaaggtcagtaacactctatatgatcatcaagaatctgtatgcaacagtaataactggatgtcaacagctgttcctatggctcttgcttcgaggactagcaccgcggcgatttgccatggtgtggcccccactttcactatgtggaatgactcatag

SEQ ID NO: 55 - protein, 677 aa, Solanum lycopersicum
MASSMNNWLGFSLSPHEEVLTSQQSSHQECFDLLTSHHHQQHHDQSTVVLPSLNVVPHDNTPFGIFEAFNRNHHQSQDWNSHDTNYKTTSNMSMLMGTSCNSQHLENNQEPPKLENFLGIGEQKLQQVCNNNNNNNNNNNYYNIYCSTPNNISENNANNIIGLSMIKNWLRNNPNSTTTTTTTPSSLQDDKKEGDVVGVAGGGGNGNNARSLSLSMSMGGDGGGDNSCSSENNKQEIVSGNNDGQISGAIVVPKKSIDTFGQRTSIYRGVTRHRWTGRYEAHLWDNSCRREGQTRKGRQVYLGGYDKEEKAARAYDLAALKYWGPTTTTNFPISNYEKELEEMKHMTRQEYVASLRRKSSGFSRGASIYRGVTRHHQHGRWQARIGRVAGNKDLYLGTFSTQEEAAEAYDIAAIKFRGLNAVTNFEINRYDVKSILESSTLPIGGAAKRLKDVEQAEIALDYQRASQENIGTTHLMNGSMSAYGAGHCWPNITLQQAQPLSAMHYSNPYNSQQRLWCKQEVQDHDLTSQNFQDFQLGNAHNYLMGLDNSSSSSMEQNNVTYHQGVGYASNNNNHHGGGGFVLPHLNGSSSYGGENDNEVKQLQLGTHENIFGNYYHSQPATSNDSIKVSNTLYDHQESVCNSNNWMSTAVPMALASRTSTAAICHGVAPTFTMWNDS

SEQ ID NO: 56 - DNA, 942 bp, Arabidopsis thaliana
  source 1..942: mol_type = other DNA
atgatggttgatgaaaatgtggaaaccaaggcctctactttagtggcaagtgttgatcatgggtttggttccgggtcgggtcatgatcatcatgggttatcggcgtctgtgcctcttcttggtgttaactggaagaagagaaggatgcctagacagagacgatcttcttcttcctttaaccttctctctttccctcctcctatgcctcctatttcccacgtgccaactcctctccccgcacgtaaaattgacccaagaaagctaagattcctcttccaaaaggaactcaagaacagtgacgtcagctctctccgacgtatgatactcccgaagaaagccgcggaggctcacttgccggcacttgaatgcaaggaagggattcctataagaatggaagatttggacggttttcacgtttggaccttcaagtataggtactggccaaacaacaatagcagaatgtacgtgctagaaaacacaggcgattttgtgaatgctcatggtctgcagctaggtgacttcatcatggtttaccaagatctctactcaaacaattacgttatacaagcaagaaaagcatcggaagaagaagaagtagacgtaatcaatcttgaagaagacgacgtttacacaaacttaacaaggatcgaaaacactgtggttaacgatcttctcctccaagattttaatcatcacaacaacaacaacaacaacaacagcaacagcaacagcaacaaatgttcttactattatccagtcatagatgatgtcaccacaaacacagagtcttttgtctacgacacgacggctcttacctccaacgatactcctctcgattttttgggtggacatacgacgactactaataattattactccaagttcggaacattcgatggtttgggctccgttgagaatatctctctcgatgacttctactag

SEQ ID NO: 57 - protein, 313 aa, Arabidopsis thaliana
MMVDENVETKASTLVASVDHGFGSGSGHDHHGLSASVPLLGVNWKKRRMPRQRRSSSSFNLLSFPPPMPPISHVPTPLPARKIDPRKLRFLFQKELKNSDVSSLRRMILPKKAAEAHLPALECKEGIPIRMEDLDGFHVWTFKYRYWPNNNSRMYVLENTGDFVNAHGLQLGDFIMVYQDLYSNNYVIQARKASEEEEVDVINLEEDDVYTNLTRIENTVVNDLLLQDFNHHNNNNNNNSNSNSNKCSYYYPVIDDVTTNTESFVYDTTALTSNDTPLDFLGGHTTTTNNYYSKFGTFDGLGSVENISLDDFY

SEQ ID NO: 58 - DNA, 21335 bp, synthetic construct
  misc_feature 1..21335: note = Plasmid
  source 1..21335: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaatggtggaggcggctcgggtggcggaggctccggaggcggtggctcgacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaagtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 59 - DNA, 21359 bp, synthetic construct
  misc_feature 1..21359: note = Plasmid
  source 1..21359: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattaggtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccaaccatggatattcgaattggccaatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggggtggaggcggctcgggtggcggaggctccggaggcggtggctcgacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 60 - DNA, 21698 bp, synthetic construct
  misc_feature 1..21698: note = Plasmid
  source 1..21698: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaatggtggaggcggctcgggtggcggaggctccggaggcggtggctcgacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaagtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccaaccatggatattcgaattggccaatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggggtggaggcggctcgggtggcggaggctccggaggcggtggctcgacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 61 - DNA, 20104 bp, synthetic construct
  misc_feature 1..20104: note = Plasmid
  source 1..20104: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttggtggaggcggctcgggtggcggaggctccggaggcggtggctcgacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaagtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 62 - DNA, 19766 bp, synthetic construct
  misc_feature 1..19766: note = Plasmid
  source 1..19766: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcggtacggccagaatggcccggaccgggttacccggaccggaattcgagctccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgtttaggtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgccccgggttaacggcaaacgctaattagtcactaggcccaggaaacactaactaagccctagcttatcctagcgcaacctagttaggtcttccccaagtttgtacaaaaaagcaggctggatcggccagaatggccccggaccgggttaccaagcggccgcttaaaaccttattaagtggcatgggtaacggttcaatatgatcctatcggatcaaaccgggttacatatattattgaacccggttttattggtacgatcgttaagtctcaacatctggctctaataaccgggacgatagagattgtggttagaaataaacggcgttaaaattggttgcagccgcgggcacacggatcgtgtttatcaaataaggcaattagccaaaaaatcgtttcgtgtaaaagttacgtgtcaagctttggttggttattgcttcaccgccttagctttctcgtgaccgactcgtcctcgtctttttttcttcttctataaattaataacctaagacctattcttcttcacaaattcagattccaaatttctcaaactctgaaatcgttctttcaaatctctcaatcgggatcaaggtaaatttctgggttcttattcagtttcttcgattattttccattcgatcgcaatttcgtccctgttctttgagtttgattcggttaatctttgatcggagataaatatctgagtttcatcgctagatctcatctatattctgaactagggttcgtagatttcacagatttgtatatttaggcgtcgattctttgaatttgatccgatatgtgatttatattcagatctggtggtagcttttttaatcggatttgagattatccaagcgattaatctcagttttttgtgaattacagggatccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccaaacaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcatgggcgcgccggtcacccggtccgggcctagaaggccacccggggtcggaggagactagccgttagttagtgcagaccgtctctaatcagtgactagcgacgtcccgacccacccagctttcttgtacaaagtggcccggggcggccgaagctggccttctaggcccggaccgggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatcatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaagctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 63 - DNA, 2163 bp, Arabidopsis thaliana
  source 1..2163: mol_type = other DNA
atgaaaagcttgcatgtggcggccaacgccggagatctggctgaggattgtggaatactcggtggagacgctgatgatactgttttgatggatggaattgatgaagttggtagagagatctggttagatgaccatggaggagataataatcatgttcatggtcatcaagatgatgatttgattgttcatcatgacccttcaatcttctatggagatctcccaacgcttcctgatttcccatgcatgtcgtcttcatcatcgtcttcaacatctccagctcctgtcaacgcaatcgtctcctcagcctcttcttcttcggcagcttcttcctccacttcctcagctgcttcttgggctatattgagatcagatggagaagatccgactccaaaccaaaaccaatacgcatcaggaaactgtgacgactcttctggtgcattgcaatccacagcttccatggagattccattagacagcagtcaaggttttggttgcggcgaaggcggtggtgattgcattgatatgatggagactttcgggtacatggatctacttgatagcaacgagttctttgacacctcagctatatttagccaagacgacgacacgcaaaaccctaacttgatggaccaaacccttgagagacaagaagaccaggtcgttgttccgatgatggagaataacagtggtggagacatgcaaatgatgaattcttccttggaacaggacgatgatctcgctgctgtgtttttggagtggctaaagaacaacaaggagactgtgtcggctgaggatttgaggaaagtaaagataaagaaagctacgattgaatcagcggcaagaagactaggcggtggtaaagaagcgatgaagcagcttttaaagctgattcttgaatgggtccaaactaatcacttacaaagaagacgcaccaccaccaccaccaccaacctctcttatcaacaatcattccaacaagatccatttcaaaaccctaaccctaataacaacaacctaatcccaccgtccgaccaaacctgtttctcaccttcaacatgggttcctccaccaccacaacaacaagcttttgtctcggacccgggttttggatacatgcctgctccaaactatccgccacagccagagttccttcctttacttgaatctccaccgtcatggccaccaccaccacagtctggtcccatgccacatcaacaattccccatgccgccaacctcgcagtataatcaatttggagatccaacaggtttcaatggatacaacatgaatccgtaccaatatccttatgttcctgcaggacaaatgagagatcagagattactccgtttgtgttcctcagcaactaaagaggcaagaaagaaacggatggcgagacagaggaggttcttgtctcatcaccacagacataacaacaacaacaacaacaacaacaataatcagcagaaccaaacccaaatcggagaaacctgtgccgcggtggctccacaacttaaccccgtggccacaaccgccacgggagggacctggatgtattggcctaatgtcccggcagtgccgcctcaattaccgccagtgatggagactcagttacctaccatggaccgagctggctcagcttctgctatgccacgtcagcaggtggttccagatcgccggcagggatggaaaccagaaaagaatttgcggtttctcttgcagaaagtcttgaagcaaagcgacgtgggtaacctcggaaggatcgttttgccaaaaaaagaagctgagacacacttgccggagctagaggcaagagacggcatctctctggccatggaagacatcggaacctctcgtgtttggaacatgcgctacaggttttggcctaacaacaaaagcaggatgtatctcctcgagaacaccggcgattttgtgaaaaccaatgggctccaagaaggtgatttcatagtcatatactccgacgtcaaatgtggcaaatatttgatacgaggggttaaagtaagacaaccgagcggacaaaagccggaggccccaccgtcgtcagcagctacgaagagacaaaacaagtcgcaaaggaacataaacaataactctccgtcggcgaatgtggtggtcgcttcaccaacttctcaaactgttaaatga

SEQ ID NO: 64 - protein, 720 aa, Arabidopsis thaliana
MKSLHVAANAGDLAEDCGILGGDADDTVLMDGIDEVGREIWLDDHGGDNNHVHGHQDDDLIVHHDPSIFYGDLPTLPDFPCMSSSSSSSTSPAPVNAIVSSASSSSAASSSTSSAASWAILRSDGEDPTPNQNQYASGNCDDSSGALQSTASMEIPLDSSQGFGCGEGGGDCIDMMETFGYMDLLDSNEFFDTSAIFSQDDDTQNPNLMDQTLERQEDQVVVPMMENNSGGDMQMMNSSLEQDDDLAAVFLEWLKNNKETVSAEDLRKVKIKKATIESAARRLGGGKEAMKQLLKLILEWVQTNHLQRRRTTTTTTNLSYQQSFQQDPFQNPNPNNNNLIPPSDQTCFSPSTWVPPPPQQQAFVSDPGFGYMPAPNYPPQPEFLPLLESPPSWPPPPQSGPMPHQQFPMPPTSQYNQFGDPTGFNGYNMNPYQYPYVPAGQMRDQRLLRLCSSATKEARKKRMARQRRFLSHHHRHNNNNNNNNNNQQNQTQIGETCAAVAPQLNPVATTATGGTWMYWPNVPAVPPQLPPVMETQLPTMDRAGSASAMPRQQVVPDRRQGWKPEKNLRFLLQKVLKQSDVGNLGRIVLPKKEAETHLPELEARDGISLAMEDIGTSRVWNMRYRFWPNNKSRMYLLENTGDFVKTNGLQEGDFIVIYSDVKCGKYLIRGVKVRQPSGQKPEAPPSSAATKRQNKSQRNINNNSPSANVVVASPTSQTVK

SEQ ID NO: 65 - DNA, 22174 bp, synthetic construct
  misc_feature 1..22174: note = Plasmid
  source 1..22174: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcgccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattaggtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaccggtagattagtgggccaagcgtttaatcagggttaagagtggctactaacggctaggtcgctagctatttcctcgtgagaccttcttgtaactccggacaagtttgtacaaaaaagcaggctggatcggccagaatggccccggaccgggttaccaagcggccgccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggatccaaccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaggcgcgcccgggattaagacccgtcgtcaagtaggaattccagaaatcaacgcacggtaactaaatcctcgcgctaagctgggtcagcgctacccagctttcttgtacaaagtggggatcggccagaatggcccggaccgggttaccaagcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggtgtccaagggggaggagctcattaaagagaacatgcacatgaagttatacatggagggcaccgtgaataatcaccacttcaagtgtacctctgagggtgagggcaagccctatgagggaacccagactatgagaatcaaggtggtcgaaggaggtccactcccattcgccttcgacatactagccacctccttcatgtacgggagtagaaccttcatcaaccacacccagggcatcccagacttcttcaaacagtcattcccagagggattcacctgggagagagtgaccacctacgaagacggaggagtcttgaccgccacccaagacactagtcttcaagacggctgcttgatctacaacgtcaagatccgcggcgtgaacttcccatcaaacggtcccgtgatgcagaagaagactctcggctgggaagccaatactgaaatgctttacccagccgacggtggtcttgaaggtagatccgacatggccttgaagcttgtcggcggaggccatctcatctgtaacttcaagaccacctaccgctccaaaaagcctgccaaaaacttgaaaatgcccggcgtgtattacgtggaccatagactcgagcgcataaaagaggccgacaaggagacttatgtggagcagcacgaggtggccgtcgctagatattgcgacttgccatcaaagcttggccacaaactgaattaggcgatcgctagatctttgagctcttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggttaaccccggggtcggaggagactagccgttagttagtgcagaccgtctctaatcagtgactagcgacgtcccgacccacccagctttcttgtacattcaaagccaatgactgttgttaggtccagtttctaaggacgaatcatccaatcactggttctttctgaacaaaacaaagagctatagagttacgttactgtagccgagacagagcctgcaatctggttatgccacatactaggttctttcttaaagttgactcactctatcctaagttctacttacaaggagctacaacaaattggtcataaccggagagcgttcaaggaggtaatatcccatcttactctgtagcaaggctacaaaaggagagctaaagaactaggctacaaaaagagagctaaagaactaactatctagcaagactacagagctatagtgtagagatttcataaccggtgagcgttcaaggatactttgaagagaagaggtaatatctcaactagcaagactacaaaaaaatggagatataaaactaactgtctaacaagactacagagctatagtagagatagatacgttcgacacttaagcatgaggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagcggccgcaattggatcctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 66 - DNA, 21811 bp, synthetic construct
  misc_feature 1..21811: note = Plasmid
  source 1..21811: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcgccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattaggtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaccggtagattagtgggccaagcgtttaatcagggttaagagtggctactaacggctaggtcgctagctatttcctcgtgagaccttcttgtaactccggacaagtttgtacaaaaaagcaggctggatcggccagaatggccccggaccgggttaccaagcggccgcccagcatttgccacgttcgaacgtgagccaaaacgatgtcgttacaatatcttaacctagccgaaacgatgtcgtggtaactcataatatcgcagccttctgtttcgtccttgcccaatgccaactggactacgccgaacccacaaaacccgacatccgcgcgaaccaaagcgcgtgacgccaaattgctcaaaccacaaaggaaacaaaggcgcgttaataacacgcgctatcccacgcgtgttaagcacagtaacgttcaacaaagagaagcagggtataatgggaaaccaaagagaaacctaaggccattatgggaaacaaaaagtagctcactcattataaaattcaagtaaccctagttttcatcatcactctgctgcctcgctctgtttctgtctctgtgtttgcggctgaggattccgaacgagcgaccttcttcgtttctcgcaaaggtaacagcctctgctcttgtctcttccattcgatccatgcctgtctcttctttacgatgatgtttcttcggtgtatgtttatttatttatttatttatgctttatgttgtgaatgttcggttgttttgtttcgctttgcttttgtggattctattggtttttgaatcagttaatcggaagagattttcgagttgttttgtgttttggaggtgaatctttttgttgaggtcgcagatctgttgattttgtgtcataaacgtgcgactctgtttgattttttacgaggttatgacgttttggttgttttattatggatctgttaaggcagaaccatgatttatgtttatgttcgtttacacgattaaattttcttgtaacacgatgaagtttttttaaacacgttgaaggagtcttgttgatatgaatttgtcgattgtttttttgtggttttgttctcacgttatcaagcgtaatcttttactatgtacgcgaacatatctagatctagcagagctttttttttttttaattctttgtgaagcttttgaaatatgaaatttgtttttcaaatttttttttaatttattgaaaacactgtggatactgaataaggttctatgtatgatgcgaaccatgtttgatatgtttgtttttgttgttccatatggatttttctgttagatttccatatgcttttgagtttgtttttgctgttacagattgatttactttttagaatatttctcttggctttcactgttttagaaattttttgttattggtaactataaatgtgtgaatttggattatacctttaccttgttatttagttgtgtgataatttggtttatagttattttgagttctgactcgtgtttctttgaattgattccagtttaagtcatcggatccaaccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaggcgcgcccgggattaagacccgtcgtcaagtaggaattccagaaatcaacgcacggtaactaaatcctcgcgctaagctgggtcagcgctacccagctttcttgtacaaagtggggatcggccagaatggcccggaccgggttaccaagcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggtgtccaagggggaggagctcattaaagagaacatgcacatgaagttatacatggagggcaccgtgaataatcaccacttcaagtgtacctctgagggtgagggcaagccctatgagggaacccagactatgagaatcaaggtggtcgaaggaggtccactcccattcgccttcgacatactagccacctccttcatgtacgggagtagaaccttcatcaaccacacccagggcatcccagacttcttcaaacagtcattcccagagggattcacctgggagagagtgaccacctacgaagacggaggagtcttgaccgccacccaagacactagtcttcaagacggctgcttgatctacaacgtcaagatccgcggcgtgaacttcccatcaaacggtcccgtgatgcagaagaagactctcggctgggaagccaatactgaaatgctttacccagccgacggtggtcttgaaggtagatccgacatggccttgaagcttgtcggcggaggccatctcatctgtaacttcaagaccacctaccgctccaaaaagcctgccaaaaacttgaaaatgcccggcgtgtattacgtggaccatagactcgagcgcataaaagaggccgacaaggagacttatgtggagcagcacgaggtggccgtcgctagatattgcgacttgccatcaaagcttggccacaaactgaattaggcgatcgctagatctttgagctcttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggttaaccccggggtcggaggagactagccgttagttagtgcagaccgtctctaatcagtgactagcgacgtcccgacccacccagctttcttgtacattcaaagccaatgactgttgttaggtccagtttctaaggacgaatcatccaatcactggttctttctgaacaaaacaaagagctatagagttacgttactgtagccgagacagagcctgcaatctggttatgccacatactaggttctttcttaaagttgactcactctatcctaagttctacttacaaggagctacaacaaattggtcataaccggagagcgttcaaggaggtaatatcccatcttactctgtagcaaggctacaaaaggagagctaaagaactaggctacaaaaagagagctaaagaactaactatctagcaagactacagagctatagtgtagagatttcataaccggtgagcgttcaaggatactttgaagagaagaggtaatatctcaactagcaagactacaaaaaaatggagatataaaactaactgtctaacaagactacagagctatagtagagatagatacgttcgacacttaagcatgaggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagcggccgcaattggatcctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 67 - DNA, 21867 bp, synthetic construct
  misc_feature 1..21867: note = Plasmid
  source 1..21867: mol_type = other DNA
aattacaacggtatatatcctgtcagccaacaccaaaacatcaaaaaaatcgggatcaggatcaggatctgcggccgcatcctgcaggtttaaacggccggccttcactagctagctgctaagctcaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaatcgagctagttaccctatgaggtgacatgaagcgctcacggttactatgacggttagcttcacgactgttggtggcagtagcgtacgacttagctatagttccggacttacctaactgactagagtcacacttagctgaccctagtcacttaataacttcgtatagcatacattatacgaagttatggcgcgccaagcttggatctcctgcaggatctggccggccggatgggcggatctcgtacctcaactttgtatagaaaagttgggccgaattcgagctcgccaccgcggtggcggccgctctagaaagcttagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagcggccgggatccatgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattaggtttaaacccgggttaacaggcctgaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaccggtagattagtgggccaagcgtttaatcagggttaagagtggctactaacggctaggtcgctagctatttcctcgtgagaccttcttgtaactccggacaagtttgtacaaaaaagcaggctggatcggccagaatggccccggaccgggttaccaagcggccgctgtggtcataatccaaggactcaatttaattctttttttatatttcattaatgttgtaaggttaatttggaaaataaaacaaagggaagacattcaacttcccctctctctagctgcagttaaaccacatgtgagccttaagatgtacacgtgtcaagctataagtaagacatttctttataatgaaaaatacaaaaataaaaaaaaaataaaaattctaagcaatattgagtttctttgattaagatctctggatggatcagatagcaaacttggttttgactggtttggcaaaatgaagaaaaacaacgaaagaaagatgaatttatgtttattttaaatataaagtttttggacgcaaaagtgaaaattttaaacattcttatatacagatatataaataccaaactgaattgatgaagtaatctctttttttttttgggttattctgaagcttaacactgaagaaaactggtgaagttgagaatttcctttaattgcagtatggagcatgtttgtgttcctagagccacaagcaacacgtagaagagacaaggagagagccagaggaagaggcagagagagtaaaagaacaaggagcttgttatcatcgtagggtccatgaatgtaattaataggactcaatcaccttcatcaaattataatccgactcaccaaaattaatttcttgtatctacccaataataattgagaaacaaatcttatattaataatataaaataattttacatattattgaatcataaattatcttaaaattaaataatgttataaaattattttacgttctaagtggcattaaactcataataattcatgttaaatttatcttcatctgaaaatactctaagattctaagttaacataatttcatgaaaattgaaaattggatatgttggctactatgtggggtgaacagtaccatctttacgtaaataaaacaccacctcaaatattatggtagttgttctttaaaaaatactgataatattatcattggttatcaagattgaagaggactgaaatgacaataaccaatgctttgaatttgaataaacgacaaaagttctcctcacaggcttgtgtgtgtggcaataacatctccttaatactggtggagatagaaaaagcttgaataccaaccgcaaagagtaggactaagagtgacaaaaaaccttgcaggcacattatgttgccacgtgtcggctgatcaatcttctatgtgtggatgtaaggcttctatgaccaatccttttgtgtacactcactccaccaagcaactactataagtcacagcctcattcatggtcacaagccacttagcactgagtcttgcaattgaagttgaagtcttgcccttctctcttcaactccaaacacaagacatcatcatccaggatccaaccatggataacttcttaccctttccctcttctaacgcaaactctgtccaagaactctctatggatcctaacaacaatcgctcgcacttcacaacagtccctacttatgatcatcatcaggctcagcctcatcacttcttgcctccgttttcatacccggtggagcagatggcggcggtgatgaatcctcagccggtttacttatcggagtgttatcctcagatcccggttacgcaaaccggaagtgaattcggttctctggttggtaatccttgtttgtggcaagagagaggtggttttcttgatccgcgtatgacgaagatggcaaggatcaacaggaaaaacgccatgatgagatcaagaaacaactctagccctaattctagtccaagtgagttggttgattcaaagagacagctgatgatgcttaacttgaaaaataacgtgcagatctccgacaagaaagatagctaccaacagtccacatttgataacaagaagcttagggttttgtgtgagaaggaattgaagaacagcgatgttgggtcactcgggaggatagttctaccaaagagagatgcagaagcaaatcttccgaagctatctgataaagaaggaatcgttgtacagatgagagatgttttctctatgcagtcttggtctttcaaatacaagttttggtccaataacaagagcagaatgtatgtcctcgagaacacaggagaatttgtgaagcaaaatggagctgagataggagactttttaacaatatacgaggacgaaagcaagaatctctacttcgccatgaatggaaattcgggaaaacaaaatgaaggaagagaaaatgagtcgagggaaaggaaccactacgaagaggcaatgcttgattacataccaagagacgaagaggaagcttccattgcaatgctcatcggaaatctaaacgatcactatcccatccctaacgatctcatggacctcaccactgaccttcagcaccatcaagccacgtcctcatcaatgccacctgaggatcacgcgtacgtgggttcatccgatgatcaggtgagctttaacgactttgagtggtggtgaggtaccctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaggcgcgcccgggattaagacccgtcgtcaagtaggaattccagaaatcaacgcacggtaactaaatcctcgcgctaagctgggtcagcgctacccagctttcttgtacaaagtggggatcggccagaatggcccggaccgggttaccaagcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggtgtccaagggggaggagctcattaaagagaacatgcacatgaagttatacatggagggcaccgtgaataatcaccacttcaagtgtacctctgagggtgagggcaagccctatgagggaacccagactatgagaatcaaggtggtcgaaggaggtccactcccattcgccttcgacatactagccacctccttcatgtacgggagtagaaccttcatcaaccacacccagggcatcccagacttcttcaaacagtcattcccagagggattcacctgggagagagtgaccacctacgaagacggaggagtcttgaccgccacccaagacactagtcttcaagacggctgcttgatctacaacgtcaagatccgcggcgtgaacttcccatcaaacggtcccgtgatgcagaagaagactctcggctgggaagccaatactgaaatgctttacccagccgacggtggtcttgaaggtagatccgacatggccttgaagcttgtcggcggaggccatctcatctgtaacttcaagaccacctaccgctccaaaaagcctgccaaaaacttgaaaatgcccggcgtgtattacgtggaccatagactcgagcgcataaaagaggccgacaaggagacttatgtggagcagcacgaggtggccgtcgctagatattgcgacttgccatcaaagcttggccacaaactgaattaggcgatcgctagatctttgagctcttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggttaaccccggggtcggaggagactagccgttagttagtgcagaccgtctctaatcagtgactagcgacgtcccgacccacccagctttcttgtacattcaaagccaatgactgttgttaggtccagtttctaaggacgaatcatccaatcactggttctttctgaacaaaacaaagagctatagagttacgttactgtagccgagacagagcctgcaatctggttatgccacatactaggttctttcttaaagttgactcactctatcctaagttctacttacaaggagctacaacaaattggtcataaccggagagcgttcaaggaggtaatatcccatcttactctgtagcaaggctacaaaaggagagctaaagaactaggctacaaaaagagagctaaagaactaactatctagcaagactacagagctatagtgtagagatttcataaccggtgagcgttcaaggatactttgaagagaagaggtaatatctcaactagcaagactacaaaaaaatggagatataaaactaactgtctaacaagactacagagctatagtagagatagatacgttcgacacttaagcatgaggtgaccaagcttggcgcgcccagggcccatcgatatcggtaccctgggatccgatatcttattagtgtttgtgcaaattcgactctacttgagttaggtagacagattcatggattgagtattaagtctagctttgattcttctagctttgtgggaagttctttagtctcgttgtattcaaagtgtggtgttcctgaaggagcttatcaagtttttaacgaggtgcctgtgaagaatctcgggatttggaacgctatgctcaaggcgtatgcgcaacattcacatactcaaaaggttattgaattgttcaaaaggatgaagctttctggaatgaaaccaaatttcataacttttttgaatgtgctcaatgcttgtagtcacgcgggtctagttgatgaagggagatactactttgatcagatgaaagaatcgaggattgagccgacggataagcattacgcttccttggtggatatgttaggacgagctggtagattacaagaagcacttgaagttatcactaatatgccgattgatcccacggaatctgtatggggagctttattaacgagttgtacagtccacaagaacacagagcttgcagcatttgcagcagacaaggtcttcgaattaggaccagtgagttccggtatgcacatttcgttatccaatgcttatgcagccgatggtagattcgaagacgcagctaaagctagaaaattgcttcgagaccgaggggaaaagaaggaaacagggctaagttgggttgaagaaaggaacaaggttcatacatttgcagctggagaaagacgtcatgagaaaagcaaggagatatatgagaagctggctgagttaggggaagagatggagaaagcgggttacattgcagacacaagctatgttctgagagaagtggatggtgacgagaagaatcaaaccataaggtatcatagtgagagattagctattgcttttggactgatcacgtttccagctgatagaccgatccgagtaatgaagaatctgcgtgtttgcggtgattgccataacgcgatcaagttcatgtccgtatgtacgagacgagtgatcattgtgagagataataatcggtttcatcggtttgaagatggcaagtgttcttgtaatgactattggtgaagaagacaaatgagagttggtttatatttaaccataatttcattcagttcacactgaaccggcgaaatttctttgccagacctattcggaattgaaacaagtggagtctcgaaacgaaaagaactttctggaattcgtttgctcacaaagctaaaaacggttgatttcatcgaaatacggcgtcgttttcaaagaacaatccagaaatcactggttttcctttatttcaaaagaagagactagaactttatttctcctctataaaatcactttgttttttcctctcttcttcataaatcaacaaaacaatcacaaatctctcgaaacgctctcgaagttccaaattttctcttagcattctctttcgtttctcgtttgcgttgaatcaaagttcgttgcgggatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtaccgttaacagatctactaactatctatactgtaataatgttgtatagccgccggatagctagctagtttagtcattcagcggcgatgggtaataataaagtgtcatccatccatcaccatgcgtggcaacgtgagcaatgacctgattgaacaaattgaaatgaaaagaagaaatatgttatatgtcaacgagatttcctcataatgccactgacgacgtgtgtccaagaaatgtatcagtgatacgtatattcacaatttttttatgacttatactcacaatttgtttttttactacttatactcacaatttgttgtgcgtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgcttaagcttcggtccgggcctagaaggccgatctcccgggcaactttattatacaaagttgatcacgtgagagtctaaaaacggcccctaactaacgcttagttgcccgctagattagtcccccgttagtgcctagttagacacacgacgaactaggggctataaggatttaaatatggtacggatccgtcgacggcgcgccgcggacctgaattccagcacactggcggccgttaataacttcgtatagcatacattatacgaagttatagtggcatcacgtttctagttctagacccatcagctgaggtacaaattacggccagaatggccatctggattcagcagggggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagccatggctgcaactactcttacatctgctttacctggtgctttcagttcatcccagagaccaagtgctcctttcaacttacaaagatcacctagagtgcttagaaggttcaacagaaagacaggacgtcaacctcgtggacttgttcgtgctgcaaaggctcaagcggcgcaaaacctgccagagaaattcgatgaaacaagactctgggaggccctcaggggatttggggttagtccaagtagggttacttacgctcccgtcggattcggagattaccactggaccgtcacagatgaagacgggagaccttggttcgcaactgtcagtgacctcgaacacaaagagcattgcgggcagggagcacaagcagcccttaaaggactcagacaagctatggacactgccctcgcacttagagatagggacggactcagattcgtggttgctccagtggcagcaacagatgggggtggccctgtccttccccttgacgctaggtatgcactcacagtcttccctcacgtcccaggaagaacaggagagttcggacaaagactcacagaggcagagagggacagactgcttgctcttctggcagaactccacggaagaacacctcctgaaacaaccccaccagccgatatggaaccaccaggacttcctggtgttagagcagcactcgccgaaagtgaaggaccttggagtggaggaccatttgcagaacccgctagacttcttctcagagaacacgaggccacactgcacgcaagactcgctgaattcgaagccctcgtcgcaagagttaagggtcgtggagcacctcttgtcgttacccacggagaaccacacccaggcaacttgatcctcggagaggaaggctatctcctcgtggactgggatacagttggactggctccagctgaaagggatttgagtctcatctcagacgacccagctgacctcgcaagatacgcagagcttacaggaagaactccagacccagacgcactcgcactctatagactgcgttggagtctccttgatgtggctgagttcgtcgagtggttcagggctgaacaccaaagaacccaagacaccgagagtgcatggaaggggttcactgacacacttgggcaattggctgctggtggatcagctgcatagcaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagcggccgcaattggatcctgaggtacaaattaattaataagtgactagggtcacgtgaccctagtcacttatgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctagctagctaagtttaaacggccggccaaacgtgcaagcgctactagacaattcagtacattaaaaacgtccgcaatgtgttattaagttgtctaagcgtcaatttatttacaattgaatatatcctgcca

SEQ ID NO: 68 - DNA, 510 bp, Brassica napus
  source 1..510: mol_type = other DNA
atgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgtttag

SEQ ID NO: 69 - protein, 169 aa, Brassica napus
MSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNV

SEQ ID NO: 70 - DNA, 297 bp, Arabidopsis thaliana
  source 1..297: mol_type = other DNA
acatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaa

SEQ ID NO: 71 - protein, 98 aa, Arabidopsis thaliana
TCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 72 - DNA, 137 bp, synthetic construct
  misc_feature 1..137: note = Promoter
  source 1..137: mol_type = other DNA
ccttttgtctcccttttgtctcccttttgtctcccttttgtctcccttttgtctcccttttgtctcccttttgtctcccttttgtctccgcaagacccttcctctatataaggaagttcatttcatttggagaggac

SEQ ID NO: 73 - DNA, 855 bp, Rattus norvegicus
  source 1..855: mol_type = other DNA
acaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatga

SEQ ID NO: 74 - protein, 284 aa, Rattus norvegicus
TKKKIKGIQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQK

SEQ ID NO: 75 - DNA, 2133 bp, Glycine max
  source 1..2133: mol_type = other DNA
atggggtctatgaatttgttaggtttttctctctctcctcaagaacacccttctagtcaagatcactctcaaacggcaccttctcgtttttgcttcaaccctgatggaatctcaagcactgatgtagcaggagactgctttgatctcacttctgactcaactcctcatttactcaaccttccctcttacggcatatacgaagcttttcataggagcaacaatattcacaccactcaagattggaaggagaactacaacagccaaaacttgctattgggaacttcatgcagcaaccaaaacatgaaccacaaccatcagcaacaacaacaacaacagccaaagcttgaaaacttcctcggtggacactcatttggtgaacatgagcaaccctacggtggtaactcagcctctacagaatacatgttcccggctcagccggtattggccggtggcggcggcggtggtagcaatagcagcaacacaagcaacagtagctccatagggttatccatgataaagacatggttgaggaaccaaccaccacactcagaaaacaacaataacaacaacaatgaaagtggtggcaatagtagaagcagtgtgcagcagactctatcactttccatgagtactggttcacaatcaagcacgtcactaccccttctcactgctagtgtggataatggagagagttcttctgataacaaacaaccacataccacggctgcacttgatacaacccaaaccggagctattgaaactgcacccagaaagtctattgacacttttggacagagaacttctatctaccgtggtgtaacaaggcataggtggacggggaggtatgaggctcacctgtgggataatagttgtagaagagagggacaaactcgcaaaggaaggcaagtttacttgggaggttatgacaaagaagaaaaggcagctagagcctacgatttggcagcactaaaatactggggaacaactacgacaacaaattttccaattagccactatgagaaagagttggaagaaatgaagcacatgactaggcaagagtacgttgcgtcattgagaaggaagagtagtgggttttctcgcggggcatccatttatcgaggtgtgacgagacaccatcaacatggaagatggcaagcgaggattggaagagttgctggcaacaaggatctctacttgggaactttcagcacccaagaggaggcagcagaagcatacgatgtagcagcaatcaaattcagaggactaagtgctgttacaaactttgacatgagcagatatgacgtgaaaagcatacttgagagcaccactttgccaattggtggtgctgcaaagcgtttgaaggatatggagcaggtggaactgagggtggagaatgttcatagagcagatcaagaagatcatagtagcatcatgaactctcacttaactcaaggaatcattaacaactacgcagcaggaggaacaacagcgactcatcatcataactggcacaatgctcttgcattccaccaacctcaaccttgcaccaccatacactacccttatggacaaagaattaattggtgcaagcaagaacaagacaactctgatgcctctcactctttgtcttattcagatattcatcaactacagctagggaacaatggcacacacaacttctttcacacaaattcagggttgcaccctatgttaagcatggattctgcttccattgacaatagctcttcatctaactctgttgtttatgatggttatggaggtggtgggggctataatgtgattcctatggggactactactactgttgttgcaaatgatggtgatcaaaatccaagaagcaatcatggttttggtgataatgagataaaggcacttggttatgaaagtgtgtatggttctacaactgatccttatcatgcacatgcaaggaacttgtattatcttactcaacagcaaccatcttctgttgatgcagtgaaggctagtgcatacgatcaaggatctgcatgcaatacttgggttccaactgctattccaactcacgcaccaaggtctagtactagtatggctctctgccacggtgctacgcccttctctttattgcatgaatag

SEQ ID NO: 76 - protein, 710 aa, Glycine max
MGSMNLLGFSLSPQEHPSSQDHSQTAPSRFCFNPDGISSTDVAGDCFDLTSDSTPHLLNLPSYGIYEAFHRSNNIHTTQDWKENYNSQNLLLGTSCSNQNMNHNHQQQQQQQPKLENFLGGHSFGEHEQPYGGNSASTEYMFPAQPVLAGGGGGGSNSSNTSNSSSIGLSMIKTWLRNQPPHSENNNNNNNESGGNSRSSVQQTLSLSMSTGSQSSTSLPLLTASVDNGESSSDNKQPHTTAALDTTQTGAIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELRVENVHRADQEDHSSIMNSHLTQGIINNYAAGGTTATHHHNWHNALAFHQPQPCTTIHYPYGQRINWCKQEQDNSDASHSLSYSDIHQLQLGNNGTHNFFHTNSGLHPMLSMDSASIDNSSSSNSVVYDGYGGGGGYNVIPMGTTTTVVANDGDQNPRSNHGFGDNEIKALGYESVYGSTTDPYHAHARNLYYLTQQQPSSVDAVKASAYDQGSACNTWVPTAIPTHAPRSSTSMALCHGATPFSLLHE

SEQ ID NO: 77 - DNA, 2085 bp, Papaver somniferum
  source 1..2085: mol_type = other DNA
atggcttcttcaatgaacaactggttagggttttcattatctccgcaagaagtagtaccaacacaatcacaaactcatcatcatgcatcaaatgtctcgagacatgggtttaactctgatgaagtttctggttctgaagttgtatctagtgattgtttcgatcttggttctcacgattcttcaggtccaaccggtgcttatggcatcttagaagcttttaacagaaaccatcagcaacaacatgattggagcggcaataacttcaagtcaagttctgaactttctatgttaatgggtggatctaataataatcagcatcagcatcatttgagtcatcaagaagaaccaaagttagaagatttcttaggaggacattctttttctgaccatgatcaacagaaacttcacggtgttgccaataacaacaacaacaataataacaatggttccctggggttgtcaatgatcaaaacctggttaagaaatcaacctgcaccgccccaacaagaaaacaaagatgaggttggaaatagtacttgtggtggcggtggtggtgttggtgatggtgtggtgggaagtaatggacaaacactatccttatcaatgagtacaggatcacaatctagttctccagctttgccgcttgtaacgaccgcgtcaacggttgttacaggtggtggtggtggtagtggtggtgctgaaagttcttcgtcggagaataacaagcaaaagactaatggtactactacggctctggatagtcctactgctggtgcaatagtagaagcagttccaaggaaatcaattgatactttcggacagagaacttctatctacagaggtgtaactaggcatagatggacaggcagatatgaagcccatctttgggataatagttgcagaagagaaggtcagactagaaagggtagacaagtttatttgggtggttatgacaaagaagataaggctgctagggcttatgatttggctgccttgaaatattggggatcaaccaccactacaaactttccgattgctaattacgagcaagagttggaagatatgaagaacatgactagacaggaatatgtagcatcacttagaaggaaaagcagtggtttttctcgtggagcatcaatttatcgcggtgtcaccaggcaccatcagcacggtagatggcaagcaagaattggaagggttgcagggaacaaggatttgtacttgggcacattcagtacccaagaagaagcagctgaggcctacgacatagctgcgattaagttccgtggtttgaatgcagtaacaaatttcgatatgagcagatacgatgtaagtagcatcatggaaagtagtacattgccgatcggaggcgctgcaaaacgtttaaaagaagctgagcaaattgcagaagcaagtgttgatgctcgaagagcagagagtgataaccttacttcacagttaaccaacggaatcaacaactacggatggccaactattgctttccaacaagctcagcctattagcatgctgtatccatacaatcaacaaagatctgggtggtgtaaacaggaacaagaagcggctgctgctgctcatagctttcaagatcttcatcagcttcaattgggtagtacccatcataacttttttcaaccttctgttcttcataatctcatgagccaagattcctcttcactggaacatagttctggttccaattctgttatttataatggcggaggtggaactggaagtggtgcaagtaatgggagttatcaaatgggtggaagtaatggctatctgccaatgggaatggttatgagtagtgagcatcagaacaatccaactggaagtagtttcgtggagaatgacttgaaacaacaaatgggatatgatagtattatgtcttccaacagtacagatccttatagtcaaggaagaaatgcttattacatgtcgcaacaatcacctccggcttgcaataactggatgcctactggagttcaagctttagcaccgaggtctaataatctcgctgtttgtcatggtgctccgtcgacatttacggtttggaatgatgcataa

SEQ ID NO: 78 - protein, 694 aa, Papaver somniferum
MASSMNNWLGFSLSPQEVVPTQSQTHHHASNVSRHGFNSDEVSGSEVVSSDCFDLGSHDSSGPTGAYGILEAFNRNHQQQHDWSGNNFKSSSELSMLMGGSNNNQHQHHLSHQEEPKLEDFLGGHSFSDHDQQKLHGVANNNNNNNNNGSLGLSMIKTWLRNQPAPPQQENKDEVGNSTCGGGGGVGDGVVGSNGQTLSLSMSTGSQSSSPALPLVTTASTVVTGGGGGSGGAESSSSENNKQKTNGTTTALDSPTAGAIVEAVPRKSIDTFGQRTSIYRGVTRHRWTGRYEAHLWDNSCRREGQTRKGRQVYLGGYDKEDKAARAYDLAALKYWGSTTTTNFPIANYEQELEDMKNMTRQEYVASLRRKSSGFSRGASIYRGVTRHHQHGRWQARIGRVAGNKDLYLGTFSTQEEAAEAYDIAAIKFRGLNAVTNFDMSRYDVSSIMESSTLPIGGAAKRLKEAEQIAEASVDARRAESDNLTSQLTNGINNYGWPTIAFQQAQPISMLYPYNQQRSGWCKQEQEAAAAAHSFQDLHQLQLGSTHHNFFQPSVLHNLMSQDSSSLEHSSGSNSVIYNGGGGTGSGASNGSYQMGGSNGYLPMGMVMSSEHQNNPTGSSFVENDLKQQMGYDSIMSSNSTDPYSQGRNAYYMSQQSPPACNNWMPTGVQALAPRSNNLAVCHGAPSTFTVWNDA

SEQ ID NO: 79 - DNA, 2133 bp, Zea mays
  source 1..2133: mol_type = other DNA
atggcaacagtcaacaactggctagcattcagtctaagtccccaagaattacctccatcacaaactaccgatagcacacttatctctgcagcaactgccgatcatgttagcggagatgtgtgttttaacataccacaagactggagcatgagaggatctgaactatcggcattagttgcagaaccaaaactcgaggatttcctaggtggaatctctttcagcgaacaacaccataaagccaactgcaacatgataccaagcacctcttctacagtatgctacgcatctagcggagctagcactggataccaccaccaactctatcaccaaccaacatcttcagcactacactttgcagatagtgtaatggtcgcaagttcagctggagtacatgatggaggagctatgttatcagcagcagcagcaaacggagtagcaggagctgcgtctgcaaatggaggaggaataggactatccatgatcaagaactggctaagatcacagccagcacccatgcaacctagagtagcagccgcagagggagctcaaggtttatctctaagcatgaatatggccggaacaacccaaggagccgcaggcatgccattattggcaggagaaagagccagggcacctgaatcagtatcaacaagcgcacaagggggagcagtagttgttactgctccaaaagaagattcaggcggaagcggagtagcaggtgcactagtagctgtttctacagatacaggtggatctggaggagcctctgcagataacacagcaagaaagacagtagacacatttggtcagagaacatccatctacaggggagtaacaaggcacagatggacaggaagatacgaagcacacctatgggataatagttgtagaagagaagggcaaacaagaaagggaaggcaagtatacttgggtggttacgacaaagaggagaaggcagctagagcttatgatctagctgcacttaagtactggggagcaaccaccacaacaaacttcccagtcagtaactacgagaaagagctcgaggacatgaagcacatgacaagacaagagttcgtggcaagccttagacgtaaaagctcaggattttcaaggggagcctcaatttaccgtggagtcactagacatcatcaacatggaagatggcaagcaagaataggaagagtggcaggaaacaaagatctgtacctaggaaccttttccacacaagaagaagcagcagaagcatacgacattgctgcaataaagtttagggggctaaacgcagttaccaacttcgacatgagcagatacgacgtaaaaagcatcctcgactctagtgctttacctataggaagcgcagcaaaaagactaaaagaggcagaggcagcagcatcagcacagcatcatcatgcaggtgttgtaagctatgatgtcggaagaatagcaagccagcttggagacggaggagctttggcagcagcatatggcgctcattatcatggagcagcatggccaacaatagcattccaaccaggagctgctagcacaggactatatcacccatacgcacaacaaccaatgagaggaggaggatggtgcaaacaagaacaggaccacgcagtaatagcggcagcccacagcctacaagatttgcaccacttgaatcttggagcagcaggagcacatgacttcttcagtgcaggtcaacaagcagctgctgcagctatgcacggattaggtagtatagacagtgcctcactagaacactcaacaggatcaaacagcgtcgtatacaacggaggagtcggggattctaatggagcatctgctgtaggaggaagtggaggtggttacatgatgccaatgagcgcagcaggagcaacgacaacaagcgctatggtaagtcacgaacaagtacatgctagagcatacgatgaagctaaacaagcagcccaaatgggatacgaaagctaccttgtaaacgccgagaacaatggaggaggaaggatgtccgcatggggaaccgtggtttcagcagctgctgcagctgcagcaagttccaatgataacatggcagcagatgttggacatggaggggctcaactcttctccgtctggaacgatacataa

SEQ ID NO: 80 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 81 - DNA, 490 bp, Arabidopsis thaliana
  source 1..490: mol_type = other DNA
cttgcctcaattccggaggtgtttctagtgttcaacatgacaaacaaaacccatctctttcagtatatgtctctcagttgtgcttaattcaaatttcaactcagagaacttcttggcatacttatccagattatctaatgatctcatctaatggtaattcaactttcagtatatgtctcgcagcaaactatctttacatcaaatttttaacaactcaatgcacaaaatacttttcctcaacctaaaaataaggcaattagccaaaaacaactttgcgtgtgaacaacgcgttacacgtccctacacatacgtgtcaatttataattggctattgcttccacgccttagctttctcgtgaccgaccgagtcgtcctcgtcttttttgcttctataaatcaaatacccaaacacctcttcttcttcacaattcagattccaattttctcaaactctaaaatcaatctctcaaatctctcaaccgtgatcaag

SEQ ID NO: 82 - DNA, 443 bp, Cassava vein mosaic virus
  source 1..443: mol_type = other DNA
ccagaaggtaattatccaagatgtagcatcaagaatccaatgtttacgggaaaaactatggaagtattatgtgaggtcagcaagaagcagatcaatatgcggcacatatgcaacctatgttcaaaaatgaagaatgtacagatacaagatcctatactgccagaatacgaagaagaattcgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaac

(19) United States
(12) Patent Application Publication
(10) Pub. No.: US 2025/0270265 A1
(43) Pub. Date: Aug. 28, 2025

(54) COMPOSITIONS AND METHODS FOR SOMATIC EMBRYOGENESIS IN DICOT PLANTS

(71) Applicant: Pioneer Hi-Bred International, Inc., JOHNSTON, IA (US)

(72) Inventors: HYEON-JE CHO, ANKENY, IA (US); WILLIAM JAMES GORDON-KAMM, URBANDALE, IA (US); TODD J. JONES, JOHNSTON, IA (US); NAGESH SARDESAI, JOHNSTON, IA (US); UYEN CAO CHU, URBANDALE, IA (US); YINGHONG LI, URBANDALE, IA (US)

(73) Assignee: PIONEER HI-BRED INTERNATIONAL, INC., JOHNSTON, IA (US)

(21) Appl. No.: 18/859,791

(22) PCT Filed: Apr. 25, 2023
(86) PCT No.: PCT/US2023/066179

§ 371 Date: Oct. 24, 2024

Related U.S. Application Data
(60) Provisional application No. 63/335,488, filed on Apr. 27, 2022.

(51) Int. Cl. C07K 14/415; C12N 15/82

(52) U.S. Cl. CPC C07K 14/415; C12N 15/8205

(57) ABSTRACT

Methods are provided for producing dicot somatic embryos, including for transformation and regenerating transgenic plants from the somatic embryos.

### CROSS-REFERENCE TO RELATED APPLICATIONS

This application claims priority to U.S. Provisional Application No. 63/335,488, filed Apr. 27, 2022, which is hereby incorporated herein by reference in its entirety.

### REFERENCE TO A SEQUENCE LISTING SUBMITTED ELECTRONICALLY

The official copy of the sequence listing is submitted electronically concurrently with the specification as an xml formatted sequence listing file named 8761-WO-PCT.ST26, created on Apr. 24, 2023, and having a size of 379,276 bytes, which is part of the specification and is incorporated herein by reference in its entirety.

### FIELD OF THE DISCLOSURE

The present disclosure relates generally to the field of plant molecular biology, including tissue culture and genetic techniques in plants. The present disclosure also provides compositions and methods for the rapid, highly efficient production of dicot somatic embryos and transformation of dicot plants.

### BACKGROUND

Methods for generating somatic embryos in dicot plants can be difficult, laborious, and time-consuming. Further, many commercially relevant varieties of dicot plants and inbred lines are recalcitrant to traditional culture methods for somatic embryogenesis. Thus, only a limited range of dicot plant genotypes are capable of generating somatic embryos using traditional culture methods.

Despite its limitations, Agrobacterium-mediated transformation remains a widely used approach in experimental and, in some cases, commercial development of dicot species such as cotton, canola, alfalfa, tomato, and cucumber. To date, the most common methods used for contacting cells with Agrobacterium include: culturing explant tissue such as immature embryos (“co-culture”), possibly including a “delay” or “resting” (non-selective) step, followed by culturing on selection medium containing auxin(s) allowing de-differentiation of cells to form callus. During this callusing phase, transformed resistant callus tissue is selected in the presence of an appropriate selection agent on a selection medium. This is followed by growth of cells under conditions that promote differentiation of the callus and regeneration of the callus into plants on regeneration and rooting media. However, these traditional methods are subject to a range of challenges in practice.

For example, in cotton, traditional methods of generating somatic embryo tissue, transforming that tissue, and regenerating a transgenic plant that can be transferred to soil for further growth can take 4-5 months and are often quite inefficient. In another example, canola transformation protocols regularly regenerate plants that are malformed due to hydrophobicity and poor-quality shoot formation. Chu et al. (2020) Frontiers in Plant Sciences 11 (579524): 1-11. Further, these methods tend to be very labor intensive.

There is a desire for improved, simpler, quicker, and more efficient methods of making dicot somatic embryos for research and product development applications. There is also a need for new methods capable of transforming plant varieties and inbreds that are recalcitrant to established transformation protocols. Such alternative methods could save time and provide greater flexibility in making research and product development decisions. Such methods could also be used to facilitate higher throughput transformation and/or expand the pool of plant materials available for transformation by enabling the creation of a larger number and/or wider range of transgenic plants for gene testing and/or product development in a manner that lowers material and labor costs.

### SUMMARY OF THE DISCLOSURE

The compositions and methods disclosed herein are based, at least in part, on the surprising discovery that combinations of two or more heterologous morphogenic genes can be used to induce somatic embryo formation in dicot explants. In some examples, the disclosed compositions and methods induce the formation of somatic embryos, in dicot plant species and/or types of dicot explants that are recalcitrant to somatic embryo formation using established methods. In some examples, the disclosed compositions and methods induce the formation of dicot somatic embryos in a simpler, faster, more cost-effective, and/or more readily scalable high-throughput manner relative to established methods. In some examples, the disclosed compositions and methods induce the formation of dicot somatic embryos which are used to generate transgenic plants in dicot plant species, dicot plant lines and/or in types of dicot explants that are recalcitrant to somatic embryo formation (or regeneration of transgenic plants from somatic embryo tissue) using established methods.

In one aspect, provided herein is a method of producing dicot somatic embryos that includes expressing in a dicot explant: (i) a first heterologous nucleotide sequence encoding a Babyboom (BBM) or a RWP-RK domain (RKD) polypeptide and (ii) a second heterologous nucleotide sequence encoding a leafy cotyledon (LEC) polypeptide. As shown in the Examples herein, the combined expression of these morphogenic polypeptides has been surprisingly found to induce somatic embryo formation in a representative number of dicot species, including some dicots that, either fail to respond or respond much less efficiently to established techniques for inducing somatic embryogenesis. In one example of this method, the first heterologous nucleotide sequence encodes a BBM polypeptide and the second heterologous nucleotide sequence encodes a LEC2 polypeptide. In another example of this method, the first heterologous nucleotide sequence encodes a BBM polypeptide and the second heterologous nucleotide sequence encodes a LEC1 polypeptide. In a further example of this method, the first heterologous nucleotide sequence encodes a RKD4 polypeptide and the second heterologous nucleotide sequence encodes a LEC1 polypeptide. In yet another example of this method, the first heterologous nucleotide sequence encodes a RKD4 polypeptide and the second heterologous nucleotide sequence encodes a LEC2 polypeptide.

The dicot somatic embryos generated by the methods disclosed herein can be used in a transformation method to generate transgenic plants or plant tissues. The somatic embryo can be used for transient or stable transformation. Transient or stable transformation can be done by Agrobacterium- or biolistic-based methods.

In a second aspect, provided herein is a method for producing a transgenic dicot plant. This method includes: (i) transforming a dicot plant explant with a first heterologous nucleotide sequence encoding a BBM polypeptide or a RKD polypeptide and a second heterologous nucleotide sequence encoding a LEC polypeptide; (ii) transforming the explant with a transgene; (iii) inducing somatic embryo formation in the explant to produce a transgenic somatic embryo; and (iv) culturing the transgenic somatic embryo tissue under germination, rooting, and shooting conditions to form a transgenic dicot plant comprising the transgene. In one example of this transformation method, the first heterologous nucleotide sequence encodes a BBM polypeptide and the second heterologous nucleotide sequence encodes a LEC2 polypeptide. In another example of this transformation method, the first heterologous nucleotide sequence encodes a BBM polypeptide and the second heterologous nucleotide sequence encodes a LEC1 polypeptide. In yet another example of this transformation method, the first heterologous nucleotide sequence encodes a RKD4 polypeptide and the second heterologous nucleotide sequence encodes a LEC1 polypeptide. In a further example of this transformation method, the first heterologous nucleotide sequence encodes a RKD4 polypeptide and the second heterologous nucleotide sequence encodes a LEC2 polypeptide. Additionally, in each of the foregoing examples of the transformation method, the method can further include excising the first and second heterologous nucleotide sequences encoding the morphogenic polypeptides (BBM or RKD and LEC1 or LEC2) following somatic embryo induction.

For example, the first and second heterologous nucleotide sequences encoding a morphogenic polypeptides discussed herein (BBM, BBM AP2 domain, RKD, LEC1, LEC2, or FUS3) can be flanked by recognition sequences for a site-specific recombinase, and the disclosed transformation method can include, following embryo induction, providing the explant with the site-specific recombinase that recognizes the recognition sites and can excise the flanked coding sequences for the morphogenic polypeptides. In particular examples, one or more site-specific recombinase flanked morphogenic polypeptide (e.g., the BBM, BBM AP2 domain, RKD, LEC1, LEC2, or FUS3) is coupled to a second polypeptide domain (e.g., CAAT-Binding Factor 1A Activation Domain (CBF1A) or Glucocorticoid Receptor (GR)).

In certain examples, the disclosed method of dicot transformation advantageously reduces the time normally required to generate the transgenic dicot plant. The time to generate a T0 plant can be reduced by weeks or months following transformation with the first and second heterologous nucleotide sequences encoding the morphogenic polypeptides. For example, the time to regenerate a transgenic T0 plant can be reduced by 1, 2, 3, 4, 5, 6, 7, 8, 9, 10, 11, 12, 13, 14, 15, 16, 17, 18, 19, 20, 21, 22, 23, or 24 weeks (or more) relative to an established method for transformation of the same dicot species.

In a third aspect, provided herein is a method of producing dicot somatic embryos that includes expressing in a dicot explant a CCAAT-Binding Factor 1A Activation Domain (CBF1A) coupled to a BBM polypeptide, a LEC2 polypeptide, or both. This method includes expressing in a dicot explant (i) a first heterologous nucleotide sequence encoding a BBM polypeptide and (ii) a second heterologous nucleotide sequence encoding a LEC2 polypeptide, wherein the BBM polypeptide, the LEC2 polypeptide, or both the BBM polypeptide and the LEC2 polypeptide are coupled to a CBF1A. In one example of this method, the first heterologous nucleotide sequence encodes a BBM polypeptide that is coupled (e.g., via a linker domain) to CBF1A (BBM-CBF1A) and the second heterologous nucleotide sequence encodes a LEC2 polypeptide. In a second example of this method, the first heterologous nucleotide sequence encodes a BBM polypeptide and the second heterologous nucleotide sequence encodes a LEC2 polypeptide that is coupled (e.g., via a linker domain) to CBF1A (LEC2-CBF1A). In a third example of this method, the first heterologous nucleotide sequence encodes a BBM polypeptide that is coupled (e.g., via a linker domain) to CBF1A and the second heterologous nucleotide sequence encodes a LEC2 polypeptide that is coupled (e.g., via a linker domain) to CBF1A.

In a fourth aspect, provided herein is a method of producing dicot somatic embryos that includes expressing in a dicot explant a CBF1A coupled to a truncated version of a BBM polypeptide, where the truncated BBM comprises the BBM AP2 Domain and C-terminus, but lacks BBM N-terminus. This method includes expressing in a dicot explant a first heterologous nucleotide sequence encoding a CBF1A coupled to the truncated BBM polypeptide.

In a fifth aspect, provided herein is a method of producing dicot somatic embryos that includes expressing in a dicot explant the FUSCA3 (FUS3) gene. This method includes expressing in a dicot explant (i) a first heterologous nucleotide sequence encoding a BBM polypeptide and (ii) a second heterologous nucleotide sequence encoding a FUS3 polypeptide.

In a sixth aspect, provided herein is a method of producing dicot somatic embryos that includes expressing in a dicot explant a Glucocorticoid Receptor (GR) coupled to a BBM polypeptide, a LEC2 polypeptide, or both. This method includes expressing in a dicot explant (i) a first heterologous nucleotide sequence encoding a BBM polypeptide and (ii) a second heterologous nucleotide sequence encoding a LEC2 polypeptide, wherein the BBM polypeptide, the LEC2 polypeptide, or both the BBM polypeptide and the LEC2 polypeptide are coupled to a GR. In one example of this method, the first heterologous nucleotide sequence encodes a BBM polypeptide that is coupled to GR (BBM-GR) and the second heterologous nucleotide sequence encodes a LEC2 polypeptide. In a second example of this method, the first heterologous nucleotide sequence encodes a BBM polypeptide and the second heterologous nucleotide sequence encodes a LEC2 polypeptide that is coupled to GR (LEC2-GR). In a third example of this method, the first heterologous nucleotide sequence encodes a BBM polypeptide that is coupled to GR and the second heterologous nucleotide sequence encodes a LEC2 polypeptide that is coupled to GR. The dicot somatic embryos generated by any of the methods of the foregoing third, fourth, fifth, or sixth aspect can be used in a transformation method to generate transgenic plants or plant tissues. The somatic embryo can be used for transient or stable transformation. Transient or stable transformation can be done by Agrobacterium or biolistic-based method. In particular examples of this transformation method, the first and second heterologous nucleotide sequences encoding morphogenic polypeptides (i.e., the disclosed combination of BBM, LEC2, BBM-CBF1A, LEC2-CBF1A, BBM-GR, LEC2-GR, and/or FUS3 polypeptides) following somatic embryo induction.

The explant tissue used in any of the foregoing methods can be hypocotyl, epicotyl, cotyledon, leaf, or root. The explant can be from a plant in a dicot plant of the order Ranunculales, Fabales, Cucurbitales, Malvales, Brassicales, Solanales, Asterales, or Apiales as exemplified herein. The methods, expression cassettes, and vectors described herein, can be used to generate or derive somatic embryos in Eudicot explants in the clades Campanulids, Lamids, Malvids, or Fabids, as well as in the families Papaveraceae, Fabaceae, Cucurbitaceae, Malvacaeae, Brassicaceae, Solanaceae, Asteraceae, or Apiaceae.

Also disclosed herein are expression constructs suitable for use in the methods for generating somatic embryos and/or transformation described herein. Thus, in one aspect, the disclosure provides an expression construct comprising (a) a first promoter operably linked to a first heterologous nucleotide sequence encoding a BBM or a RWP-RK domain (RKD) polypeptide; and (b) a second promoter operably linked to a second heterologous nucleotide sequence encoding a leafy cotyledon (LEC) polypeptide. For example, the first heterologous nucleotide sequence can be SEQ ID NO:4 encoding a BBM, and the second polypepetide can be SEQ ID NO:6 encoding a LEC2 polypeptide. In another example the first heterologous nucleotide sequence can be SEQ ID NO:8 encoding a RKD polypeptide, and the second polypepetide can be SEQ ID NO:6 encoding a LEC polypeptide. The first and second promoters can be inducible promoters, developmentally regulated promoters, or constitutive promoters. The first and second promoters can be any promoter suitable for expressing the morphogenic proteins disclosed herein.

In another aspect, this disclosure provides an expression construct comprising CCAAT-Binding Factor 1A Activation Domain (CBF1A) coupled to a BBM polypeptide, a LEC2 polypeptide, or both. For example, the construct can comprise (a) a first promoter operably linked to a first heterologous nucleotide sequence encoding a BBM polypeptide that is coupled (e.g., via a linker domain) to CBF1A (BBM-CBF1A); and (b) a second promoter operably linked to a second heterologous nucleotide sequence encoding a LEC2 polypeptide. In another example, the first heterologous nucleotide sequence encodes a BBM polypeptide and the second heterologous nucleotide sequence encodes a LEC2 polypeptide that is coupled (e.g., via a linker domain) to CBF1A (LEC2-CBF1A). In yet another example, the first heterologous nucleotide sequence encodes BBM-CBF1A and the second heterologous nucleotide sequence encodes a LEC2-CBF1A. The first and second promoters can be inducible promoters, developmentally regulated promoters, or constitutive promoters. The first and second promoters can be any promoter suitable for expressing morphogenic proteins disclosed herein.

An additional aspect of the disclosure provides an expression construct comprising a truncated version of BBM comprising the BBM AP2 Domain. For example, the construct can comprise (a) a first promoter operably linked to a first heterologous nucleotide sequence encoding a BBM AP2 Domain; and (b) a second promoter operably linked to a second heterologous nucleotide sequence encoding a LEC2 polypeptide. In another example, the construct comprises (a) a first promoter operably linked to a first heterologous nucleotide sequence encoding a BBM AP2 Domain that is coupled (e.g., via a linker domain) to CBF1A (BBM AP2-CBF1A) and (b) a second promoter operably linked to a second heterologous nucleotide sequence encoding a LEC2 polypeptide.

Yet another aspect of this disclosure provides an expression construct comprising (a) a first promoter operably linked to a first heterologous nucleotide sequence encoding a BBM polypeptide and (b) a second promoter operably linked to a second heterologous nucleotide sequence encoding a FUS3.

In still another aspect, this disclosure provides an expression construct comprising a Glucocorticoid Receptor (GR) coupled to a BBM polypeptide, a LEC2 polypeptide, or both. For example, the construct can comprise (a) a first promoter operably linked to a first heterologous nucleotide sequence encoding a BBM coupled to GR polypeptide (BBM-GR); and (b) a second promoter operably linked to a second heterologous nucleotide sequence encoding a LEC2 polypeptide. In another example, the first heterologous nucleotide sequence encodes a BBM polypeptide and the second heterologous nucleotide sequence encodes a LEC2 polypeptide that is coupled to GR (LEC2-GR). In yet another example, the first heterologous nucleotide sequence encodes BBM-GR and the second heterologous nucleotide sequence encodes a LEC2-GR. The first and second promoters can be inducible promoters, developmentally regulated promoters, or constitutive promoters. The first and second promoters can be any promoter suitable for expressing morphogenic proteins disclosed herein.

Each of the foregoing expression constructs can further include recognition sites for a site-specific recombinase flanking the 5′-end and the 3′-end of a sequence comprising the first heterologous nucleotide sequence and the second heterologous nucleotide sequence (e.g., the recognitions sites can flank the sequence comparing the expression cassette that includes (a) the first promoter and first heterologous nucleotide sequence and (b) the second promoter and second heterologous nucleotide sequence. In a particular example, each of the foregoing expression constructs can further include a nucleotide sequence encoding the site-specific recombinase that recognizes and excises the intervening sequence flanked by the recognition sites. The site-specific recombinase sequence can optionally be operably linked to an inducible promoter, a developmentally regulated promoter, a constitutive promoter or any promoter suitable for expressing morphogenic proteins disclosed herein.

### BRIEF DESCRIPTION OF THE DRAWINGS AND SEQUENCE LISTING

FIG. 1 is a phylogenetic diagram showing the relative positions of the different dicot species used for transformation in the Examples described herein.

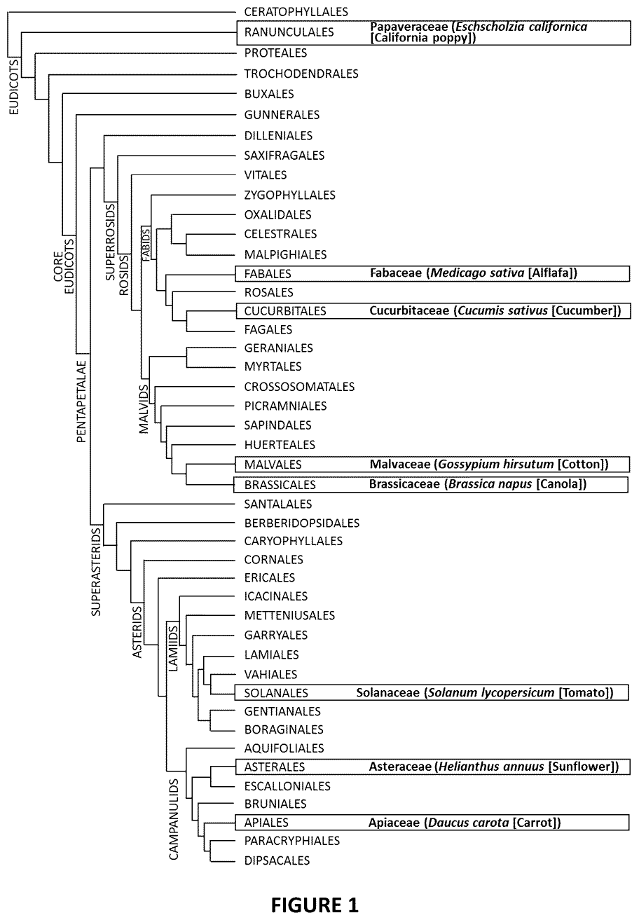

FIG. 2 is a phylogenetic diagram showing the relative positions of the different dicot species used as sources for LEC2 genes used in the Examples described herein.

FIG. 3 is phylogenetic diagram showing the relative positions of the different dicot species used as sources for BBM1 genes used in the Examples described herein.

Nucleic acid sequences listed in the accompanying sequence listing and referenced herein are shown using standard letter abbreviations for nucleotide bases. Only one strand of each nucleic acid sequence is shown, but the complementary strand is understood to be included by any reference to the displayed strand. Description of the sequence listings submitted herein are provided in Table 1.

TABLE 1

| Sequence | | |
|---|---|---|
| Listing | NAME | DESCRIPTION |
| SEQ ID NO:1 | BBM1/LEC2 | RB + LOXP + AT-UBIQ10 PRO::BN-BBM1::UBQ14 |
| | Expression | TERM + AT-UBI14 PRO::AT-UBI14 5UTR::AT-UBI14 |
| | Construct | INTRON::AT-LEC2::PHASEOLIN TERM + AT-HSP811L |
| | | PRO::MO-CRE EXON1::ST-LS1 INTRON2::MO-CRE |
| | | EXON2::SB-GKAF TERM + LOXP + GM-UBQ PRO::GM- |
| | | UBQ INTRON1::CTP::SPCN (SO)::UBQ14 TERM + GM- |
| | | EF1A2 PRO::GM-EF1A2 5UTR(1)::GM-EF1A2 |
| | | INTRON1::GM-EF1A2 5UTR(2)::DS-RED::UBQ3 TERM + LB |
| SEQ ID NO:2 | RKD4/LEC2 | RB + LOXP + AT-UBIQ10 PRO::AT-RKD4::UBQ14 TERM + |
| | Expression | AT-UBI14 PRO::AT-UBI14 5UTR::AT-UBI14 |
| | Construct | INTRON::AT-LEC2::PHASEOLIN TERM + AT-HSP811L |
| | | PRO::MO-CRE EXON1::ST-LS1 INTRON2::MO-CRE |
| | | EXON2::SB-GKAF TERM + LOXP + GM-UBQ PRO::GM- |
| | | UBQ INTRON1::CTP::SPCN (SO)::UBQ14 TERM + GM- |
| | | EF1A2 PRO::GM-EF1A2 5UTR(1)::GM-EF1A2 |
| | | INTRON1::GM-EF1A2 5UTR(2)::DS-RED::UBQ3 TERM + LB |
| SEQ ID NO:3 | RKD4/LEC1 | RB + LOXP + AT-UBIQ10 PRO::AT-RKD4::UBQ14 TERM + |
| | Expression | AT-UBI14 PRO::AT-UBI14 5UTR::AT-UBI14 |
| | Construct | INTRON::AT-LEC1::PHASEOLIN TERM + AT-HSP811L |
| | | PRO::MO-CRE EXON1::ST-LS1 INTRON2::MO-CRE |
| | | EXON2::SB-GKAF TERM + LOXP + GM-UBQ PRO::GM- |
| | | UBQ INTRON1::CTP::SPCN (SO)::UBQ14 TERM + GM- |
| | | EF1A2 PRO::GM-EF1A2 5UTR(1)::GM-EF1A2 |
| | | INTRON1::GM-EF1A2 5UTR(2)::DS-RED::UBQ3 TERM + LB |
| SEQ ID NO:4 | BN-BBM1 | Babyboom1 from Brassica napus (DNA) |
| SEQ ID NO:5 | BN-BBM1 | Babyboom1 from Brassica napus (Protein) |
| SEQ ID NO:6 | AT-LEC2 | Leafy Cotyledon2 from Arabidopsis thaliana (DNA) |
| SEQ ID NO:7 | AT-LEC2 | Leafy Cotyledon2 from Arabidopsis thaliana (Protein) |
| SEQ ID NO:8 | AT-RKD4 | RWP-RK domain containing4 from Arabidopsis thaliana |
| | | (DNA) |
| SEQ ID NO:9 | AT-RKD4 | RWP-RK domain containing4 from Arabidopsis thaliana |
| | | (Protein) |
| SEQ ID NO:10 | AT-LEC1 | Leafy Cotyledon1 from Arabidopsis thaliana (DNA) |
| SEQ ID NO:11 | AT-LEC1 | Leafy Cotyledon1 from Arabidopsis thaliana (Protein) |
| SEQ ID NO:12 | ES-LEC2 | Leafy Cotyledon2 from Eutrema salsugineum (DNA) |
| SEQ ID NO:13 | ES-LEC2 | Leafy Cotyledon2 from Eutrema salsugineum (Protein) |
| SEQ ID NO:14 | GH-LEC2 | Leafy Cotyledon2 from Gossypium hirsutum (DNA) |
| SEQ ID NO:15 | GH-LEC2 | Leafy Cotyledon2 from Gossypium hirsutum (Protein) |
| SEQ ID NO:16 | ME-LEC2 | Leafy Cotyledon2 from Manihot esculenta (DNA) |
| SEQ ID NO:17 | ME-LEC2 | Leafy Cotyledon2 from Manihot esculenta |
| SEQ ID NO:18 | CF-LEC2 | Leafy Cotyledon2 from Cephalotus follicularis (DNA) |
| SEQ ID NO:19 | CF-LEC2 | Leafy Cotyledon2 from Cephalotus follicularis (Protein) |
| SEQ ID NO:20 | TH-LEC2 | Leafy Cotyledon2 from Tarenaya hassleriana (DNA) |
| SEQ ID NO:21 | TH-LEC2 | Leafy Cotyledon2 from Tarenaya hassleriana (Protein) |
| SEQ ID NO:22 | JR-LEC2 | Leafy Cotyledon2 from Juglans regia (DNA) |
| SEQ ID NO:23 | JR-LEC2 | Leafy Cotyledon2 from Juglans regia (Protein) |
| SEQ ID NO:24 | PT-LEC2 | Leafy Cotyledon2 from Populus trichocarpa (DNA) |
| SEQ ID NO:25 | PT-LEC2 | Leafy Cotyledon2 from Populus trichocarpa (Protein) |
| SEQ ID NO:26 | AC-LEC2 | Leafy Cotyledon2 from Aquilegia coerulea (DNA) |
| SEQ ID NO:27 | AC-LEC2 | Leafy Cotyledon2 from Aquilegia coerulea (Protein) |
| SEQ ID NO:28 | AT-BBM1 | Babyboom1 from Arabidopsis thaliana (DNA) |
| SEQ ID NO:29 | AT-BBM1 | Babyboom1 from Arabidopsis thaliana (Protein) |
| SEQ ID NO:30 | HA-BBM1 | Babyboom1 from Helianthus annuus (DNA) |
| SEQ ID NO:31 | HA-BBM1 | Babyboom1 from Helianthus annuus (Protein) |
| SEQ ID NO:32 | AH-BBM1 | Babyboom1 from Amaranthus hypochondriacus (DNA) |
| SEQ ID NO:33 | AH-BBM1 | Babyboom1 from Amaranthus hypochondriacus (Protein) |
| SEQ ID NO:34 | CS-BBM1 | Babyboom1 from Cucumis sativa (DNA) |
| SEQ ID NO:35 | CS-BBM1 | Babyboom1 from Cucumis sativa (Protein) |
| SEQ ID NO:36 | TH-BBM1 | Babyboom1 from Tarenaya hassleriana (DNA) |
| SEQ ID NO:37 | TH-BBM1 | Babyboom1 from Tarenaya hassleriana (Protein) |
| SEQ ID NO:38 | JR-BBM1 | Babyboom1 from Juglans regia (DNA) |
| SEQ ID NO:39 | JR-BBM1 | Babyboom1 from Juglans regia (Protein) |
| SEQ ID NO:40 | PT-BBM1 | Babyboom1 from Populus trichocarpa (DNA) |
| SEQ ID NO:41 | PT-BBM1 | Babyboom1 from Populus trichocarpa (Protein) |
| SEQ ID NO:42 | CC-BBM1 | Babyboom1 from Cajanus cajan (DNA) |
| SEQ ID NO:43 | CC-BBM1 | Babyboom1 from Cajanus cajan (Protein) |
| SEQ ID NO:44 | GH-BBM1 | Babyboom1 from Gossypium hirsutum (DNA) |
| SEQ ID NO:45 | GH-BBM1 | Babyboom1 from Gossypium hirsutum (Protein) |
| SEQ ID NO:46 | ME-BBM1 | Babyboom1 from Manihot esculenta (DNA) |
| SEQ ID NO:47 | ME-BBM1 | Babyboom1 from Manihot esculenta (Protein) |
| SEQ ID NO:48 | CSI-BBM1 | Babyboom1 from Citrus sinensis (DNA) |
| SEQ ID NO:49 | CSI-BBM1 | Babyboom1 from Citrus sinensis (Protein) |
| SEQ ID NO:50 | VV-BBM1 | Babyboom1 from Vitis vinifera (DNA) |
| SEQ ID NO:51 | VV-BBM1 | Babyboom1 from Vitis vinifera (Protein) |
| SEQ ID NO:52 | MD-BBM1 | Babyboom1 from Malus domestica (DNA) |
| SEQ ID NO:53 | MD-BBM1 | Babyboom1 from Malus domestica (Protein) |
| SEQ ID NO:54 | SL-BBM1 | Babyboom1 from Solanum lycopersicum (DNA) |
| SEQ ID NO:55 | SL-BBM1 | Babyboom1 from Solanum lycopersicum (Protein) |
| SEQ ID NO:56 | AT-FUS3 | FUSCA from Arabidopsis thaliana (DNA) |
| SEQ ID NO:57 | AT-FUS3 | FUSCA from Arabidopsis thaliana (Protein) |
| SEQ ID NO:58 | BBM- | RB + LOXP + AT-UBIQ10 PRO::BN-BBM1-PROTEIN |
| | CBF1A/LEC2 | LINKER1-AT-CBF1A::UBQ14 TERM + AT-UBI14 |
| | Expression | PRO::AT-UBI14 5UTR::AT-UBI14 INTRON::AT- |
| | Construct | LEC2::PHASEOLIN TERM + AT-HSP811L PRO::MO-CRE |
| | | EXON1::ST-LS1 INTRON2::MO-CRE EXON2::SB-GKAF |
| | | TERM + LOXP + GM-UBQ PRO::GM-UBQ |
| | | INTRON1::CTP::SPCN (SO)::UBQ14 TERM + GM-EF1A2 |
| | | PRO::GM-EF1A2 5UTR(1)::GM-EF1A2 INTRON1::GM- |
| | | EF1A2 5UTR(2)::DS-RED::UBQ3 TERM + LB |
| SEQ ID NO:59 | BBM/LEC2- | RB + LOXP + AT-UBIQ10 PRO::BN-BBM1::UBQ14 |
| | CBF1A | TERM + AT-UBI14 PRO::AT-UBI14 5UTR::AT-UBI14 |
| | Expression | INTRON::AT-LEC2-PROTEIN LINKER1-AT- |
| | Construct | CBF1A::PHASEOLIN TERM + AT-HSP811L PRO::MO- |
| | | CRE EXON1::ST-LS1 INTRON2::MO-CRE EXON2::SB- |
| | | GKAF TERM + LOXP + GM-UBQ PRO::GM-UBQ |
| | | INTRON1::CTP::SPCN (SO)::UBQ14 TERM + GM-EF1A2 |
| | | PRO::GM-EF1A2 5UTR(1)::GM-EF1A2 INTRON1::GM- |
| | | EF1A2 5UTR(2)::DS-RED::UBQ3 TERM + LB |
| SEQ ID NO:60 | BBM1- | RB + LOXP + AT-UBIQ10 PRO::BN-BBM1-PROTEIN |
| | CBF1A/LEC2- | LINKER1-AT-CBF1A::UBQ14 TERM + AT-UBI14 |
| | CBF1A | PRO::AT-UBI14 5UTR::AT-UBI14 INTRON::AT-LEC2- |
| | Expression | PROTEIN LINKER1-AT-CBF1A::PHASEOLIN TERM + |
| | Construct | AT-HSP811L PRO::MO-CRE EXON1::ST-LS1 |
| | | INTRON2::MO-CRE EXON2::SB-GKAF TERM + LOXP + |
| | | GM-UBQ PRO::GM-UBQ INTRON1::CTP::SPCN |
| | | (SO)::UBQ14 TERM + GM-EF1A2 PRO::GM-EF1A2 |
| | | 5UTR(1)::GM-EF1A2 INTRON1::GM-EF1A2 |
| | | 5UTR(2)::DS-RED::UBQ3 TERM + LB |
| SEQ ID NO:61 | BBM AP2- | RB + LOXP + AT-UBIQ10 PRO::BN-BBM1 AP2-PROTEIN |
| | CBF1A/LEC2 | LINKER1-AT-CBF1A::UBQ14 TERM + AT-UBI14 |
| | Expression | PRO::AT-UBI14 5UTR::AT-UBI14 INTRON::AT- |
| | Construct | LEC2::PHASEOLIN TERM + AT-HSP811L PRO::MO-CRE |
| | | EXON1::ST-LS1 INTRON2::MO-CRE EXON2::SB-GKAF |
| | | TERM + LOXP + GM-UBQ PRO::GM-UBQ |
| | | INTRON1::CTP::SPCN (SO)::UBQ14 TERM + GM-EF1A2 |
| | | PRO::GM-EF1A2 5UTR(1)::GM-EF1A2 INTRON1::GM- |
| | | EF1A2 5UTR(2)::DS-RED::UBQ3 TERM + LB |
| SEQ ID NO:62 | BBM AP2/ | RB + LOXP + AT-UBIQ10 PRO::BN-BBM1 AP2::UBQ14 |
| | LEC2 | TERM + AT-UBI14 PRO::AT-UBI14 5UTR::AT-UBI14 |
| | Expression | INTRON::AT-LEC2::PHASEOLIN TERM + AT-HSP811L |
| | Construct | PRO::MO-CRE EXON1::ST-LS1 INTRON2::MO-CRE |
| | | EXON2::SB-GKAF TERM + LOXP + GM-UBQ PRO::GM- |
| | | UBQ INTRON1::CTP::SPCN (SO)::UBQ14 TERM + GM- |
| | | EF1A2 PRO::GM-EF1A2 5UTR(1)::GM-EF1A2 |
| | | INTRON1::GM-EF1A2 5UTR(2)::DS-RED::UBQ3 TERM + LB |
| SEQ ID NO:63 | ABI3 | ABI3 from Arabidopsis thaliana (DNA) |
| SEQ ID NO:64 | ABI3 | ABI3 from Arabidopsis thaliana (Protein) |
| SEQ ID NO:65 | AT-UBQ10- | RB + LOXP + AT-UBIQ10 PRO::BN-BBM1::UBQ14 |
| | BBM/AT- | TERM + AT-UBQ3 PRO::AT-UBQ3 5UTR::AT-UBQ3 |
| | UBQ3-LEC2 | INTRON::AT-LEC2::AT-UBI17414.1 TERM + GM-EF1A2 |
| | Expression | PRO::GM-EF1A2 5UTR(1)::GM-EF1A2 INTRON1::GM- |
| | Construct | EF1A2 5UTR(2)::TAGRFP::UBQ3 TERM + AT-HSP811L |
| | | PRO::MO-CRE EXON1::ST-LS1 INTRON2::MO-CRE |
| | | EXON2::SB-GKAF TERM + LOXP + GM-UBQ PRO::GM- |
| | | UBQ INTRON1::CTP::SPCN (SO)::UBQ14 TERM + LB |
| SEQ ID NO:66 | AT-UBQ10- | RB + LOXP + AT-UBIQ10 PRO::BN-BBM1::UBQ14 |
| | BBM/GM- | TERM + GM-EFTU2 PRO::AT-LEC2::AT-UBI17414.1 |
| | EFTU2-LEC2 | TERM + GM-EF1A2 PRO::GM-EF1A2 5UTR(1)::GM- |
| | Expression | EF1A2 INTRON1::GM-EF1A2 5UTR(2)::TAGRFP::UBQ3 |
| | Construct | TERM + AT-HSP811L PRO::MO-CRE EXON1::ST-LS1 |
| | | INTRON2::MO-CRE EXON2::SB-GKAF TERM + LOXP + |
| | | GM-UBQ PRO::GM-UBQ INTRON1::CTP::SPCN |
| | | (SO)::UBQ14 TERM + LB |
| SEQ ID NO:67 | AT-UBQ10- | RB + LOXP + AT-UBIQ10 PRO::BN-BBM1::UBQ14 |
| | BBM/GM- | TERM + GM-RUBACT PRO::AT-LEC2::AT-UBI17414.1 |
| | RUBACT- | TERM + GM-EF1A2 PRO::GM-EF1A2 5UTR(1)::GM- |
| | LEC2 | EF1A2 INTRON1::GM-EF1A2 5UTR(2)::TAGRFP::UBQ3 |
| | Expression | TERM + AT-HSP811L PRO::MO-CRE EXON1::ST-LS1 |
| | Construct | INTRON2::MO-CRE EXON2::SB-GKAF TERM + LOXP + |
| | | GM-UBQ PRO::GM-UBQ INTRON1::CTP::SPCN |
| | | (SO)::UBQ14 TERM + LB |
| SEQ ID NO:68 | BBM AP2 | BBM AP2 domain (DNA) |
| SEQ ID NO:69 | BBM AP2 | BBM AP 2 domain (Protein) |
| SEQ ID NO:70 | CBF1A | CCAAT-Binding Factor 1A Activation Domain (DNA) |
| SEQ ID NO:71 | CBF1A | CCAAT-Binding Factor 1A Activation Domain (Protein) |
| SEQ ID NO:72 | DR5 Promoter | Auxin-inducible synthetic promoter (DNA) |
| SEQ ID NO:73 | Glucocorticoid | Glucocorticoid Receptor from Rattus norvegicus (DNA) |
| | Receptor | |
| SEQ ID NO:74 | Glucocorticoid | Glucocorticoid Receptor from Rattus norvegicus (Protein) |
| | Receptor | |
| SEQ ID NO:75 | GM-BBM1 | Babyboom1 from Glycine max (DNA) |
| SEQ ID NO:76 | GM-BBM1 | Babyboom1 from Glycine max (Protein) |
| SEQ ID NO:77 | PS-BBM1 | Babyboom1 from Papaver somniferum (DNA) |
| SEQ ID NO:78 | PS-BBM1 | Babyboom1 from Papaver somniferum (Protein) |
| SEQ ID NO:79 | ZM-BBM1 | Babyboom1 from Zea mays (DNA) |
| SEQ ID NO:80 | ZM-BBM1 | Babyboom1 from Zea mays (Protein) |
| SEQ ID NO:81 | AT-UBI11 | Ubiquitin 11 promoter from Arabidopsis thaliana |
| | Promoter | |
| SEQ ID NO:82 | CSVMV | Promoter from Cassava vein mosaic virus |
| | Promoter | |

### DETAILED DESCRIPTION

The disclosures herein will be described more fully hereinafter with reference to the accompanying drawings, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed compositions and methods pertain having the benefit of the teachings presented in the following descriptions and the associated drawings. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” refers to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety.

Computer implementations of these mathematical algorithms can be utilized for comparison of sequences to determine sequence identity. Such implementations include, but are not limited to, CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244; Higgins, et al., (1989) CABIOS 5:151-153; Corpet, et al., (1988) Nucleic Acids Res. 16:10881-90; Huang, et al., (1992) CABIOS 8:155-65; and Pearson, et al., (1994) Meth. Mol. Biol. 24:307-331, herein incorporated by reference in their entirety. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403, herein incorporated by reference in its entirety, are based on the algorithm of Karlin and Altschul, (1990) supra. BLAST nucleotide searches can be performed with the BLASTN program, score=100, word length=12, to obtain nucleotide sequences homologous to a nucleotide sequence encoding a protein of the disclosure. BLAST protein searches can be performed with the BLASTX program, score=50, word length=3, to obtain amino acid sequences homologous to a protein or polypeptide of the disclosure. To obtain gapped alignments for comparison purposes, Gapped BLAST (in BLAST 2.0) can be utilized as described in Altschul, et al., (1997) Nucleic Acids Res. 25:3389, herein incorporated by reference in its entirety. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, the web site for the National Center for Biotechnology Information on the World Wide Web at ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. As used herein, “equivalent program” is any sequence comparison program that, for any two sequences in question, generates an alignment having identical nucleotide or amino acid residue matches and an identical percent sequence identity when compared to the corresponding alignment generated by GAP Version 10.

The GAP program uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the Quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see, Henikoff and Henikoff, (1989) Proc. Natl. Acad. Sci. USA 89:10915, herein incorporated by reference in its entirety).

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tm for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, for example, when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

### A. Expression Cassettes and Constructs for Dicot Transformation

In a first aspect, the disclosure provides morphogenic gene cassettes comprising at least two morphogenic genes that, when expressed, promote transformation and somatic embryogenesis in dicots. As used herein, “morphogenic gene cassette” and “morphogenic gene expression cassette” refer to a nucleotide construct expressing (i.e., encoding) one or both of the two morphogenic genes.

As used herein, a “morphogenic gene” refers to a gene that when expressed stimulates formation of a somatically-derived structure that can produce a plant. For example, ectopic expression of the morphogenic gene stimulates the de novo formation of a somatic embryo that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. As used herein, “transcription factor” refers to a protein that controls the rate of transcription of specific genes by binding to the DNA sequence of the promoter and either up-regulating or down-regulating expression.

The disclosure also provides expression constructs comprising the morphogenic gene cassettes as described herein. “Expression construct”, “expression vector”, and “vector” can be used interchangeably and refer to a DNA molecule such as a plasmid, cosmid, or bacterial phage for introducing a nucleotide construct, for example, an expression cassette, into a host cell. Cloning vectors can include one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance, or ampicillin resistance.

Morphogenic genes of the present disclosure include, but are not limited to, genes of the leafy cotyledon (LEC) family (e.g., LEC1 (Lotan et al., 1998, Cell 93:1195-1205) and LEC2 (Stone et al., 2008, Proc. Nat'l. Acad. Sci. USA 105:3151-3156; Belide et al., 2013, Plant Cell Tiss. Organ Cult. 113:543-553)), genes of the RWP-RK domain family (e.g., RKD4), and genes of the AP2/EREBP family (e.g., BBM (also known as ODP2)).

In some aspects, morphogenic gene cassettes comprise a first heterologous nucleotide sequence encoding a first morphogenic gene and a second heterologous nucleotide sequence encoding a second morphogenic gene. As used herein, the term “heterologous” used in connection with a “nucleotide sequence”, “polynucleotide” “coding sequence”, “gene”, “protein” or “polypeptide” refers to a sequence that is not naturally occurring in the context disclosed herein or that is not operably linked to the promoter sequence disclosed herein. While this nucleotide sequence is heterologous to the context or promoter sequence presented herein, it may be homologous or native or heterologous or foreign to the plant host.

In some aspects, the morphogenic gene cassette used in the methods and constructs disclosed herein comprises a first heterologous nucleotide sequence encoding a BBM polypeptide (e.g., BBM1 or BBM2) and a second heterologous nucleotide sequence encoding a LEC polypeptide (e.g., LEC1 or LEC2). For example, the first and second heterologous nucleotide sequences can encode BBM1 and LEC1 polypeptides, respectively, BBM1 and LEC2 polypeptides, respectively, BBM2 and LEC1 polypeptides, respectively, or BBM2 and LEC2 polypeptides, respectively.

In some aspects, the morphogenic gene cassette used in the methods and constructs disclosed herein comprises a first heterologous nucleotide sequence encoding a RKD polypeptide and a second heterologous nucleotide sequence encoding a LEC polypeptide. For example, the first heterologous nucleotide sequence can encode RKD4 and the second heterologous nucleotide sequence can encode LEC1 or LEC2 polypeptide.

The term “Babyboom1” or “BBM1” are used herein to reference a gene, coding sequence, protein, or variant thereof. Babyboom and BBM1 can refer to a BBM1 gene, coding sequence, protein, or variant thereof from Brassica napus or from Arabidopsis thaliana, Helianthus annuus, Amaranthus hypochondriacus, Cucumis sativa, Tarenaya hassleriana, Juglans regia, Populus trichocarpa, Cajanus cajan, Gossypium hirsutum, Manihot esculenta, Citrus sinensis, Vitis vinifera, Malus domestica, or Solanum lycopersicum. For example, a BBM1 nucleotide sequence can be at least 90%, 91%, 92%, 93%, 94%, 95%, 96%, 97%, 98%, or 99% nucleotide sequence identity to any of SEQ ID NOs: 4, 28, 30, 32, 34, 36, 38, 40, 42, 43, 45, 48, 50, 52, 54, 75, 77, or 79. Alternatively, for example, a BBM1 protein sequence can exhibit at least 90%, 91%, 92%, 93%, 94%, 95%, 96%, 97%, 98%, or 99% amino acid sequence identity to any of SEQ ID NOs: 5, 29, 31, 33, 35, 37, 39, 41, 43, 45, 47, 49, 51, 53, 55, 76, 78, or 80.

The terms “Leafy Cotyledon2” and “LEC2” are used herein to reference a gene, coding sequence, protein, or variant thereof. Leafy Cotyledon2 and LEC2 can refer to LEC2 gene, or a variant thereof, from Arabidopsis thaliana or from Eutrema salsugineum, Gossypium hirsutum, Manihot esculenta, Cephalotus follicularis, Tarenaya hassleriana, Juglans regia, Populus trichocarpa, or Aquilegia coerulea. For example, a LEC2 nucleotide sequence can exhibit at least 90%, 91%, 92%, 93%, 94%, 95%, 96%, 97%, 98%, or 99% nucleotide sequence identity to any of SEQ ID NO:6, 12, 14, 16, 18, 20, 22, 24, or 26. Alternatively, for example, a LEC2 protein sequence can exhibit at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In one aspect, provided is a morphogenic gene cassette that comprises a first heterologous nucleotide sequence encoding the BBM1 polypeptide, or a variant thereof, from Brassica napus and a second heterologous nucleotide sequence encoding the LEC2 polypeptide, or a variant thereof, from Arabidopsis thaliana. In another aspect, provided herein is a morphogenic gene cassette that comprises the BBM1 coding sequence, or a variant thereof, from Brassica napus and the LEC1 coding sequence, or a variant thereof, from Arabidopsis thaliana.

In one aspect, provided herein is a morphogenic gene cassette that comprises a first heterologous nucleotide sequence encoding the RWP-RK domain containing4 (RKD4) polypeptide, or a variant thereof, from Arabidopsis thaliana and a second heterologous nucleotide sequence encoding the LEC2 polypeptide, or a variant thereof, from Arabidopsis thaliana. In another aspect, provided herein is a morphogenic gene cassette that comprises the RKD4 coding sequence, or a variant thereof, from Arabidopsis thaliana and the LEC1 coding sequence, or a variant thereof, from Arabidopsis thaliana.

In one aspect, provided herein is a morphogenic gene cassette that comprises the RKD4 coding sequence, or a variant thereof, from Arabidopsis thaliana and the LEC2 coding sequence, or a variant thereof, from Arabidopsis thaliana.

In one aspect, provided herein is a morphogenic gene cassette that comprises a BBM nucleotide sequence encoding SEQ ID NO:5 and a LEC nucleotide sequence encoding any of SEQ ID NOs: 7, 11, 13, 15, 17, 19, 21, 23, 25, or 27.

In another aspect, provided herein is a morphogenic gene cassette that comprises a BBM nucleotide sequence encoding any of SEQ ID NOs: 29, 31, 33, 35, 37, 39, 41, 43, 45, 47, 49, 51, 53, 55, 76, 78, or 80 and a LEC1 or LEC2 nucleotide sequence. The LEC1 nucleotide sequence can encode SEQ ID NO:11. The LEC2 nucleotide sequence can encode any of SEQ ID NOs: 7, 13, 15, 17, 19, 21, 23, 25, or 27.

In another aspect, provided herein is a morphogenic gene cassette that comprises a RKD nucleotide sequence (e.g., a sequence encoding SEQ ID NO:9) and a LEC1 or LEC2 nucleotide sequence. The LEC2 nucleotide sequence can encode any of SEQ ID NOs: 7, 13, 15, 17, 19, 21, 23, 25, or 27. The LEC1 nucleotide sequence can encode SEQ ID NO:11.

Also provided herein is a morphogenic gene cassette that comprises a first nucleotide sequence encoding a BBM polypeptide that is coupled to a CCAAT-Binding Factor 1A Activation Domain (CBF1A) (a BBM-CBF1A polypeptide) and second nucleotide encoding a LEC1 or LEC2 polypeptide. BBM protein sequence can be any of SEQ ID NOs: 5, 29, 31, 33, 35, 37, 39, 41, 43, 45, 47, 49, 51, 53, 55, 76, 78, or 80, the CBF1A domain can be SEQ ID NO: 71, and the BBM and CBF1A can be coupled by any suitable linker sequence. The LEC2 nucleotide sequence can encode any of SEQ ID NOs: 7, 13, 15, 17, 19, 21, 23, 25, or 27. The LEC1 nucleotide sequence can encode SEQ ID NO:11. The BBM and CBF1A can be coupled by any suitable linker sequence

In a different aspect, provided herein is a morphogenic gene cassette that comprises a first nucleotide sequence encoding a BBM polypeptide and second nucleotide encoding a LEC1 or LEC2 polypeptide that is coupled to CBF1A domain (e.g., SEQ ID NO:71). The first BBM nucleotide sequence can encode any of SEQ ID NOs: 5, 29, 31, 33, 35, 37, 39, 41, 43, 45, 47, 49, 51, 53, 55, 76, 78, or 80. The second nucleotide sequence can encode the LEC2 polypeptide of SEQ ID NO:7, 13, 15, 17, 19, 21, 23, 25, or 27 coupled to the CBF1A domain; or the second nucleotide sequence can encode the LEC1 polypeptide of SEQ ID NO: 11 coupled to the CBF1A domain. The LEC polypeptide and CBF1A can be coupled by any suitable linker sequence.

In a separate aspect, provided herein is a morphogenic gene cassette that comprises a first nucleotide sequence encoding a truncated BBM that retains its AP2 Domain (BBM AP2) and a second nucleotide sequence encoding a LEC1 or LEC2 polypeptide. In some examples, the AP2 Domain can be coupled to a CCAAT-Binding Factor 1A Activation Domain (CBF1A) to produce a BBM AP2-CBF1A. Thus, the provided morphogenic gene cassette can include a first nucleotide sequence encoding a BBM AP2 (e.g. SEQ ID NO:69) and a LEC2 nucleotide sequence encoding any of SEQ ID NOs: 7, 13, 15, 17, 19, 21, 23, 25, or 27. Alternatively, the provided morphogenic gene cassette can include a first nucleotide sequence encoding a BBM AP2 (e.g. SEQ ID NO:69) linked to a CBF1A (e.g., SEQ ID NO:71) and a LEC2 sequence encoding any of SEQ ID NOs: 7, 13, 15, 17, 19, 21, 23, 25, or 27. In a different example, the morphogenic gene cassette can include a first nucleotide sequence encoding a BBM AP2 (e.g. SEQ ID NO:68) and a LEC1 nucleotide sequence encoding SEQ ID NO:11; or the morphogenic gene cassette can include a first nucleotide sequence encoding a BBM AP2 (e.g. SEQ ID NO:68) linked to a CBF1A (SEQ ID NO:70) and second LEC1 nucleotide sequence encoding SEQ ID NO:11. The BBM AP2 and CBF1A can be linked by any suitable linker sequence.

In yet another aspect, provided herein is a morphogenic gene cassette that comprises a first nucleotide sequence encoding a BBM polypeptide and a second nucleotide sequence encoding FUS3 polypeptide (e.g., SEQ ID NO:57). Thus, in this gene cassette, the first nucleotide sequence can encode the BBM polypeptide of SEQ ID NO:5 and the second can encode the FUS3 polypeptide. Alternatively, in another example, the morphogenic gene cassette can include a BBM nucleotide sequence encoding any of SEQ ID NOs: 29, 31, 33, 35, 37, 39, 41, 43, 45, 47, 49, 51, 53, 55, 76, 78, or 80 and a second nucleotide sequence encoding the FUS3 polypeptide.

Each nucleotide sequence encoding a morphogenic gene can be operably linked to a constitutive promoter, an inducible promoter, a developmentally regulated promoter, or a tissue-specific promoter that allows initiation of transcription in plant tissue. Promoters suitable for expressing a morphogenic gene disclosed herein include: UBI, ACTIN, EF1A, RUBACT, EFTU2, CAB, CSVMV, OXYGEN-EVOLVING ENHANCER (OEE), AQUAPORIN (AQP), PHOTOSYSTEM I SUBUNIT D-2 (PSID), SCBV, LLDAV, EVCV, DMMV, BSV(AY) PRO, CYMV PRO FL, IN2-2, NOS, the −135 version of 35S, DR5, XVE, GLB1, OLE, LTP2, HSP17.7, HSP26, HSP18A, AT-HSP811, AT-HSP811L, GM-HSP173B, or promoters activated by tetracycline, ethamethsulfuron or chlorsulfuron, PLTP, PLTP1, PLTP2, PLTP3, SDR, LGL, LEA-14A, or LEA-D34.

In some aspects, the first and second heterologous nucleotide sequences encoding the morphogenic genes are excised following induction of somatic embryo formation in the dicot explant. In this way, expression of the morphogenic genes can be controlled by excision at a desired time post-transformation. For example, the expression cassette can be targeted for excision by a site-specific recombinase. When a site-specific recombinase is used to control the expression of the morphogenic genes, the morphogenic gene cassette further comprises appropriate site-specific excision sites flanking the nucleotide sequences to be excised. In some cases, the expression construct comprising the morphogenic gene cassette further comprises a nucleotide sequence encoding a site-specific recombinase. Alternatively, the site-specific recombinase does not need to be co-located on the same expression construct comprising the morphogenic gene cassette and, instead, the recombinase can be expressed from a different construct. Site-specific recombinases that can be used in the methods and constructs disclosed herein include FLP, FLPe, KD, Cre, SSV1, lambda Int, phi C31 Int, HK022, R, B2, B3, Gin, Tn1721, CinH, ParA, Tn5053, Bxb1, TP907-1, or U153. The site-specific recombinase can be operably linked to a constitutive promoter, an inducible promoter, a tissue-specific promoter, or a developmentally regulated promoter such as UBI, ACTIN, EF1A, RUBACT, EFTU2, CAB, CSVMV, OXYGEN-EVOLVING ENHANCER (OEE), AQUAPORIN (AQP), PHOTOSYSTEM I SUBUNIT D-2 (PSID), SCBV, LLDAV, EVCV, DMMV, BSV (AY) PRO, CYMV PRO FL, IN2-2, NOS, the −135 version of 35S, DR5, XVE, GLB1, OLE, LTP2, HSP17.7, HSP26, HSP18A, AT-HSP811, AT-HSP811L, GM-HSP173B, or promoters activated by tetracycline, ethamethsulfuron or chlorsulfuron, PLTP, PLTP1, PLTP2, PLTP3, SDR, LGL, LEA-14A, or LEA-D34.

### B. Methods of Somatic Embryo Formation and Regeneration of Transgenic Dicot Plants

In another aspect, the disclosure provides methods of inducing somatic embryo production in dicot explants by expressing the morphogenic gene cassettes described herein to promote somatic embryo formation. More specifically, the methods disclosed herein enable direct somatic embryogenesis (DSE) of explant cells. Somatic embryogenesis is a form of induced plant cell totipotency in which embryos develop from somatic cells in the absence of fertilization. As used herein, “direct somatic embryogenesis” refers to the formation of somatic embryos directly from the surface of explants without a dedifferentiation phase or callus formation.

As used herein, “somatic embryo” refers to a multicellular structure that progresses through developmental stages that are similar to the development of a zygotic embryo. In some embodiments disclosed herein, the somatic embryo is tissue capable of regenerating a transgenic plant following transformation. Somatic embryos can be characterized by the formation of globular and transition-stage embryos, formation of an embryo axis and a scutellum, and accumulation of lipids and starch. In some cases, somatic embryos can be identified and distinguished histologically from surrounding cells by their relatively high nucleus to cytoplasm ratio, a nucleus with a single large nucleolus and relatively low heterochromatin levels, the presence of fragmented vacuoles, and by their callose-containing cell walls.

In some aspects, methods of producing dicot somatic embryos comprise expressing a morphogenic gene cassette disclosed herein in a dicot explant and culturing the explant under conditions suitable for somatic embryo formation. The morphogenic gene cassettes can be any disclosed herein comprising a first nucleotide sequence encoding a first morphogenic polypeptide and a second a nucleotide sequence encoding a second morphogenic polypeptide. Thus, methods of producing dicot somatic embryos comprise expressing in a dicot explant a first heterologous nucleotide sequence encoding a BBM or a RKD polypeptide and a second heterologous nucleotide sequence encoding a LEC polypeptide (e.g., LEC1 or LEC2).

Provided herein is a method of producing dicot somatic embryos that comprises expressing in a dicot explant: a first heterologous nucleotide sequence encoding a BBM polypeptide (e.g., BBM1 or BBM2) and a second heterologous nucleotide sequence encoding a LEC polypeptide (e.g., LEC1 or LEC2) 1. For example, the method can include expressing in a dicot explant a first heterologous nucleotide sequence encoding a BBM1 nucleotide sequence encoding SEQ ID NO:5 and a second heterologous LEC nucleotide sequence encoding any of SEQ ID NOs: 7, 11, 13, 15, 17, 19, 21, 23, 25, or 27. In other examples, the method can include expressing in a dicot explant a first heterologous BBM nucleotide sequence encoding any of SEQ ID NOs: 29, 31, 33, 35, 37, 39, 41, 43, 45, 47, 49, 51, 53, 55, 76, 78, or 80 and a LEC1 or LEC2 nucleotide sequence. Thus, the method can include expressing in a dicot explant a first heterologous BBM nucleotide sequence encoding any of SEQ ID NOs: 29, 31, 33, 35, 37, 39, 41, 43, 45, 47, 49, 51, 53, 55, 76, 78, or 80 and a LEC2 nucleotide sequence encoding SEQ ID NO:7. Alternatively, the method can include expressing in a dicot explant a first heterologous BBM nucleotide sequence encoding any of SEQ ID NOs: 29, 31, 33, 35, 37, 39, 41, 43, 45, 47, 49, 51, 53, 55, 76, 78, or 80 and a LEC2 nucleotide sequence encoding any of SEQ ID NOs: 13, 15, 17, 19, 21, 23, 25, or 27. In another example, the method can include expressing in a dicot explant a first heterologous BBM nucleotide sequence encoding any of SEQ ID NOs: 29, 31, 33, 35, 37, 39, 41, 43, 45, 47, 49, 51, 53, 55, 76, 78, or 80 and a LEC1 nucleotide sequence encoding SEQ ID NO:11.

In some aspects, methods of producing dicot somatic embryos comprise expressing a morphogenic gene cassette comprising a first heterologous nucleotide sequence encoding a RKD polypeptide (e.g., SEQ ID NO:9) and a second heterologous nucleotide sequence encoding a LEC polypeptide. The LEC polypeptide can be LEC2 polypeptide of SEQ ID NO:7 or the LEC2 polypeptide of any of SEQ ID NOs: 13, 15, 17, 19, 21, 23, 25, or 27. In a different example, a first heterologous nucleotide sequence encodes RKD4 (e.g., SEQ ID NO:9), and the second heterologous nucleotide sequence encodes LEC1 (e.g., SEQ ID NO:11).

Methods for introducing morphogenic gene pairs (i.e., the first and second heterologous nucleotide sequences) into explants can vary depending on the targeted dicot or explant source. Suitable methods of introducing nucleotide sequences into explants include microinjection, electroporation, direct gene transfer, biolistic-mediated gene transfer and bacteria-mediated transformation, such as Agrobacterium-mediated transformation, and Ochrobactrum-mediated transformation.

In some aspects, a first heterologous nucleotide sequence encoding a first morphogenic gene and a second heterologous nucleotide sequence encoding a second morphogenic gene are introduced to an explant via transferred DNA (T-DNA). As used herein, “transferred DNA” or “T-DNA” refer to a portion of a Ti plasmid that is inserted into the genome of a host plant cell. Morphogenic genes of the present disclosure can be integrated into a T-DNA transfer cassette, that is, T-DNA comprising a morphogenic gene expression cassette flanked by a right border and a left border. Agrobacterium tumefaciens and Agrobacterium rhizogenes are examples of plant pathogens that can transfer plasmid-encoded bacterial genes located on the T-DNA into plant cells in a manner dependent on the translocation of bacterial virulence (Vir) proteins. In some aspects, the method uses a disarmed Agrobacterium strain, such as but not limited to, AGL-1, EHA105, GV3101, LBA4404, LBA4404 THY-, and LBA4404 THY-TD, to transform explants with the first and second heterologous nucleotide sequences.

The methods, expression cassettes, and vectors described herein, can be used to generate or derive somatic embryos in dicot explants including those of the orders Ranunculales, Fabales, Cucurbitales, Malvales, Brassicales, Solanales, Asterales, and Apiales as exemplified herein. The methods, expression cassettes, and vectors described herein, can be used to generate or derive somatic embryos in Eudicot explants in the clades Campanulids, Lamids, Malvids, or Fabids, as well as in the families Papaveraceae, Fabaceae, Cucurbitaceae, Malvacaeae, Brassicaceae, Solanaceae, Asteraceae, or Apiaceae.

In some aspects, the explant is from cotton, Arabidopsis, poppy, carrot, alfalfa, soybean, flax, cassava, common bean, cowpea, tomato, potato, beet, grape, Eucalyptus, poplar, citrus, papaya, cacao, cucumber, apple, Capsicum, melon, or Brassica.

Suitable explant sources for somatic embryo induction, which can vary by dicot, include cotyledon, hypocotyl, epicotyl, leaves, stems and roots.

In some aspects, the dicot explant can be sourced from a transgenic plant. As used herein, “transgenic plant” and “transformed plant” refer to a plant that comprises within its genome a heterologous polynucleotide (e.g., transgene) that is, generally, stably integrated within the genome of the transgenic or transformed plant such that the polynucleotide is passed on to successive generations. The heterologous polynucleotide or transgene can be integrated into the genome alone or as part of a recombinant DNA construct. In this respect, re-transformation of transgenic dicot explants to generate somatic embryos is encompassed by the present disclosure.

In some aspects, the dicot explant can be sourced from non-transgenic plants.

In some aspects, the dicot explant is from a recalcitrant variety.

Somatic Embryo Formation in Cotton and Regeneration of Transgenic Cotton Plants

The present disclosure provides a method of inducing somatic embryo formation in cotton explants that includes transforming a cotton explant with a construct comprising a morphogenic gene cassette disclosed herein. Suitable cotton explant sources include cotyledon, hypocotyl, epicotyl, leaves, stems and roots. As shown in Examples herein, the disclosed methods not only improved the efficiency of somatic embryo formation, but also were surprisingly found to induce somatic embryo formation in multiple cotton lines (including elite cotton varieties) that are otherwise recalcitrant to somatic embryo induction. Accordingly, in one example of each of the methods of inducing somatic embryo formation disclosed herein for cotton, the cotton variety that is transformed can be a variety that prior to the disclosure of this application was recalcitrant to somatic embryo induction.

In one aspect, the method of inducing somatic embryo formation comprises transforming a cotton (e.g., a Pima or other variety) explant with a first nucleotide sequence encoding a BBM polypeptide (e.g., BBM1 or BBM2) and a second nucleotide sequence encoding a LEC polypeptide (e.g., LEC1 or LEC2). In one example, the method comprises transforming a cotton explant with a first nucleotide sequence encoding a BBM1 polypeptide having at least 90%, 91%, 92%, 93%, 94%, 95%, 96%, 97%, 98%, or 99% amino acid sequence identity to any of SEQ ID NOs: 5, 29, 31, 33, 35, 37, 39, 41, 43, 45, 47, 49, 51, 53, 55, 76, 78, or 80 and a second nucleotide sequence encoding a LEC2 polypeptide (e.g., SEQ ID NO:7). In another example, the method comprises transforming cotton (e.g., a Pima or other variety) explant with a first nucleotide sequence encoding a BBM1 polypeptide (e.g., SEQ ID NO:5) and a second nucleotide sequence encoding a LEC2 polypeptide having at least 90%, 91%, 92%, 93%, 94%, 95%, 96%, 97%, 98%, or 99% amino acid sequence identity to any of SEQ

ID NOs: 7, 13, 15, 17, 19, 21, 23, 25, or 27.

In a second aspect, the method of inducing somatic embryo formation comprises transforming a cotton (e.g., a Pima or other variety) explant with a first nucleotide sequence encoding a RKD4 polypeptide and a second nucleotide sequence encoding a LEC polypeptide (e.g., LEC1 or LEC2). In one example, the method comprises transforming a cotton (e.g., a Pima or other variety) explant with a first nucleotide sequence encoding a RKD4 polypeptide having at least 90%, 91%, 92%, 93%, 94%, 95%, 96%, 97%, 98%, or 99% amino acid sequence identity to SEQ ID NO:9 and a second nucleotide sequence encoding a LEC2 polypeptide (e.g., SEQ ID NO:7). In another example, the method comprises transforming a cotton (e.g., a Pima or other variety) explant with a first nucleotide sequence encoding the RKD4 polypeptide and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a third aspect, the method of inducing somatic embryo formation comprises transforming a cotton (e.g., a Pima or other variety) explant with a first nucleotide sequence encoding a CCAAT-Binding Factor 1A Activation Domain (CBF1A) coupled to BBM polypeptide and a second nucleotide sequence encoding a LEC polypeptide (e.g., LEC1 or LEC2). A nucleotide sequence encoding a CBF1A disclosed herein can have at least 90%, 91%, 92%, 93%, 94%, 95%, 96%, 97%, 98%, or 99% identity to SEQ ID NO:70, or it can encode a CBF1A polypeptide having at least 90%, 91%, 92%, 93%, 94%, 95%, 96%, 97%, 98%, or 99% identity to SEQ ID NO:71.

In a fourth aspect, the method of inducing somatic embryo formation comprises transforming a cotton (e.g., a Pima or other variety) explant with a first nucleotide sequence encoding BBM and a second nucleotide sequence encoding a CBF1A coupled to a LEC polypeptide (e.g., LEC1 or LEC2). In a further variation both the first nucleotide sequence encoding a BBM and the second nucleotide sequence encoding a LEC polypeptide are each coupled to a sequence encoding CBF1A.

In a fifth aspect, the method of inducing somatic embryo formation comprises transforming a cotton (e.g., a Pima or other variety) explant with a nucleotide sequence encoding a CBF1A coupled to a truncated version of a BBM polypeptide, where the truncated BBM comprises the BBM AP2 Domain and C-terminus, but lacks BBM N-terminus.

In a sixth aspect, the method of inducing somatic embryo formation comprises transforming a cotton (e.g., a Pima or other variety) explant with a first nucleotide sequence encoding a Glucocorticoid Receptor (GR) coupled to BBM polypeptide and a second nucleotide sequence encoding a LEC polypeptide (e.g., LEC1 or LEC2). In a seventh aspect, the method of inducing somatic embryo formation comprises transforming a cotton (e.g., a Pima or other variety) explant with a first nucleotide sequence encoding BBM and a second nucleotide sequence encoding a GR coupled to a LEC polypeptide (e.g., LEC1 or LEC2). In a further variation both the first nucleotide sequence encoding a BBM and the second nucleotide sequence encoding a LEC polypeptide are each coupled to sequence encoding GR. A nucleotide sequence encoding a GR domain disclosed herein can have at least 90%, 91%, 92%, 93%, 94%, 95%, 96%, 97%, 98%, or 99% identity to SEQ ID NO:73, or it can encode a GR polypeptide having at least 90%, 91%, 92%, 93%, 94%, 95%, 96%, 97%, 98%, or 99% identity to SEQ ID NO:74.

In an eighth aspect, the method of inducing somatic embryo formation comprises transforming a cotton (e.g., a Pima or other variety) explant with a first nucleotide sequence encoding a BBM polypeptide (e.g., BBM1 or BBM2) and a second nucleotide sequence encoding a FUSCA3 (FUS3) polypeptide. The FUS3 polypeptide can have at least 90%, 91%, 92%, 93%, 94%, 95%, 96%, 97%, 98%, or 99% amino acid sequence identity to SEQ ID NO: 57.

Each of the foregoing aspects of inducing somatic embryo formation in cotton can further comprise regenerating a transgenic cotton plant from the somatic embryo. Generally, regenerating a plant from the transformed somatic embryo tissue can include transferring the embryo to embryo germination medium and/or rooting and elongation medium to regenerate a transgenic cotton plant. In preferred embodiments, the first and second nucleotide sequences of the morphogenic gene cassette used to induce somatic embryo formation are inactivated prior to or during the regeneration of a transgenic cotton plant. Such inactivation can be done by any suitable means, e.g., by inactivating or suppressing the expression of the two encoded heterologous morphogenic proteins or by excising the heterologous nucleotide sequences encoding the morphogenic proteins. Thus, for example, after somatic embryo induction, the first and second heterologous nucleotide sequence of the morphogenic gene cassette can be excised prior to the completing the process of regenerating a transgenic plant.

Techniques and media useful for cotton transformation are described in U.S. Pat. Nos. 5,846,797, 5,159,135, 5,004,863, and 6,624,344, and also in protocols published electronically by IP.com using permanent publication identifiers IPCOM000033402D, IPCOM000033403D, IPCOM000033404D, which can be retrieved using the online database search portal hosted by IP.com, as well as in the Examples provided herein.

Somatic Embryo Formation in Alfalfa and Regeneration of Transgenic Alfalfa Plants

The present disclosure provides a method of inducing somatic embryo formation in alfalfa explants that includes transforming an alfalfa explant with a construct comprising a morphogenic gene cassette disclosure herein. Suitable alfalfa explant sources include leaves, stems, and roots.

In one aspect, the method of inducing somatic embryo formation comprises transforming an alfalfa explant with a first nucleotide sequence encoding a BBM polypeptide (e.g., BBM1 or BBM2) and a second nucleotide sequence encoding a LEC polypeptide (e.g., LEC1 or LEC2). In one example, the method comprises transforming an alfalfa explant with a first nucleotide sequence encoding a BBM1 polypeptide having at least 90%, 91%, 92%, 93%, 94%, 95%, 96%, 97%, 98%, or 99% amino acid sequence identity to any of SEQ ID NOs: 5, 29, 31, 33, 35, 37, 39, 41, 43, 45, 47, 49, 51, 53, 55, 76, 78, or 80 and a second nucleotide sequence encoding a LEC2 polypeptide (e.g., SEQ ID NO:7). In another example, the method comprises transforming an alfalfa explant with a first nucleotide sequence encoding a BBM1 polypeptide (e.g., SEQ ID NO:5) and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a second aspect, the method of inducing somatic embryo formation comprises transforming an alfalfa explant with a first nucleotide sequence encoding a RKD4 polypeptide and a second nucleotide sequence encoding a LEC polypeptide (e.g., LEC1 or LEC2). A nucleotide sequence encoding a RKD4 disclosed herein can have at least 90%, 91%, 92%, 93%, 94%, 95%, 96%, 97%, 98%, or 99% identity to SEQ ID NO:8, or encoding a RKD4 polypeptide having at least 90%, 91%, 92%, 93%, 94%, 95%, 96%, 97%, 98%, or 99% amino acid sequence identity to SEQ ID NO:9. In one example, the method comprises transforming an alfalfa explant with a first nucleotide sequence having a RKD4 polypeptide and a second nucleotide sequence encoding a LEC2 polypeptide (e.g., SEQ ID NO:7). In another example, the method comprises transforming an alfalfa explant with a first nucleotide sequence encoding the RKD4 polypeptide and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a third aspect, the method of inducing somatic embryo formation comprises transforming an alfalfa explant with a first nucleotide sequence encoding a CCAAT-Binding Factor 1A Activation Domain (CBF1A) coupled to BBM polypeptide and a second nucleotide sequence encoding a LEC polypeptide (e.g., LEC1 or LEC2). A nucleotide sequence encoding a CBF1A disclosed herein can have at least 90%, 91%, 92%, 93%, 94%, 95%, 96%, 97%, 98%, or 99% identity to SEQ ID NO:70, or it can encode a CBF1A polypeptide having at least 90%, 91%, 92%, 93%, 94%, 95%, 96%, 97%, 98%, or 99% identity to SEQ ID NO:71.

In a fourth aspect, the method of inducing somatic embryo formation comprises transforming an alfalfa explant with a first nucleotide sequence encoding BBM and a second nucleotide sequence encoding a CBF1A coupled to a LEC polypeptide (e.g., LEC1 or LEC2).

In a fifth aspect, the method of inducing somatic embryo formation comprises transforming an alfalfa explant with a first nucleotide sequence encoding BBM AP2 domain and a second nucleotide sequence encoding a LEC polypeptide (e.g., LEC1 or LEC2). In a variation of this aspect, the first nucleotide sequence can encode a BBM AP2 domain that is coupled to CBF1A and the second nucleotide sequence encodes a LEC polypeptide (e.g., LEC1 or LEC2). A nucleotide sequence encoding a BBM AP2 domain disclosed herein can have at least 90%, 91%, 92%, 93%, 94%, 95%, 96%, 97%, 98%, or 99% identity to SEQ ID NO:68, or it can encode a BBM AP2 domain polypeptide having at least 90%, 91%, 92%, 93%, 94%, 95%, 96%, 97%, 98%, or 99% identity to SEQ ID NO:69.

In a sixth aspect, the method of inducing somatic embryo formation comprises transforming an alfalfa explant with a first nucleotide sequence encoding a Glucocorticoid Receptor (GR) coupled to BBM polypeptide and a second nucleotide sequence encoding a LEC polypeptide (e.g., LEC1 or LEC2). In a variation of this aspect, the method of inducing somatic embryo formation comprises transforming an alfalfa explant with a first nucleotide sequence encoding BBM and a second nucleotide sequence encoding a GR coupled to a LEC polypeptide (e.g., LEC1 or LEC2). A nucleotide sequence encoding a GR domain disclosed herein can have at least 90%, 91%, 92%, 93%, 94%, 95%, 96%, 97%, 98%, or 99% identity to SEQ ID NO:73, or it can encode a GR polypeptide having at least 90%, 91%, 92%, 93%, 94%, 95%, 96%, 97%, 98%, or 99% identity to SEQ ID NO:74.

In a seventh aspect, the method of inducing somatic embryo formation comprises transforming an alfalfa explant with a first nucleotide sequence encoding a BBM polypeptide (e.g., BBM1 or BBM2) and a second nucleotide sequence encoding a FUS3 polypeptide. The FUS3 polypeptide can have at least 90%, 91%, 92%, 93%, 94%, 95%, 96%, 97%, 98%, or 99% amino acid sequence identity to SEQ ID NO:57.

In an eighth aspect, the method of inducing somatic embryo formation comprises transforming an alfalfa explant with a first nucleotide sequence encoding a BBM polypeptide (e.g., BBM1 or BBM2) and a second nucleotide sequence encoding an ABI3 polypeptide. The ABI3 polypeptide can have at least 90%, 91%, 92%, 93%, 94%, 95%, 96%, 97%, 98%, or 99% amino acid sequence identity to SEQ ID NO:64.

Each of the foregoing aspects of inducing somatic embryo formation in alfalfa can further comprise regenerating a transgenic alfalfa plant from the somatic embryo. Generally, regenerating a plant from the transformed somatic embryo tissue can include transferring the embryo to embryo germination medium and/or rooting and elongation medium to regenerate a transgenic alfalfa plant. In preferred embodiments, the first and second nucleotide sequences of the morphogenic gene cassette used to induce somatic embryo formation are inactivated prior to or during the regeneration of a transgenic alfalfa plant. Such inactivation can be done by any suitable means, e.g., by inactivating or suppressing the expression of the two encoded heterologous morphogenic proteins or by excising the heterologous nucleotide sequences encoding the morphogenic proteins. Thus, for example, after somatic embryo induction, the first and second heterologous nucleotide sequence of the morphogenic gene cassette can be excised prior to the completing the process of regenerating a transgenic plant.

Techniques and media useful for alfalfa transformation are described in U.S. Pat. Nos. 4,801,545, 6,566,137; 8,124,848; and U.S. Pat. No. 9,771,597; Samac et al., (Samac D. A., Austin-Phillips S. (2006) Alfalfa (Medicago sativa L.). In: Wang K. (eds) Agrobacterium Protocols. Methods in Molecular Biology, vol 343), as well as in the Examples provided herein.

Somatic Embryo Formation in Canola and Regeneration of Transgenic Canola Plants

The present disclosure provides a method of inducing somatic embryo formation in Brassica napus or canola explants that includes transforming a canola explant with a construct comprising a morphogenic gene cassette disclosure herein. Suitable canola explant sources include cotyledon, hypocotyl, epicotyl, leaves, stems and roots. As used herein, the term “canola” refers to any Brassica napus, including spring or winter (vernalizing) varieties.

In one aspect, the method of inducing somatic embryo formation comprises transforming a canola explant with a first nucleotide sequence encoding a BBM polypeptide (e.g., BBM1 or BBM2) and a second nucleotide sequence encoding a LEC polypeptide (e.g., LEC1 or LEC2). In one example, the method comprises transforming a canola explant with a first nucleotide sequence encoding a BBM1 polypeptide having at least 90%, 91%, 92%, 93%, 94%, 95%, 96%, 97%, 98%, or 99% amino acid sequence identity to any of SEQ ID NOs: 5, 29, 31, 33, 35, 37, 39, 41, 43, 45, 47, 49, 51, 53, 55, 76, 78, or 80 and a second nucleotide sequence encoding a LEC2 polypeptide (e.g., SEQ ID NO:7). In another example, the method comprises transforming a canola explant with a first nucleotide sequence encoding a BBM1 polypeptide (e.g., SEQ ID NO:5) and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a second aspect, the method of inducing somatic embryo formation comprises transforming a canola explant with a first nucleotide sequence encoding a RKD4 polypeptide and a second nucleotide sequence encoding a LEC polypeptide (e.g., LEC1 or LEC2). In one example, the method comprises transforming a canola explant with a first nucleotide sequence encoding a RKD4 polypeptide having at least 90%, 91%, 92%, 93%, 94%, 95%, 96%, 97%, 98%, or 99% amino acid sequence identity to SEQ ID NO:9 and a second nucleotide sequence encoding a LEC2 polypeptide (e.g., SEQ ID NO:7). In another example, the method comprises transforming a canola explant with a first nucleotide sequence encoding the RKD4 polypeptide and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a third aspect, the method of inducing somatic embryo formation comprises transforming a canola explant with a first nucleotide sequence encoding a CCAAT-Binding Factor 1A Activation Domain (CBF1A) coupled to BBM polypeptide and a second nucleotide sequence encoding a LEC polypeptide (e.g., LEC1 or LEC2). A nucleotide sequence encoding a CBF1A disclosed herein can have at least 90%, 91%, 92%, 93%, 94%, 95%, 96%, 97%, 98%, or 99% identity to SEQ ID NO:70, or it can encode a CBF1A polypeptide having at least 90%, 91%, 92%, 93%, 94%, 95%, 96%, 97%, 98%, or 99% identity to SEQ ID NO:71.

In a fourth aspect, the method of inducing somatic embryo formation comprises transforming a canola explant with a first nucleotide sequence encoding BBM and a second nucleotide sequence encoding a CBF1A coupled to a LEC polypeptide (e.g., LEC1 or LEC2).

In a fifth aspect, the method of inducing somatic embryo formation comprises transforming a canola explant with a first nucleotide sequence encoding BBM AP2 domain and a second nucleotide sequence encoding a LEC polypeptide (e.g., LEC1 or LEC2). In a variation of this aspect, the first nucleotide sequence can encode a BBM AP2 domain that is coupled to CBF1A and the second nucleotide sequence encodes a LEC polypeptide (e.g., LEC1 or LEC2). A nucleotide sequence encoding a BBM AP2 domain disclosed herein can have at least 90%, 91%, 92%, 93%, 94%, 95%, 96%, 97%, 98%, or 99% identity to SEQ ID NO:68, or it can encode a BBM AP2 domain polypeptide having at least 90%, 91%, 92%, 93%, 94%, 95%, 96%, 97%, 98%, or 99% identity to SEQ ID NO:69.

In a sixth aspect, the method of inducing somatic embryo formation comprises transforming a canola explant with a first nucleotide sequence encoding a Glucocorticoid Receptor (GR) coupled to BBM polypeptide and a second nucleotide sequence encoding a LEC polypeptide (e.g., LEC1 or LEC2). In a variation of this aspect, the method of inducing somatic embryo formation comprises transforming a canola explant with a first nucleotide sequence encoding BBM and a second nucleotide sequence encoding a GR coupled to a LEC polypeptide (e.g., LEC1 or LEC2). A nucleotide sequence encoding a GR domain disclosed herein can have at least 90%, 91%, 92%, 93%, 94%, 95%, 96%, 97%, 98%, or 99% identity to SEQ ID NO:73, or it can encode a GR polypeptide having at least 90%, 91%, 92%, 93%, 94%, 95%, 96%, 97%, 98%, or 99% identity to SEQ ID NO:74.

In a seventh aspect, the method of inducing somatic embryo formation comprises transforming a canola explant with a first nucleotide sequence encoding a BBM polypeptide (e.g., BBM1 or BBM2) and a second nucleotide sequence encoding a FUS3 polypeptide. The FUS3 polypeptide can have at least 90%, 91%, 92%, 93%, 94%, 95%, 96%, 97%, 98%, or 99% amino acid sequence identity to SEQ ID NO:57.

In an eighth aspect, the method of inducing somatic embryo formation comprises transforming a canola explant with a first nucleotide sequence encoding a BBM polypeptide (e.g., BBM1 or BBM2) and a second nucleotide sequence encoding an ABI3 polypeptide. The ABI3 polypeptide can have at least 90%, 91%, 92%, 93%, 94%, 95%, 96%, 97%, 98%, or 99% amino acid sequence identity to SEQ ID NO:64.

Each of the foregoing aspects of inducing somatic embryo formation in canola can further comprise regenerating a transgenic canola plant from the somatic embryo. Generally, regenerating a plant from the transformed somatic embryo tissue can include transferring the embryo to embryo germination medium and/or rooting and elongation medium to regenerate a transgenic canola plant. In preferred embodiments, the first and second nucleotide sequences of the morphogenic gene cassette used to induce somatic embryo formation are inactivated prior to or during the regeneration of a transgenic canola plant. Such inactivation can be done by any suitable means, e.g., by inactivating or suppressing the expression of the two encoded heterologous morphogenic proteins or by excising the heterologous nucleotide sequences encoding the morphogenic proteins. Thus, for example, after somatic embryo induction, the first and second heterologous nucleotide sequence of the morphogenic gene cassette can be excised prior to the completing the process of regenerating a transgenic plant.

Techniques and media useful for canola transformation are described in U.S. Pat. No. 5,750,871 and Chu et al. (2020) Genotype-independent transformation and genome editing of Brassica napus using a novel explant material. Frontiers in Plant Sciences 11 (579524): 1-11, as well as in the Examples provided herein.

### Somatic Embryo Formation in California Poppy, Cucumber, Carrot, or Tomato and Regeneration of Transgenic California Poppy, Cucumber, Carrot, or Tomato Plants

The present disclosure provides a method of inducing somatic embryo formation in California poppy, cucumber, carrot, or tomato explants that includes transforming a California poppy explant, a cucumber explant, a carrot explant, or a tomato explant with a construct comprising a morphogenic gene cassette disclosed herein. Suitable California poppy, cucumber, carrot, or tomato explant sources include cotyledon, hypocotyl, epicotyl, leaves, stems and roots.

In one aspect, the method of inducing somatic embryo formation comprises transforming an explant (i.e., a California poppy explant, a cucumber explant, a carrot explant, or a tomato explant) with a first nucleotide sequence encoding a BBM polypeptide (e.g., BBM1 or BBM2) and a second nucleotide sequence encoding a LEC polypeptide (e.g., LEC1 or LEC2). In one example, the method comprises transforming an explant with a first nucleotide sequence encoding a BBM1 polypeptide having at least 90%, 91%, 92%, 93%, 94%, 95%, 96%, 97%, 98%, or 99% amino acid sequence identity to any of SEQ ID NOs: 5, 29, 31, 33, 35, 37, 39, 41, 43, 45, 47, 49, 51, 53, 55, 76, 78, or 80 and a second nucleotide sequence encoding a LEC2 polypeptide (e.g., SEQ ID NO:7). In another example, the method comprises transforming an explant with a first nucleotide sequence encoding a BBM1 polypeptide (e.g., SEQ ID NO:5) and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a second aspect, the method of inducing somatic embryo formation comprises transforming an explant (i.e., a California poppy explant, a cucumber explant, a carrot explant, or a tomato explant) with a first nucleotide sequence encoding a RKD4 polypeptide and a second nucleotide sequence encoding a LEC polypeptide (e.g., LEC1 or LEC2). In one example, the method comprises transforming the explant with a first nucleotide sequence encoding a RKD4 polypeptide having at least 90%, 91%, 92%, 93%, 94%, 95%, 96%, 97%, 98%, or 99% amino acid sequence identity to SEQ ID NO:9 and a second nucleotide sequence encoding a LEC2 polypeptide (e.g., SEQ ID NO:7). In another example, the method comprises transforming the explant with a first nucleotide sequence encoding the RKD4 polypeptide and a second nucleotide sequence encoding a LEC2 polypeptide having at least 90%, 91%, 92%, 93%, 94%, 95%, 96%, 97%, 98%, or 99% amino acid sequence identity to any of SEQ ID NOs:

7, 13, 15, 17, 19, 21, 23, 25, or 27.

In a third aspect, the method of inducing somatic embryo formation comprises transforming an explant (i.e., a California poppy explant, a cucumber explant, a carrot explant, or a tomato explant) with a first nucleotide sequence encoding a CCAAT-Binding Factor 1A Activation Domain (CBF1A) coupled to BBM polypeptide and a second nucleotide sequence encoding a LEC polypeptide (e.g., LEC1 or LEC2). A nucleotide sequence encoding a CBF1A disclosed herein can have at least 90%, 91%, 92%, 93%, 94%, 95%, 96%, 97%, 98%, or 99% identity to SEQ ID NO:70, or it can encode a CBF1A polypeptide having at least 90%, 91%, 92%, 93%, 94%, 95%, 96%, 97%, 98%, or 99% identity to SEQ ID NO:71.

In a fourth aspect, the method of inducing somatic embryo formation comprises transforming an explant (i.e., a California poppy explant, a cucumber explant, a carrot explant, or a tomato explant) with a first nucleotide sequence encoding BBM and a second nucleotide sequence encoding a CBF1A coupled to a LEC polypeptide (e.g., LEC1 or LEC2).

In a fifth aspect, the method of inducing somatic embryo formation comprises transforming an explant (i.e., a California poppy explant, a cucumber explant, a carrot explant, or a tomato explant) with a first nucleotide sequence encoding BBM AP2 domain and a second nucleotide sequence encoding a LEC polypeptide (e.g., LEC1 or LEC2). In a variation of this aspect, the first nucleotide sequence can encode a BBM AP2 domain that is coupled to CBF1A and the second nucleotide sequence encodes a LEC polypeptide (e.g., LEC1 or LEC2). A nucleotide sequence encoding a BBM AP2 domain disclosed herein can have at least 90%, 91%, 92%, 93%, 94%, 95%, 96%, 97%, 98%, or 99% identity to SEQ ID NO:68, or it can encode a BBM AP2 domain polypeptide having at least 90%, 91%, 92%, 93%, 94%, 95%, 96%, 97%, 98%, or 99% identity to SEQ ID NO:69.

In a sixth aspect, the method of inducing somatic embryo formation comprises transforming an explant (i.e., a California poppy explant, a cucumber explant, a carrot explant, or a tomato explant) with a first nucleotide sequence encoding a Glucocorticoid Receptor (GR) coupled to BBM polypeptide and a second nucleotide sequence encoding a LEC polypeptide (e.g., LEC1 or LEC2). In a variation of this aspect, the method of inducing somatic embryo formation comprises transforming an explant (i.e., a California poppy explant, a cucumber explant, a carrot explant, or a tomato explant) with a first nucleotide sequence encoding BBM and a second nucleotide sequence encoding a GR coupled to a LEC polypeptide (e.g., LEC1 or LEC2). A nucleotide sequence encoding a GR domain disclosed herein can have at least 90%, 91%, 92%, 93%, 94%, 95%, 96%, 97%, 98%, or 99% identity to SEQ ID NO:73, or it can encode a GR polypeptide having at least 90%, 91%, 92%, 93%, 94%, 95%, 96%, 97%, 98%, or 99% identity to SEQ ID NO:74.

In a seventh aspect, the method of inducing somatic embryo formation comprises transforming an explant (i.e., a California poppy explant, a cucumber explant, a carrot explant, or a tomato explant) with a first nucleotide sequence encoding a BBM polypeptide (e.g., BBM1 or BBM2) and a second nucleotide sequence encoding a FUS3 polypeptide. The FUS3 polypeptide can have at least 90%, 91%, 92%, 93%, 94%, 95%, 96%, 97%, 98%, or 99% amino acid sequence identity to SEQ ID NO:57.

In an eighth aspect, the method of inducing somatic embryo formation comprises transforming an explant (i.e., a California poppy explant, a cucumber explant, a carrot explant, or a tomato explant) with a first nucleotide sequence encoding a BBM polypeptide (e.g., BBM1 or BBM2) and a second nucleotide sequence encoding an ABI3 polypeptide. The ABI3 polypeptide can have at least 90%, 91%, 92%, 93%, 94%, 95%, 96%, 97%, 98%, or 99% amino acid sequence identity to SEQ ID NO:64.

Each of the foregoing aspects of inducing somatic embryo formation in California poppy, cucumber, carrot, or tomato can further comprise regenerating a transgenic plant from the somatic embryo. Generally, regenerating a plant from the transformed somatic embryo tissue can include transferring the embryo to embryo germination medium and/or rooting and elongation medium to regenerate a transgenic California poppy, cucumber, carrot, or tomato plant. In preferred embodiments, the first and second nucleotide sequences of the morphogenic gene cassette used to induce somatic embryo formation are inactivated prior to or during the regeneration of a transgenic plant. Such inactivation can be done by any suitable means, e.g., by inactivating or suppressing the expression of the two encoded heterologous morphogenic proteins or by excising the heterologous nucleotide sequences encoding the morphogenic proteins. Thus, for example, after somatic embryo induction, the first and second heterologous nucleotide sequence of the morphogenic gene cassette can be excised prior to the completing the process of regenerating a transgenic plant.

Techniques and media useful for the transformation of California poppy, cucumber, carrot, and tomato are described in Park et al., (Park, S U., Facchini, P. Agrobacterium-mediated genetic transformation of California poppy, Eschscholzia californica Cham., via somatic embryogenesis. Plant Cell Reports 19, 1006-1012 (2000)); Techniques and media useful for transformation are described in Burza et al., (Burza W., Zuzga S., Yin Z., Malepszy S. (2006) Cucumber (Cucumis sativus L.). Wang K. (eds) Agrobacterium Protocols. Methods in Molecular Biology, vol 343), Hardegger et al., (Hardegger, M., Sturm, A. Transformation and regeneration of carrot (Daucus carota L.). Molecular Breeding 4, 119-127 (1998)), Sharma et al., (Sharma, M. K., Solanke, A. U., Jani, D. et al. A simple and efficient Agrobacterium-mediated procedure for transformation of tomato. J Biosci 34, 423-433 (2009)), as well as in the Examples provided herein.

Somatic Embryo Formation in Sunflower and Regeneration of Transgenic Sunflower. The present disclosure provides a method of inducing somatic embryo formation in sunflower (Helianthus annuus) explants that includes transforming a sunflower explant with a construct comprising a morphogenic gene cassette disclosure herein. Suitable sunflower explant sources include cotyledon, hypocotyl, epicotyl, leaves, stems and roots.

In one aspect, the method of inducing somatic embryo formation comprises transforming sunflower explant with a first nucleotide sequence encoding a BBM polypeptide (e.g., BBM1 or BBM2) and a second nucleotide sequence encoding a LEC polypeptide (e.g., LEC1 or LEC2). In one example, the method comprises transforming sunflower explant with a first nucleotide sequence encoding a BBM1 polypeptide having at least 90%, 91%, 92%, 93%, 94%, 95%, 96%, 97%, 98%, or 99% amino acid sequence identity to any of SEQ ID NOs: 5, 29, 31, 33, 35, 37, 39, 41, 43, 45, 47, 49, 51, 53, 55, 76, 78, or 80 and a second nucleotide sequence encoding a LEC2 polypeptide (e.g., SEQ ID NO:7). In another example, the method comprises transforming sunflower explant with a first nucleotide sequence encoding a BBM1 polypeptide (e.g., SEQ ID NO:5) and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a second aspect, the method of inducing somatic embryo formation comprises transforming sunflower explant with a first nucleotide sequence encoding a RKD4 polypeptide and a second nucleotide sequence encoding a LEC polypeptide (e.g., LEC1 or LEC2). In one example, the method comprises transforming a sunflower explant with a first nucleotide sequence encoding a RKD4 polypeptide having at least 90%, 91%, 92%, 93%, 94%, 95%, 96%, 97%, 98%, or 99% amino acid sequence identity to SEQ ID NO:9 and a second nucleotide sequence encoding a LEC2 polypeptide (e.g., SEQ ID NO:7). In another example, the method comprises transforming sunflower explant with a first nucleotide sequence encoding the RKD4 polypeptide and a second nucleotide sequence encoding a LEC2 polypeptide having at least 90%, 91%, 92%, 93%, 94%, 95%, 96%, 97%, 98%, or 99% amino acid sequence identity to any of SEQ ID NOs: 7, 13, 15, 17, 19, 21, 23, 25, or 27.

In a third aspect, the method of inducing somatic embryo formation comprises transforming sunflower explant with a first nucleotide sequence encoding a CCAAT-Binding Factor 1A Activation Domain (CBF1A) coupled to BBM polypeptide and a second nucleotide sequence encoding a LEC polypeptide (e.g., LEC1 or LEC2). A nucleotide sequence encoding a CBF1A disclosed herein can have at least 90%, 91%, 92%, 93%, 94%, 95%, 96%, 97%, 98%, or 99% identity to SEQ ID NO:70, or it can encode a CBF1A polypeptide having at least 90%, 91%, 92%, 93%, 94%, 95%, 96%, 97%, 98%, or 99% identity to SEQ ID NO:71.

In a fourth aspect, the method of inducing somatic embryo formation comprises transforming sunflower explant with a first nucleotide sequence encoding BBM and a second nucleotide sequence encoding a CBF1A coupled to a LEC polypeptide (e.g., LEC1 or LEC2).

In a fifth aspect, the method of inducing somatic embryo formation comprises transforming sunflower explant with a first nucleotide sequence encoding BBM AP2 domain and a second nucleotide sequence encoding a LEC polypeptide (e.g., LEC1 or LEC2). In a variation of this aspect, the first nucleotide sequence can encode a BBM AP2 domain that is coupled to CBF1A and the second nucleotide sequence encodes a LEC polypeptide (e.g., LEC1 or LEC2). A nucleotide sequence encoding a BBM AP2 domain disclosed herein can have at least 90%, 91%, 92%, 93%, 94%, 95%, 96%, 97%, 98%, or 99% identity to SEQ ID NO:68, or it can encode a BBM AP2 domain polypeptide having at least 90%, 91%, 92%, 93%, 94%, 95%, 96%, 97%, 98%, or 99% identity to SEQ ID NO:69.

In a sixth aspect, the method of inducing somatic embryo formation comprises transforming sunflower explant with a first nucleotide sequence encoding a Glucocorticoid Receptor (GR) coupled to BBM polypeptide and a second nucleotide sequence encoding a LEC polypeptide (e.g., LEC1 or LEC2). In a variation of this aspect, the method of inducing somatic embryo formation comprises transforming sunflower explant with a first nucleotide sequence encoding BBM and a second nucleotide sequence encoding a GR coupled to a LEC polypeptide (e.g., LEC1 or LEC2). A nucleotide sequence encoding a GR domain disclosed herein can have at least 90%, 91%, 92%, 93%, 94%, 95%, 96%, 97%, 98%, or 99% identity to SEQ ID NO:73, or it can encode a GR polypeptide having at least 90%, 91%, 92%, 93%, 94%, 95%, 96%, 97%, 98%, or 99% identity to SEQ ID NO:74.

In a seventh aspect, the method of inducing somatic embryo formation comprises transforming sunflower explant with a first nucleotide sequence encoding a BBM polypeptide (e.g., BBM1 or BBM2) and a second nucleotide sequence encoding a FUS3 polypeptide. The FUS3 polypeptide can have at least 90%, 91%, 92%, 93%, 94%, 95%, 96%, 97%, 98%, or 99% amino acid sequence identity to SEQ ID NO:57.

In an eighth aspect, the method of inducing somatic embryo formation comprises transforming sunflower explant with a first nucleotide sequence encoding a BBM polypeptide (e.g., BBM1 or BBM2) and a second nucleotide sequence encoding an ABI3 polypeptide. The ABI3 polypeptide can have at least 90%, 91%, 92%, 93%, 94%, 95%, 96%, 97%, 98%, or 99% amino acid sequence identity to SEQ ID NO:64.

Each of the foregoing aspects of inducing somatic embryo formation in sunflower can further comprise regenerating a transgenic sunflower plant from the somatic embryo. Generally, regenerating a plant from the transformed somatic embryo tissue can include transferring the embryo to embryo germination medium and/or rooting and elongation medium to regenerate a transgenic sunflower plant. In preferred embodiments, the first and second nucleotide sequences of the morphogenic gene cassette used to induce somatic embryo formation are inactivated prior to or during the regeneration of a transgenic canola plant. Such inactivation can be done by any suitable means, e.g., by inactivating or suppressing the expression of the two encoded heterologous morphogenic proteins or by excising the heterologous nucleotide sequences encoding the morphogenic proteins. Thus, for example, after somatic embryo induction, the first and second heterologous nucleotide sequence of the morphogenic gene cassette can be excised prior to the completing the process of regenerating a transgenic plant.

Techniques and media useful for sunflower transformation are described, e.g., in International Application Publication WO 2021/195058, Sankara et al. “Agrobacterium-Mediated Transformation of Sunflower (Helianthus Annuus L.) A Simple Protocol”, Annals of Botany, Academic Press (London, GB), Vol. 83 (4): 347-354 (1999) and Schrammeijer et al., Plant Cell Reports, 9:55-60 (1990), as well as in the Examples herein.

The following are examples of specific examples of some aspects of the invention. The examples are offered for illustrative purposes only, and they are not intended to limit the scope of the invention in any way. Efforts have been made to ensure accuracy with respect to numbers used (e.g., amounts, temperatures, etc.), but some experimental error and deviation should, of course, be allowed for.

### EXAMPLES

Plasmids used for transformation in the Examples described herein are described in Table 1, which shows T-DNA elements and corresponding sequence listing identifier. Media compositions are described in Table 2. Unless otherwise indicated, seeds of different species were surface sterilized with 70% ethanol for 2 min and 20% bleach solution containing 2-4 drops of Tween 20 for 10 min. In some cases seeds were surface sterilized using a solution of 3% of hydrogen peroxide containing 2-4 drops of Tween 20 for 30 minutes. Seeds were rinsed thoroughly with sterile water and germinated on semi-solid media. Unless otherwise indicated infections were carried using Agrobacterium suspensions in 20A medium (Table 2) adjusted to an OD of 1.0 at 600 nm; and explants were incubated with Agrobacterium suspension supplemented with 100 uM acetosyringone in 100×25 mm petri dishes for 10 min. The Agrobacterium suspension was poured off and the explants co-cultivated in a growth chamber under low light (12 μmol m⁻² s⁻¹) for 2-4 days. Explants were then transferred onto selection medium for 3 weeks. Explants were sub-cultured for an additional 3 weeks for somatic embryo induction, if required. Morphogenic genes were excised by heat-shocking the somatic embryos twice, 24 hours apart, at 45° C., 70% RH for 2 h. Heat-shocked embryos were transferred to regeneration medium and subcultured every 3 weeks until rooted plants were obtained.

TABLE 2

| Media ID | Ingredients |
|---|---|
| 90 | MS salt (2.165 g/l), 20 mg/l sucrose, 8 g/l agar |
| 20A | MS salt, 0.1 mg/l NAA (naphthaleneacetic acid), 1 mg/l BAP (6- |
| | benzylaminopurine), 0.01 mg/l gibberellic acid, 0.05 mg/l thymidine, MS |
| | Vitamins (0.5 mg/l nicotinic acid, 0.1 mg/l thiamine•HCl, 0.5 mg/l |
| | pyridoxine•HCl, 2 mg/l glycine) |
| 70AY | MS salt, 0.3 mg/l NAA, 0.5 mg/l BAP, 0.03 mg/l gibberellic acid, 5 mg/l |
| | spectinomycin, 20 g/l sucrose, MS Vitamins |
| 90A | MS salt, 0.5 mg/l IBA (indole butyric acid), 10 mg/l sucrose |
| 199M | 4.44 g/l MS Modified Basal with Gamborg Vitamins, 20 g/l glucose, 1.5 g/l |
| | activated charcoal, 8 g/l TC Agar, 200 mg/l Timentin |
| 14580C | MS salt + 3 g/l gellan gum gelling agent (Gelrite ™) |
| 14580AJ | MS salt + 50 mg/l thymidine and 0.2 mM acetosyringine |
| 14580AM | MS salt + 25 mg/l spectinomycin + 200 mg/l Tinmentin + 1 g/l Gelrite ™ |
| 14580K | MS salt + 25 mg/l spectinomycin + 200 mg/l Timentin + 3 g/l Gelrite ™ |
| 14580P | MS salt + 200 mg/l Timentin + 1 g/l Gelrite ™ |
| 14582F | 0.5 g/l Potassium Nitrate, 0.24 g/l Ammonium nitrate, 0.5 g/l magnesium sulphate, |
| | 0.03 g/l potassium phosphate monobasic, 0.2 g/l CaCl2, 5 mg/l FeNa-EDTA, |
| | 0.002 g/l Boric acid, 0.5 mg/l manganase sulphate, 2.5 mg/l zinc sulfate, 0.25 mg/l |
| | potassium iodide, 0.22 mg/l sodium molybdate, 0.0075 mg/l cupric sulfate, 1 mM |
| | cobalt chloride, 0.5 mg/l nicotinic acid, 0.8 mg/l pyridoxine HCl, 1.35 mg/l |
| | thiamine HCl, 20 g/l Glucose, 2 g/l gelling agent 2 g/l gellan gum gelling agent |
| | (Gelrite ™) |
| 17862 | 4.43 g/l MS Basal medium with vitamins, 30 g/l maltose, 30 g/l sorbitol, 1.5 g/l |
| | activated charcoal, 15 g/l TC Agar, 3 g/l Gelrite ™, 200 mg/l Timentin |
| 17202B | Schenk & Hildebrandt Basal salt mix 3.2 g/l, 0.1 g/l myo-inositol, 5 ml/l MS |
| | Vitamin, 30 g/l sucrose, 1 mg/l BAP, 8 g/l phytoagar, 200 μM Acetosyringone, |
| | 50 mg/l Thymidine |
| 17202I | 5 ml/l MS Vitamin, 30 g/l Sucrose, 2 mg/l 2,4-D, 0.25 mg/l BAP, 8 g/l phytoagar, |
| | 200 mg/l Timentin, 100 mg/l spectinomycin |
| 17203H | 30 g/l sucrose, 7 g/l phytoagar, 25 mg/l kanamycin |

### Example 1: Generation of Somatic Embryos in Cotton Explants and Regeneration of Transgenic Cotton Plants

Seeds of various cotton (Gossypium hirsutum) genotypes were surface-sterilized as described above. Following sterilization, seeds were washed in sterile water and then germinated and transferred to liquid elongation medium for 10-14 days at 28° C.; and expanded cotyledon explants were dissected into 1×1 mm to 5×5 mm pieces. In an alternative procedure, following sterilization and washing, seeds (including some Pima variety cotton seeds) were then germinated on semi-solid media 14580C for 1-5 days at 28° C. in dark and the emerging cotyledons were dissected into 1×1 mm or 2×2 mm pieces.

Agrobacterium strain LBA4404 THY-TD harboring a T-DNA expression construct was used for transformation. The expression construct included coding sequence(s) for (a) a single morphogenic gene (i.e., LEC2, BBM, or RKD4); (b) two morphogenic genes: BBM1 and LEC2 (SEQ ID NO:1); (c) two morphogenic genes: RKD4 and LEC2 (SEQ ID NO:2); or (d) no morphogenic genes (control). Each of the foregoing expression constructs with morphogenic genes, also included a heat-inducible CRE recombinase coding sequence and loxp excision sites flanking the BBM1/LEC2 or RKD4/LEC2 expression cassettes; and all three expression constructs included spectinomycin resistance conferred by a SPCN sequence and a visual DS-RED marker. Actively growing Agrobacterium were suspended in 14580AJ liquid medium adjusted to an OD of 0.3 at 600 nm. Explants were incubated with the Agrobacterium suspension in 100×25 mm petri dishes for 1 hour. Suspension medium was subsequently removed, and explants co-cultivated for T-DNA transfer. Explants were co-cultivated on wet filter paper with the foregoing 14580AJ (Thymidine+AS) medium in a growth chamber under low light (12 μmol m⁻² s⁻¹) for 3 days. In some cases, explants were then transferred to 14580K selection medium containing spectinomycin (spectinomycin resistance was conferred by SPCN sequence on each expression construct). In other cases, explants of varieties including Pima cotton varieties were transferred to (ii) 14580AM selection medium containing spectinomycin or (iii) 14580P medium (for constructs do not contain SPCN).

After induction of somatic embryos, explants were subjected to heat treatment at 45° C. for 2 hours once a week for 4 weeks to induce CRE expression and excise the loxp-flanked morphogenic genes. Heat-shocked somatic embryos were transferred to desiccation medium 17862 for 7 days, and then moved to germination medium 199M. Germinated somatic embryos were transferred to rooting and elongation medium 14582F for regeneration of transgenic plants.

PCR analysis of transgenic shoots verified the excision of morphogenic genes and the presence of a single copy of the SPCN transgene and DS-RED (which were located outside flanking sites in original construct).

The ability of BBM1 and LEC2 (“BBM1/LEC2”) or RKD4 and LEC2 (“RKD4/LEC2”) to generate somatic embryos was tested in 11 different genotypes: Coker 312 and ten normally recalcitrant elite cotton varieties. The results are shown Table 3.

TABLE 3

| | % Explants with Somatic Embryos | | | | | |
|---|---|---|---|---|---|---|
| | T-DNA | | | | | |
| | Control | | | | | |
| | (No Morph | | | | BBM1/ | RKD4/ |
| Genotype | Gene) | LEC2 | BBM1 | RKD4 | LEC2 | LEC2 |
| Coker 312 | 27% | 0% | 0% | 0% | 61% | — |
| GC510 | 0% | — | — | — | 12% | — |
| Elite line 1 | 0% | 0% | 0% | 0% | 45% | 10% |
| Elite line 2 | 0% | 0% | 0% | 0% | 50% | 20% |
| Elite line 3 | 0% | 0% | 0% | 0% | 55% | 60% |
| Elite line 4 | 0% | 0% | 0% | 0% | 32% | 20% |
| Elite line 5 | 0% | 0% | 0% | 0% | 20% | 25% |
| Elite line 6 | 0% | 0% | 0% | 0% | 60% | 70% |
| Elite line 7 | 0% | 0% | 0% | 0% | 80% | 45% |
| Elite line 8 | 0% | 0% | 0% | 0% | 50% | 25% |
| Elite line 9 | 0% | 0% | 0% | 0% | 25% | 5% |

The foregoing results demonstrate the unexpected ability of BBM1/LEC2 or RKD4/LEC2 to generate somatic embryos in cotton genotypes that were previously proven recalcitrant to somatic embryo induction. The foregoing results also show that BBM1/LEC2 surprisingly increased somatic embryo formation in the non-recalcitrant variety Coker 312, which was improved by more than 100%.

The percentages of cotton explants that regenerated T0 plants in 9 different genotypes tested are shown in Table 4.

TABLE 4

% Cotton Explants with T0 Plants
T-DNA

| Genotype | BBM1/LEC2 | RKD4/LEC2 |
|---|---|---|
| Elite line 1 | 45% | 10% |
| Elite line 2 | 125% | 0% |
| Elite line 3 | 15% | 5% |
| Elite line 4 | 10% | 0% |
| Elite line 5 | 85% | 10% |
| Elite line 6 | 15% | 25% |
| Elite line 7 | 90% | 40% |
| Elite line 8 | 75% | 0% |
| Elite line 9 | 110% | 5% |

Additionally, cotyledon explants of Pima 800 cotton (Gossypium barbadense) genotype were transformed as described above. Several hundred T0 events were recovered from the somatic embryos formed after heat-shock-mediated excision of the morphogenic genes BBM1 and LEC2. 180 T0 plants transformed with Agrobacterium strain LBA4404 THY-TD harboring the BBM1/LEC2 expression cassettes were randomly selected and qPCR analysis was carried out. 178 out of 180 T0 events were qPCR positive for the transgenes and 60 T0's showed successful removal of the Cre and BBM1/LEC2 expression cassettes, and contained only the gene of interest (GOI) cassette after heat shock treatment. The use of heat shock-inducible treatments resulted in a 33% excision frequency. Single-copy events for the GOI cassette were produced at a frequency of 25% in the Pima 800 genotype.

The foregoing results demonstrate what is believed to be the first reported successful regeneration of transgenic cotton plants from cotton varieties that are recalcitrant to somatic embryo induction and transformation without BBM1/LEC2 or RKD4/LEC2, including Pima cotton varieties. The foregoing also showed consistent and greatly improved transformation efficiency in explants that underwent BBM1/LEC2 supported embryogenesis.

Example 2: Fertility of Transgenic Events in Cotton Transformed Using BBM/LEC2. Transgenic shoots obtained from the transformation of different cotton genotypes in Example 1 were transferred to soil and grown in the greenhouse. After 4-5 months, bolls were produced and seeds collected. Almost all genotypes that produced events had fertility of 50% or greater. The number of events producing seeds and percentage of fertile events are shown in Table 5.

TABLE 5

| | | Events | % Fertile |
|---|---|---|---|
| Genotype | Events | producing seeds | Events |
| Elite line 1 | 43 | 26 | 60.5% |
| Elite line 3 | 2 | 1 | 50% |
| Elite line 4 | 1 | 0 | 0% |
| Elite line 5 | 8 | 6 | 75% |
| Elite line 6 | 2 | 1 | 50% |
| Elite line 7 | 4 | 2 | 50% |
| Elite line 8 | 4 | 2 | 50% |

The foregoing results demonstrate what is believed to be the first reported successful regeneration of fertile transgenic cotton plants from most of these cotton varieties. The foregoing also demonstrates the robust efficiency of the disclosed method to produce fertile transgenic cotton plants.

Example 3: Generation of Somatic Embryos in Alfalfa Explants. Clippings of green-house grown alfalfa (Medicago sativa) of various genotypes were surface-sterilized as described above. Following sterilization, trifoliate leaves were dissected into at least four pieces. Dissected alfalfa explants were incubated with Agrobacterium strain LBA4404 THY-TD harboring a T-DNA expression construct for BBM1 and LEC2 (SEQ ID NO:1), RKD4 and LEC2 (SEQ ID NO:2), or RKD4 and LEC1 (SEQ ID NO:3). Explants were incubated with the Agrobacterium suspension in petri dishes for 5 min. Explants were then removed and then co-cultivated in 17202B medium for 3 days. Explants were transferred to selection medium 172021 and the percentages of alfalfa explants that formed somatic embryos from explants of three different genotypes is shown in Table 6.

TABLE 6

| | % Alfalfa Explants with Somatic Embryos | | |
|---|---|---|---|
| | T-DNA | | |
| Genotype | RKD4/LEC1 | RKD4/LEC2 | BBM1/LEC2 |
| R97-037-005 | 100% | 100% | 100% |
| Alfalfa line 1 | 0% | 25% | 17% |
| Alfalfa line 2 | 0% | 67% | 50% |

The foregoing results demonstrate the unexpected ability of RKD4 and LEC2 (“RKD4/LEC2”) or BBM1 and LEC2 (“BBM1/LEC2”) to generate somatic embryos in alfalfa genotypes which were found to be otherwise recalcitrant to somatic embryo induction.

Example 4: Regeneration of Transgenic Alfalfa Plants from Somatic Embryos Somatic embryos from alfalfa (Medicago sativa) explants were generated as described in Example 3 by transformation with a T-DNA BBM1/LEC2 expression construct (SEQ ID NO: 1), a T-DNA with a RKD4/LEC2 expression construct (SEQ ID NO:2), or a RKD4/LEC1 expression construct (SEQ ID NO:3). In addition to the morphogenic genes, each expression construct also included a heat-inducible CRE recombinase coding sequence and loxp excision sites flanking the BBM1/LEC2, RKD4/LEC1, or RKD4/LEC2 expression cassettes. After somatic embryo induction, somatic embryos were subjected to heat treatment at 45° C. for 2 hours once a week for 4 weeks to induce CRE expression and excise the morphogenic gene expression cassettes. Heat-shocked somatic embryos were desiccated for 2 days, and then moved to germination medium 17203H for regeneration of transgenic plants. PCR analysis of transgenic shoots verified the excision of morphogenic genes and the presence of a single copy of the SPCN and DS-RED transgenes.

The percentages of alfalfa explants that regenerated T0 plants in 3 distinct genotypes are shown in Table 7.

TABLE 7

| | % Alfalfa Explants with T0 Plants | | |
|---|---|---|---|
| | T-DNA | | |
| Genotype | RKD4/LEC1 | RKD4/LEC2 | BBM1/LEC2 |
| R97-037-005 | 100% | 100% | 100% |
| Alfalfa line 1 | 0% | 25% | 33% |
| Alfalfa line 2 | 0% | 50% | 50% |

The foregoing results demonstrate the ability of RKD4 and LEC2 (“RKD4/LEC2”) or BBM1 and LEC2 (“BBM1/LEC2”) to regenerate transgenic plants from somatic embryos in alfalfa genotypes which were found to be otherwise recalcitrant to such transformation methods. For example, variety Alfalfa line 1 was previously found to be recalcitrant to transformation. Thus, this represents the first reported successful transformation of this recalcitrant variety.

Example 5: Generation of Somatic Embryos in Canola Explants. Seeds of canola (Brassica napus) genotype 4PYZE50B were surface-sterilized as described above. Following sterilization, seeds were washed in sterile water and germinated in 90 medium for 7-10 days. Hypocotyls were dissected into 3-4 mm pieces and expanded cotyledon explants were dissected into 5×5 mm pieces. Dissected explants were incubated with Agrobacterium strain LBA4404 THY-TD harboring a T-DNA expression construct for BBM1/LEC2 (SEQ ID NO:1), RKD4/LEC2 (SEQ ID NO:2), or without morphogenic genes (control). In addition to the morphogenic genes, each expression construct also included a heat-inducible CRE recombinase coding sequence and loxp excision sites flanking the BBM1/LEC2 or RKD4/LEC2 genes. Explants were infected with the Agrobacterium suspension in petri dishes for 10 min. The Agrobacterium suspension was subsequently removed, explants were co-cultivated in 20A medium for 3 days in a growth chamber under low light (12 μmol m⁻² s⁻¹).

Explants were then transferred to somatic embryo induction medium 70AY. Percentage of canola cotyledon and hypocotyl explants that formed somatic embryos are shown in Table 8.

TABLE 8

| | | % Canola Explants with Somatic Embryos | | |
|---|---|---|---|---|
| | | T-DNA | | |
| | Explant | Control (No | | |
| Genotype | Source | Dev Gene) | BBM1/LEC2 | RKD4/LEC2 |
| G00010BC | Cotyledon | 0% | 5% | 1.7% |
| | Hypocotyl | 0% | 51% | not determined |

The foregoing demonstrates the unexpected ability of RKD4 and LEC2 (“RKD4/LEC2”) or BBM1 and LEC2 (“BBM1/LEC2”) to generate somatic embryos in Brassica genotypes and tissue types, which were found to be recalcitrant to other methods of somatic embryo induction.

Example 6: Regeneration of Transgenic Brassica Plants from Somatic Embryos Somatic embryos from canola (Brassica napus) explants were generated as described in Example 5 transformed with the Agrobacterium strain LBA4404 THY-TD harboring a T-DNA expression constructs for BBM1/LEC2 (SEQ ID NO:1), RKD4/LEC2 (SEQ ID NO:2), or without morphogenic genes (control). After somatic embryo induction, somatic embryos were subjected to heat treatment at 45° C. for 2 hours once a day for 2 days to induce CRE expression and excise morphogenic genes. Heat-shocked somatic embryos were transferred to 90A medium for regeneration of transgenic plants. The number somatic embryos and canola explants that regenerated transgenic T0 plants were counted 40-56 days after infection. Percentages of cotyledon- or hypocotyl-derived somatic embryos that regenerated plants are shown in Table 9.

TABLE 9

| | | % Canola Explants that Regenerated T0 Plants | | |
|---|---|---|---|---|
| | | T-DNA | | |
| | Explant | Control | | |
| Genotype | Source | (No Dev Gene) | BBM1/LEC2 | RKD4/LEC2 |
| G00010BC | Cotyledon | 0% | 0% | 0% |
| | Hypocotyl | 0% | 29% | not determined |

The foregoing demonstrates the unexpected ability of BBM1 and LEC2 (“BBM1/LEC2”) to regenerate transgenic plants from hypocotyl-derived somatic embryos in Brassica lines that have were otherwise recalcitrant to such transformation methods.

Example 7: Generation of Somatic Embryos in Hypocotyls from Various Brassica Genotypes. Brassica napus somatic embryos were generated from hypocotyls of various genotypes as described in Example 5. Explants were transformed with Agrobacterium strain LBA4404 THY-TD harboring a BBM1/LEC2 expression construct, heat-inducible CRE gene, DS-RED gene, and SPCN selectable marker gene (SEQ ID NO:1). Somatic embryos were counted 41 days after infection, and the percentage of Brassica explants exhibiting somatic embryos in 3 different genotypes, including Brassica napus elite breeding lines are shown in Table 10.

TABLE 10

| | % Canola Explants |
|---|---|
| | with Somatic |
| Genotype | Embryos |
| Westar | 29% |
| NS1822BC | 35% |
| G00555MC | 44% |

The foregoing demonstrates the ability of BBM1 and LEC2 (“BBM1/LEC2”) to efficiently generate somatic embryos in hypocotyl tissue from elite Brassica lines.

Example 8: Generation of Somatic Embryos in California Poppy Explants and Transgenic Plants thereof. Seeds of California poppy (Eschscholzia californica) were surface-sterilized as described above. Following sterilization, seeds were washed in sterile water and germinated in medium 90 for 7-10 days.

Hypocotyls were dissected into 3-4 mm pieces and expanded cotyledon explants were dissected into 5×5 mm pieces. Dissected explants were incubated with the Agrobacterium strain LBA4404 THY-TD harboring a T-DNA with expression construct that included BBM1/LEC2, heat-inducible CRE gene, DS-RED gene, and SPCN selectable marker gene (SEQ ID NO:1). Explants were incubated with the Agrobacterium suspension in petri dishes for 10 minutes. The Agrobacterium suspension was subsequently removed, and explants were co-cultivated in 20A medium for 2-4 days in a growth chamber under low light (12 μmol m⁻² s⁻¹). Infected explants were transferred to 70AY medium for induction of somatic embryos.

After somatic embryo induction, somatic embryos were subjected to heat treatment at 45° C. for 2 hours once a week for 4 weeks to induce CRE expression and excise the morphogenic gene expression cassettes. Heat-shocked somatic embryos were transferred to regeneration medium and subcultured every 3 weeks until rooted transgenic plants were obtained.

Somatic embryos were counted 31 days after Agrobacterium infection and the percentage of poppy explants exhibiting somatic embryos are shown in Table 11. The percentage of explants from which T0 transgenic plants were recovered are also shown in Table 11.

TABLE 11

| | % Poppy Explants with | |
|---|---|---|
| Explant | Somatic Embryos | % T0 plants |
| Cotyledon | 28.2% | 5.5% |
| Hypocotyl | 22.8% | 11.8% |

The foregoing demonstrates the unexpected efficiency with which BBM1 and LEC2 (“BBM1/LEC2”) generate somatic embryos in poppy plant explants; as well as the ability of the foregoing protocol to generate transgenic poppy plants from the somatic embryos.

Example 9: Generation of Somatic Embryos in Tomato Explants and Transgenic Plants thereof. Seeds of tomato (Solanum lycopersicum) cv. Roma were surface sterilized as described above. Following sterilization, seeds were washed in sterile water and germinated in half-strength MS medium with 90 Base. Seedlings with a 6-10 cm long hypocotyl and expanded cotyledons were segmented. Hypocotyls were further dissected into 3-4 mm pieces and expanded cotyledon explants were dissected into 5×5 mm pieces. Dissected explants were incubated with the Agrobacterium strain LBA4404 THY-TD harboring a T-DNA expression constructs for BBM1/LEC2, a heat-inducible CRE gene, a DS-RED gene, and a SPCN selectable marker gene (SEQ ID NO:1). Explants were infected with Agrobacterium suspension in petri dishes for 10 minutes. The Agrobacterium suspension was subsequently removed, and the explants co-cultivated in 20A medium for 2-4 days in a growth chamber under low light (12 μmol m⁻² s⁻¹). Explants were then transferred to 70AY medium for somatic embryo induction.

After somatic embryo induction, somatic embryos were subjected to heat treatment at 45° C. for 2 hours once a week for 4 weeks to induce CRE expression and excise the morphogenic gene expression cassettes. Heat-shocked somatic embryos were transferred to regeneration medium and subcultured every 3 weeks until rooted transgenic plants were obtained.

The number explants generating somatic embryos were counted 49 days after Agrobacterium infection, and the percentages of tomato explants exhibiting somatic embryos are shown in Table 12. The percentage of explants from which T0 transgenic plants were recovered are also shown in Table 12.

TABLE 12

| | % Tomato Explants with | |
|---|---|---|
| Explant | Somatic Embryos | % T0 plants |
| Cotyledon | 14.3% | not determined |
| Hypocotyl | 19.0% | 1.5% |

The foregoing demonstrates the ability of BBM1 and LEC2 (“BBM1/LEC2”) to generate somatic embryos in tomato plants and to regenerate transgenic tomato plants from hypocotyl-derived somatic embryos.

Example 10: Generation of Somatic Embryos in Cucumber Explants and Transgenic Plants thereof. Seeds and leaves of cucumber (Cucumis sativus) cv. Summer Dance were surface-sterilized as described above. Following sterilization, seeds were washed in sterile water and germinated in medium 90. Seedlings with a 6-10 cm long hypocotyl and expanded cotyledons were segmented. Hypocotyls were further dissected into 3-4 mm pieces and both expanded cotyledon explants and leaves were each dissected into 5×5 mm pieces. Dissected explants were incubated with the Agrobacterium strain LBA4404 THY-TD harboring a T-DNA with a BBM1/LEC2 expression construct, a heat-inducible CRE recombinase coding sequence, and loxp excision sites flanking the BBM1/LEC2 genes (SEQ ID NO: 1). Explants were incubated with the Agrobacterium suspension in petri dishes for 10 minutes, the Agrobacterium suspension was then removed, and explants co-cultivated in 20A medium for 2-4 days in a growth chamber under low light (12 μmol m⁻² s⁻¹). Explants were then transferred to 70AY medium for somatic embryo induction.

After somatic embryo induction, somatic embryos were subjected to heat treatment at 45° C. for 2 hours once a week for 4 weeks to induce CRE expression and excise the morphogenic gene expression cassettes. Heat-shocked somatic embryos were transferred to regeneration medium and subcultured every 3 weeks until rooted transgenic plants were obtained.

Somatic embryos were counted 35 days after infection and percentage of cucumber explants exhibiting somatic embryos are shown in Table 13. The percentage of explants from which T0 transgenic plants were recovered are also shown in Table 13.

TABLE 13

| | % Cucumber Explants with | |
|---|---|---|
| Explant | Somatic Embryos | % T0 Plants |
| Cotyledon | 66.5% | 5.0% |
| Hypocotyl | 63.0% | 8.3% |
| Leaf | 100% | 30.4% |

The foregoing demonstrates the ability of BBM1 and LEC2 (“BBM1/LEC2”) to generate somatic embryos from cotyledon-, hypocotyl-, and leaf-derived cucumber tissue at very high efficiencies. The foregoing results also demonstrate the disclosed method's ability to efficiently regenerate transgenic cucumber plants from these somatic embryos.

Example 11: Generation of Somatic Embryos in Sunflower. Seeds of sunflower (Helianthus annuus) are surface-sterilized and germinated on medium 90. Seedlings with a 6-10 cm long hypocotyl, expanded cotyledons and leaves are segmented into 3-4 mm pieces (hypocotyls) and 5×5 mm squares (cotyledons and leaves). Dissected explants are incubated with the Agrobacterium strain LBA4404 THY-TD harboring a T-DNA with a BBM1/LEC2 or RKD4/LEC2 expression cassettes, a heat-inducible CRE recombinase coding sequence, and loxp excision sites flanking the BBM1/LEC2 genes (SEQ ID NO:1) or RKD4/LEC2 genes (SEQ ID NO:2). Explants are incubated with the Agrobacterium suspension in petri dishes for 10 minutes, the Agrobacterium suspension is then removed, and explants co-cultivated in 20A medium for 2-4 days in a growth chamber under low light (12 μmol m⁻² s⁻¹). Explants are then transferred to 70AY medium for somatic embryo induction.

After somatic embryo induction, somatic embryos are subjected to heat treatment at 45° C. for 2 hours once a week for 4 weeks to induce CRE expression and excise the morphogenic gene expression cassettes. Heat-shocked somatic embryos are transferred to regeneration medium and subcultured every 3 weeks until rooted transgenic plants are obtained.

The foregoing provides a method for using BBM1/LEC2 or RKD4/LEC2 to generate somatic embryos from cotyledon-, hypocotyl- or leaf-derived sunflower tissue. The foregoing also provides regeneration of transgenic sunflower plants from these somatic embryos.

Example 12: Generation of Somatic Embryos in Carrots. Seeds of carrot (Daucus carota) are surface-sterilized and germinated on medium 90. Seedlings with a 6-10 cm long hypocotyl, expanded cotyledons and leaves are segmented into 3-4 mm pieces (hypocotyls) and 5×5 mm squares (cotyledons and leaves). Dissected explants are incubated with the Agrobacterium strain LBA4404 THY-TD harboring a T-DNA with a BBM1/LEC2 or RKD4/LEC2 expression cassettes, a heat-inducible CRE recombinase coding sequence, and loxp excision sites flanking the BBM1/LEC2 genes (SEQ ID NO:1) or the RKD4/LEC2 genes (SEQ ID NO:2). Explants are incubated with the Agrobacterium suspension in petri dishes for 10 minutes, the Agrobacterium suspension is then removed, and explants co-cultivated in 20A medium for 2-4 days in a growth chamber under low light (12 μmol m⁻² s⁻¹). Explants are then transferred to 70AY medium for somatic embryo induction.

After somatic embryo induction, somatic embryos are subjected to heat treatment at 45° C. for 2 hours once a week for 4 weeks to induce CRE expression and excise the morphogenic gene expression cassettes. Heat-shocked somatic embryos are transferred to regeneration medium and subcultured every 3 weeks until rooted transgenic plants are obtained.

The foregoing provides a method for using BBM1/LEC2 or RKD4/LEC2 to generate somatic embryos from cotyledon-, hypocotyl-, or leaf-derived carrot tissue. The foregoing also provides regeneration of transgenic carrot plants from these somatic embryos.

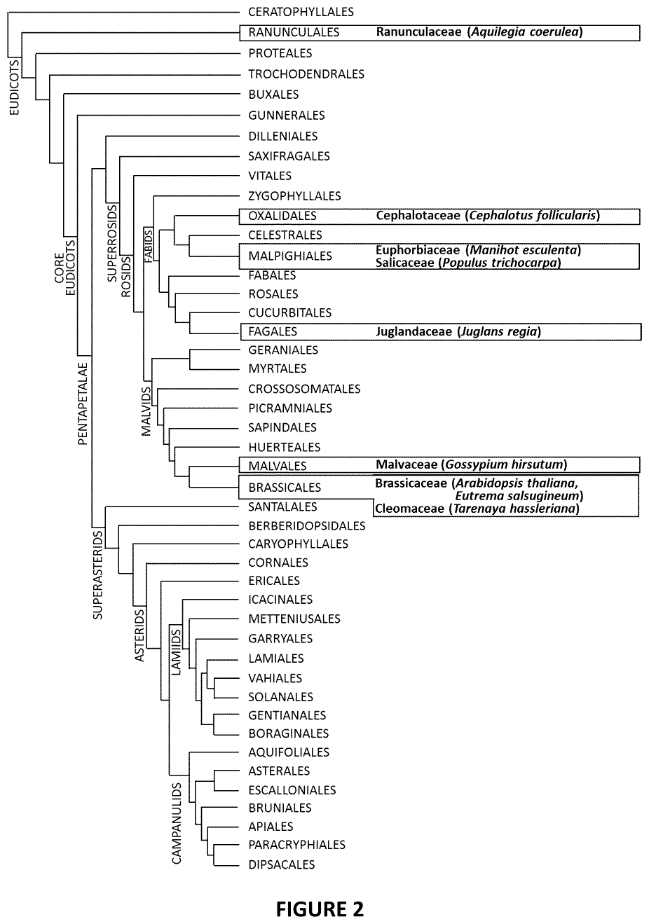

Example 13: Generation of Somatic Embryos in Cotton using Different LEC2 Gene Sources. Cotton cotyledon explants were prepared and transformed as described above in Example 1, with the following changes. Sterilized seeds were germinated on medium for 5 days and then dissected to expose cotyledons. Cotyledons were dissected into 1-2 mm pieces and infected with Agrobacterium suspension as described before. The Agrobacterium strain harbored a T-DNA with a BBM1/LEC2 expression cassette, a heat-inducible CRE recombinase coding sequence, and loxp excision sites flanking the BBM1/LEC2 genes (SEQ ID NO:1) except that the LEC2 gene sequence in the BBM1/LEC2 gene cassette in SEQ ID NO:1 was replaced with the coding sequence of a LEC2 gene from each of the sources indicated in Table 14. A phylogeny showing the relative positions of the different dicot species used as sources for LEC2 sequences is shown in FIG. 2. Number of explants that produced somatic embryos after 5 weeks were recorded and the frequency of somatic embryogenesis is given in Table 14.

TABLE 14

| | | | % Explants |
|---|---|---|---|
| Source of LEC2 in | | LEC2 | with Somatic |
| BBM1/LEC2 | LEC2 DNA | Protein | Embryos |
| Arabidopsis | SEQ ID NO: 6 | SEQ ID NO: 7 | 86% |
| thaliana | | | |
| Eutrema | SEQ ID NO: 12 | SEQ ID NO: 13 | 90% |
| salsugineum | | | |
| Gossypium hirsutum | SEQ ID NO: 14 | SEQ ID NO: 15 | 5% |
| Manihot esculenta | SEQ ID NO: 16 | SEQ ID NO: 17 | 100% |
| Cephalotus | SEQ ID NO: 18 | SEQ ID NO: 19 | 69% |
| follicularis | | | |
| Tarenaya | SEQ ID NO: 20 | SEQ ID NO: 21 | 100% |
| hasseleriana | | | |
| Juglans regia | SEQ ID NO: 22 | SEQ ID NO: 23 | 28% |
| Populus trichocarpa | SEQ ID NO: 24 | SEQ ID NO: 25 | 35% |
| Aquilegia coerulea | SEQ ID NO: 26 | SEQ ID NO: 27 | 13% |

The foregoing demonstrates that LEC2 sequences from multiple sources can be used in the methods disclosed herein that use BBM1/LEC2 to induce somatic embryos, including in their use of BBM1/LEC2 to generate somatic embryos with high (70%-90%) to very high (90%-100%) efficiency.

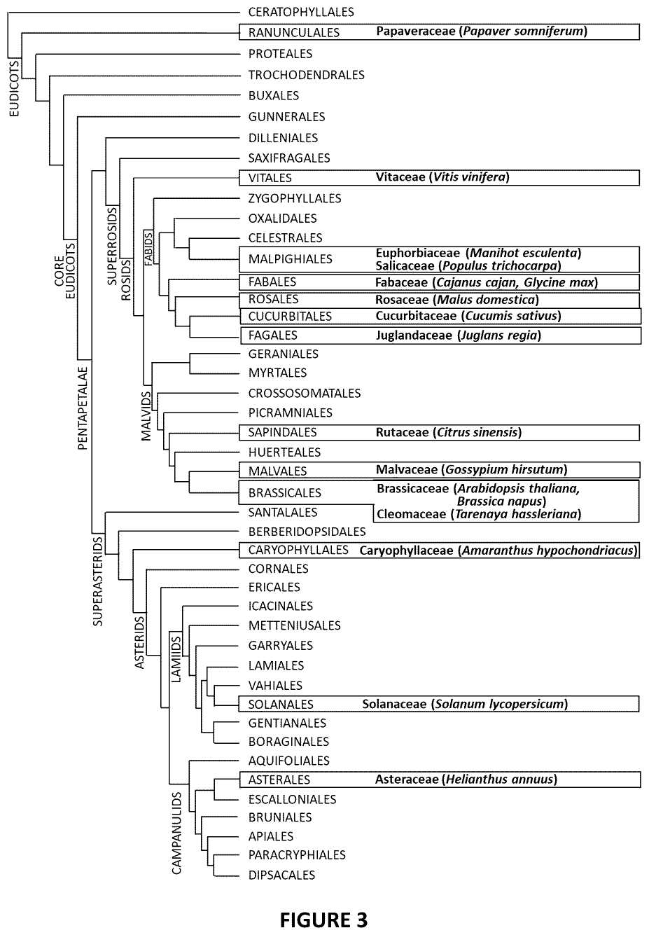

Example 14: Generation of Somatic Embryos in Cotton using Different BBM Gene Sources. Cotton cotyledon explants were prepared and transformed as described above in Example 13, except that the BBM1 gene in SEQ ID NO:1 was replaced with the coding sequence of a BBM gene from each of the sources indicated in Table 15. A phylogeny showing the relative positions of the different dicot species used as sources for BBM1 sequences is shown in FIG. 3. The Number of somatic embryos and T0 plants are assessed 4-5 weeks after infection.

TABLE 15

| | | | % Explants |
|---|---|---|---|
| Source of BBM1 in | | | with Somatic |
| BBM/LEC2 | BBM1 DNA | BBM1 Protein | Embryos |
| Brassica napus | SEQ ID NO: 4 | SEQ ID NO: 5 | 82.3% |
| Helianthus annuus | SEQ ID NO: 30 | SEQ ID NO: 31 | 100% |
| Amaranthus | SEQ ID NO: 32 | SEQ ID NO: 33 | not |
| hypochondriacus | | | determined |
| Cucumis sativa | SEQ ID NO: 34 | SEQ ID NO: 35 | 19.8% |
| Tarenaya | SEQ ID NO: 36 | SEQ ID NO: 37 | 97.9% |
| hassleriana | | | |
| Juglans regia | SEQ ID NO: 38 | SEQ ID NO: 39 | 25% |
| Populus trichocarpa | SEQ ID NO: 40 | SEQ ID NO: 41 | 13.5% |
| Cajanus cajan | SEQ ID NO: 42 | SEQ ID NO: 43 | 92.7% |
| Gossypium hirsutum | SEQ ID NO: 44 | SEQ ID NO: 45 | 89.6% |
| Manihot esculenta | SEQ ID NO: 46 | SEQ ID NO: 47 | 100% |
| Citrus sinensis | SEQ ID NO: 48 | SEQ ID NO: 49 | 92.7% |
| Vitis vinifera | SEQ ID NO: 50 | SEQ ID NO: 51 | not |
| | | | determined |
| Malus domestica | SEQ ID NO: 52 | SEQ ID NO: 53 | 90.9% |
| Solanum | SEQ ID NO: 54 | SEQ ID NO: 55 | 18.7% |
| lycopersicum | | | |
| Glycine max | SEQ ID NO: 75 | SEQ ID NO: 76 | 85.2% |
| Papaver somniferum | SEQ ID NO: 77 | SEQ ID NO: 78 | 37.2% |
| Zea mays | SEQ ID NO: 79 | SEQ ID NO: 80 | 27.1% |

The foregoing demonstrates the use of BBM1 sequences from multiple dicot and monocot (Zea mays) sources in the methods disclosed herein that use BBM1/LEC2 to induce somatic embryos with a range of efficiencies, including relatively low efficiency (0-19%), medium efficiency (20-69%), high efficiency (70-89%) and very high efficiency (90-100%).

Example 15: Generation of Somatic Embryos and T0 Transgenic plants in cotton using different promoters regulating BBM1 or LEC2. Cotton cotyledon explants were prepared and transformed as described above in Example 13, except that LEC2 was regulated by an Arabidopsis thaliana Ubiquitin 3 (UBQ3) promoter. Cotton explants were transformed with Agrobacterium strain LBA4404 THY-TD harboring a T-DNA comprising an expression construct that, in addition to heat-inducible CRE gene, DS-RED gene, and a SPCN gene, further included either a UBQ10::BBM-UBQ3::LEC2 expression cassette (SEQ ID NO: 65) or UBQ10::BBM-UBQ14::LEC2 expression cassette (SEQ ID NO:1). The frequency of explants producing somatic embryos, mature somatic embryos, mature somatic embryos transferred to rooting and T0 plants regenerated are given in Table 16. The quality of transgenic plants was assessed by PCR and T0s produced with UBQ3 promoter regulating LEC2 generally showed higher quality than those produced with UBQ14 promoter regulating LEC2. The frequency of high quality events with single copy of genes and complete excision of the morphogenic genes is given in Table 17.

TABLE 16

| | | | % of Mature | |
|---|---|---|---|---|
| | % Explants | % Explants with | Somatic | |
| | with Somatic | Mature Somatic | Embryos | % T0 |
| Promoter | Embryos | Embryos | to Rooting | Plants |
| UBQ14::LEC2 | 66.3% | 38.8% | 70.0% | 26.3% |
| UBQ3::LEC2 | 82.1% | 53.7% | 148.4% | 36.8% |

TABLE 17

| | | | Number of |
|---|---|---|---|
| | | Number of | excised T0s |
| | Number of | excised | with single copy |
| Promoter | T0s sampled | T0s (%) | of genes (%) |
| UBQ14::LEC2 | 54 | 11 (20%) | 2 (4%) |
| UBQ3::LEC2 | 57 | 38 (67%) | 24 (42%) |
| GM-EFTU2::LEC2 | 50 | 30 (60%) | 22 (44%) |

In additional examples, Glycine max Elongation Factor TU 2 (GM-EFTU2; SEQ ID NO: 66) or Rubisco Activase (GM-RUBACT; SEQ ID NO:67) promoter was used to regulate LEC2. When GM-EFTU2 promoter was regulating LEC2, 96.9% of explants produced somatic embryos, which is comparable to UBQ3 promoter (see Table 16). GM-RUBACT promoter did not produce any explants with somatic embryos. In other examples, one or more of BBM1, LEC2 and CRE are regulated by an auxin-inducible DR5 promoter (SEQ ID NO:72), an AT-UBI11 promoter (SEQ ID NO:81), or CSVMV promoter (SEQ ID NO:82).

The foregoing demonstrates that different promoters operably linked to BBM1, LEC2, and CRE can be used in the methods disclosed herein to induce somatic embryos and to very efficiently regenerate transgenic plants.

Example 16: Generation of Somatic Embryos in Cotton using CCAAT-Binding Factor 1A Activation Domain (CBF1A) coupled to BBM or LEC2. Cotton cotyledon explants were prepared and transformed as described above in Example 13, except that each morphogenic gene (BBM1 or LEC2) was coupled to CCAAT-Binding Factor 1A activation domain (CBF1A) to make BBM-CBF1A or LEC2-CBF1A. Cotton explants were transformed with Agrobacterium strain LBA4404 THY-TD harboring a T-DNA comprising an expression construct that, in addition to heat-inducible CRE gene, DS-RED gene, and a SPCN gene, further includes either a BBM-CBF1A/LEC2 expression cassette (SEQ ID NO:58); a BBM/LEC2-CBF1A (SEQ ID NO:59) expression cassette, or a BBM-CBF1A/LEC2-CBF1A (SEQ ID: 60) expression cassette. The number of explants that produced somatic embryos and then regenerate T0 transgenic plants was assessed. Results are shown in Table 18.

TABLE 18

| | % Explants with |
|---|---|
| Construct | Somatic Embryos |
| BBM1/LEC2 | 71.1% |
| BBM1 − CBF1A/LEC2 | 98.6% |
| BBM1/LEC2 − CBF1A | 91.2% |
| BBM1 − CBF1A/LEC2 − CBF1A | 28.4% |

The foregoing example shows the use CBF1A-coupled morphogenic genes in the disclosed methods for generating somatic embryos in dicot plants and/or regenerating transgenic plants from such somatic embryos.

### Example 17: Generation of Somatic Embryos in Cotton Using N-Terminus Truncated BBM with or without a Coupled CBF1A and Use of BBM AP2 Domain with or without a Coupled CBF1A

Cotton cotyledon explants are prepared and transformed as described above in Example 16, except that the expression construct's BBM1 gene sequence is replaced with a truncated version comprising the BBM AP2 domains and C-terminus but lacking part of the N-terminus such that the expression construct comprises either truncated BBM1 coupled to CBF1A or truncated BBM1 by itself. Somatic embryos are produced by either the truncated BBM1 by itself or the truncated BBM1 coupled to CBF1A. The number of explants that produce somatic embryos and then regenerate T0 transgenic plants is assessed.

In a separate experiment, cotton cotyledon explants were prepared and transformed as described above in Example 16, except that the expression construct's BBM1 gene sequence was replaced with a truncated version comprising the BBM AP2 domain such that the expression construct comprised either BBM AP2 domain-coupled to CBF1A (SEQ ID: 61 comprising BBM1 AP2-CBF1A/LEC2 sequences) or BBM1 AP2 by itself (SEQ ID: 61 comprising BBM1 AP/LEC2 genes). No somatic embryos were produced by either the BBM1 AP2 by itself or the BBM1 AP2 domain coupled to CBF1A.

The foregoing example shows the use CBF1A-coupled truncated morphogenic genes in the disclosed methods for generating somatic embryos in dicot plants and/or regenerating transgenic plants from such somatic embryos.

Example 18: Generation of Somatic Embryos in Cotton using Glucocorticoid Receptor (GR) coupled to BBM and/or LEC2. Cotton cotyledon explants were prepared and transformed as described above in Example 13, except that each morphogenic gene (BBM1 or LEC2) was coupled to Glucocorticoid Receptor (GR; SEQ ID NO:73) to make BBM-GR or LEC2-GR. Cotton explants were transformed with Agrobacterium strain LBA4404 THY-TD harboring a T-DNA comprising an expression construct that, in addition to heat-inducible CRE gene, DS-RED gene, and a SPCN gene, further included either a BBM-GR/LEC2 expression cassette; a BBM/LEC2-GR expression cassette, or a BBM-GR/LEC2-GR expression cassette. The morphogenic genes were induced by the addition of 0.5 mg/L triamcinolone acetonide (TA) to the medium. Most explants produced somatic embryos upon induction with TA for all the constructs described above. In the absence of TA, no somatic embryos were produced for the GR-linked morphogenic genes. For explants transformed with BBM1/LEC2, somatic embryos were produced regardless of absence or presence of TA. Somatic embryos were visualized by brightfield microscopy and also by DS RED fluorescence as shown in Table 19 (+ and − indicate presence or absence, respectively, of red fluorescent somatic embryos).

TABLE 19

Fluorescent Somatic Embryos

| Construct | Not Induced | TA-Induced |
|---|---|---|
| BBM1/LEC2 | + | + |
| BBM − GR/LEC2 | − | + |
| BBM/LEC2 − GR | − | + |
| BBM − GR/LEC2 − GR | − | + |

The foregoing example shows the use GR-coupled morphogenic genes in the disclosed methods for generating somatic embryos in dicot plants and/or regenerating transgenic plants from such somatic embryos.

Example 19: Retransformation of Transgenic Cotton Germplasm Containing dexamethasone-inducible BBM-GR and LEC2-GR to produce transgenic events with trait genes. Seeds from T0 plants produced in Example 18 are transformed with Agrobacterium strain LBA4404 THY-TD harboring a T-DNA comprising an expression construct with trait genes, as described in Example 13. Explants are placed on media supplemented with 5 mg/l of dexamethasone to induce somatic embryos and transgenic T0 plants with trait genes. Morphogenic gene cassettes are eliminated in subsequent generations by segregation, leaving transformed plants with trait genes.

The foregoing example provides a method for using transgenic germplasm for retransformation by generating somatic embryos in dicot plants and/or regenerating transgenic plants from such somatic embryos.

Example 20: Generation of Somatic Embryos in Cotton Using Auxin-Inducible CRE to excise BBM and LEC2 genes. Cotton cotyledon explants are prepared and transformed as described above in Example 13, except that CRE is regulated by an auxin-inducible promoter DR5 (SEQ ID NO:72). The Agrobacterium strain harbors a T-DNA with a BBM1/LEC2 expression cassette, an auxin-inducible DR5-CRE recombinase coding sequence, and loxp excision sites flanking the BBM1/LEC2 genes. After somatic embryos are induced, auxin is added to the medium to induce the expression of CRE for excising the BBM1 and LEC2 gene cassettes. The number of explants that produce somatic embryos and then regenerate T0 transgenic plants is assessed.

The foregoing example provides a method for using an auxin-inducible DR5 promoter regulating CRE expression for excision of morphogenic genes in the disclosed methods for generating somatic embryos in dicot plants and/or regenerating transgenic plants from such somatic embryos.

Example 21: Generation of Somatic Embryos in Cotton Using FUSCA3 (FUS3) Cotton cotyledon explants were prepared and transformed as described above in Example 13, except that the expression construct's LEC2 gene sequence was replaced with Arabidopsis thaliana FUSCA3 gene (DNA sequence SEQ ID NO:56 encoding protein of SEQ ID NO:57), Arabidopsis thaliana ABI3 gene (SEQ ID NO:63; encoding protein of SEQ ID NO:64), or Arabidopsis thaliana LEC1 gene (SEQ ID NO:10; encoding protein of SEQ ID NO:11). The number of explants that produced somatic embryos and then regenerate T0 transgenic plants was assessed. Results are shown in Table 20.

TABLE 20

| | % Explants with |
|---|---|
| Construct | Somatic Embryos |
| BBM1 + AT-FUS3 | 7.4% |
| BBM1 + AT-ABI3 | 0% |
| BBM1 + AT-LEC1 | 0% |

The foregoing example provides a method for using a FUSCA3 morphogenic gene in the disclosed methods for generating somatic embryos in dicot plants, at a low frequency relative to LEC2. No explants formed somatic embryos when LEC2 was replaced with AT-ABI3 or AT-LEC1.

Example 22: Generation of Transgenic Plants in Cotton using Glucocorticoid Receptor (GR) coupled to CRE for excision of Morphogenic Genes. Cotton cotyledon explants are prepared and transformed as described above in Example 13, except that the expression construct's CRE gene sequence is coupled to Glucocorticoid Receptor (GR; SEQ ID NO: 73) to make BBM1+LEC2+Constitutive Promoter::CRE-GR construct where BBM/LEC2 are flanked by loxP sites. Transformed cotton explants with somatic embryos forming on them are transferred to media containing TA (0.5 mg/L) to induce CRE-GR and facilitate excision of BBM1 and LEC2. The number of T0 plants regenerated is assessed.

The foregoing example provides a method for using a glucocorticoid-inducible constitutive promoter regulating CRE expression for excision of morphogenic genes in the disclosed methods for generating somatic embryos in dicot plants and/or regenerating transgenic plants from such somatic embryos.

Example 23: Automated Explant Preparation. Cotyledons from cotton seeds germinated from 3-15 days are dissected and transferred into Agrobacterium suspension in the bowl of a Cuisinart Mini-Blender (Model No. DLC-1SS) food processor. In other examples, hypocotyl tissue from canola or tomato or California poppy or cucumber, or leaf tissue from alfalfa or cucumber, or cotyledon tissue from canola, California poppy, tomato or cucumber are used instead of cotton cotyledons as explants.

The Cuisinart Mini-Blender food processor is then pulsed until the average explant length is approximately 2-3 mm. Explants are then either removed and contacted with Agrobacterium suspension for 10 minutes for infection or the Agrobacterium suspension is added to the bowl for 10 minute infection. Agrobacterium suspension and explants are then poured through a 0.4 μm nylon mesh, collecting explants on top. The nylon mesh is then inverted and tapped repeatedly to dislodge the tissue onto a filter paper resting on solid co-cultivation medium for 3 days. Explants are then moved to suitable medium for induction of somatic embryos. Explants are expected to produce somatic embryos after 4-5 weeks and T0 plants.

In a variation of the foregoing method, after infection with Agrobacterium suspension, explants are moved from the Cuisinart Mini-Blender food processor and distributed evenly on filter papers which are then placed on co-cultivation medium. Subsequently, the filter papers supporting explants are picked up using forceps and moved to fresh medium at each sub-culture step. Bulk transfer on filter paper of explants results in rapid formation of somatic embryos.

In a further variation, different food processors as well as automated or semi-automated grinding devices are used to prepare explants for transformation including the use of automated or semi-automated chopping and/or milling devices.

1. A method of producing dicot somatic embryo, the method comprising:
expressing in dicot explant a first heterologous nucleotide sequence encoding a BBM or a RWP-RK domain (RKD) polypeptide;
expressing in the dicot explant a second heterologous nucleotide sequence encoding a leafy cotyledon (LEC) polypeptide or FUSCA3 (FUS3) polypeptide; and
inducing somatic embryo formation in the explant.

2. The method of claim 1, wherein the first heterologous nucleotide sequence encodes BBM polypeptide and the second heterologous nucleotide sequence encodes LEC2 polypeptide.

3. The method of claim 1, wherein the first heterologous nucleotide sequence encodes RKD4 polypeptide and the second heterologous nucleotide sequence encodes LEC1 polypeptide.

4. The method of claim 1, wherein the first heterologous nucleotide sequence encodes RKD4 polypeptide and the second heterologous nucleotide sequence encodes LEC2 polypeptide.

5-8. (canceled)

9. The method of claim 1, wherein the dicot explant is from a recalcitrant dicot variety.

10. (canceled)

11. A method for producing a transgenic dicot plant, the method comprising:
transforming a dicot explant with a first heterologous nucleotide sequence encoding a BBM polypeptide or a RWP-RK domain (RKD) polypeptide and a second heterologous nucleotide sequence encoding a LEC polypeptide or a FUSCA3 (FUS3) polypeptide;
transforming the explant with a transgene;
inducing somatic embryo formation in the explant to produce a transgenic somatic embryo; and
culturing the transgenic somatic embryo tissue under germination, rooting, and shooting conditions to form a transgenic dicot plant comprising the transgene.

12. The method of claim 11, wherein the first heterologous nucleotide sequence encodes BBM polypeptide and the second heterologous nucleotide encodes LEC2 polypeptide.

13. The method of claim 11, wherein the first heterologous nucleotide sequence encodes RKD4 polypeptide and the second heterologous nucleotide sequence encodes LEC1 polypeptide.

14. The method of claim 11, wherein the first heterologous nucleotide sequence encodes RKD4 polypeptide and the second heterologous nucleotide sequence of encodes LEC2 polypeptide.

15-18. (canceled)

19. The method of claim 11, wherein the dicot explant is from a recalcitrant variety.

20-21. (canceled)

22. The method of any one of claim 11, wherein the dicot explant is an explant from cotton, alfalfa, Brassica napus, poppy, tomato, cucumber, sunflower, or carrot.

23. An expression construct comprising:
(a) a first promoter operably linked to a first heterologous nucleotide sequence encoding a BBM or a RWP-RK domain (RKD) polypeptide; and
(b) a second promoter operably linked to a second heterologous nucleotide sequence encoding a leafy cotyledon (LEC) polypeptide or a FUSCA3 (FUS3) polypeptide.

24. The expression construct of claim 23, wherein the first heterologous nucleotide sequence encodes BBM polypeptide and the second heterologous nucleotide sequence encodes LEC2 polypeptide.

25. The expression construct of claim 23, wherein the first heterologous nucleotide sequence encodes RKD4 polypeptide and the second heterologous nucleotide sequence of encodes LEC1 polypeptide.

26. The expression construct of claim 23, wherein the first heterologous nucleotide sequence encodes RKD4 polypeptide and the second heterologous nucleotide sequence encodes LEC2 polypeptide.

27-29. (canceled)

30. The expression construct of claim 23, wherein the first and second heterologous nucleotide sequences are flanked by recognition sites for a site-specific recombinase.

31. The expression construct of claim 30, further comprising a nucleotide sequence encoding the site-specific recombinase.

32. The expression construct of claim 31, wherein the nucleotide sequence encoding the site-specific recombinase is operably linked to a third promoter selected from an inducible promoter, a developmentally regulated promoter, or a constitutive promoter.

33. The method of claim 1, wherein the dicot explant is an explant from cotton, alfalfa, Brassica napus, poppy, tomato, cucumber, sunflower, or carrot.
